(19) United States
(12) Patent Application Publication
(10) Pub. No.: US 2026/0297633 A1
(43) Pub. Date: Oct. 1, 2026

(54) GENETICALLY MODIFIED YEAST WITH DECREASED ERYTHRITOL PRODUCTION

(71) Applicant: Cargill, Incorporated, Wayzata, MN (US)

(72) Inventors: Hans H. Liao, Plymouth, MN (US); Douglas Paul Lies, Otsego, MN (US); Catherine Bradshaw Poor, Minneapolis, MN (US)

(73) Assignee: Cargill, Incorporated, Wayzata, MN (US)

(21) Appl. No.: 19/480,850

(22) PCT Filed: May 2, 2024
(86) PCT No.: PCT/US2024/027357

§ 371 Date: Nov. 3, 2025

Related U.S. Application Data
(60) Provisional application No. 63/499,989, filed on May 4, 2023.

(51) Int. Cl. C12P 7/18; C12N 9/04

(52) U.S. Cl. CPC C12P 7/18; C12N 9/0006; C12Y 101/01021

(57) ABSTRACT

The present invention relates to methods for producing Saccharomyces strains that are capable of growth on xylose as a sole carbon source at a desired growth rate, (such as at least one generation per 48 hours), strains made by such methods, and Saccharomyces strains that grow at a growth rate of at least one generation per 48 hours using xylose as a sole carbon source for growth made by non-recombinant methods.

### CROSS-REFERENCE TO RELATED APPLICATIONS

This application claims the benefit of U.S. Provisional Application No. 63/499,989, filed May 4, 2023, which is incorporated by reference herein in its entirety.

### REFERENCE TO A SEQUENCE LISTING SUBMITTED VIA PATENT CENTER

The content of the Sequence Listing XML file of the sequence listing named “PT-1760-WO-PCT.xml” which is 177,337 bytes in size created on Apr. 29, 2024 and electronically submitted via Patent Center herewith the application is incorporated by reference in its entirety.

### BACKGROUND

Erythritol is a naturally occurring four-carbon sugar alcohol used as a food additive and sugar substitute. Erythritol is 60%-70% as sweet as sucrose but is noncaloric, does not affect blood sugar, and does not cause tooth decay. Common uses of erythritol include, beverages, such as coffee, tea, dietary supplements, juices, and soft drinks, and food products, such as confectionary, biscuits, cookies, table-top sweeteners, and chewing gum. Erythritol is produced commercially through the fermentation of substrates including dextrose using yeast. While yeast has been used in the production of erythritol by fermentation for many years, improvements to the fermentation process are beneficial in increasing yields and increasing production rate, leading to more sustainable and efficient fermentation processes. In contrast, the yeast that are used to produce erythritol may also be of use in the production of other polyols, so decreasing erythritol production in a yeast may also be beneficial. Accordingly, provided herein are genetically engineered yeast with modulated (i.e., increased or decreased) production of erythritol in fermentation processes using said engineered yeast.

### SUMMARY

The present disclosure provides a genetically engineered yeast cell, the engineered yeast cell comprising a deletion or disruption of a native gene encoding an erythrose reductase enzyme at least 60%, at least 65%, at least 70%, at least 75%, at least 80%, at least 85%, at least 90%, at least 95%, at least 98%, at least 99%, or 100% identical to at least one of SEQ ID NOs: 63 and 66. The yeast cell may be an osmotolerant yeast cell. The yeast cell may be a cell of the subphylum Ustilaginomycotina. The yeast cell may be selected form the group consisting of Trichosporonoides megachiliensis, Trychosporonoides oedocephalis, Trychosporonoides nigrescens, Pseudozyma tsukubaensis, Trigonopsis variabilis, Moniliella, Ustilaginomycetes, Trichosporon, Yarrowia lipolytica, Penicillium, Torula, Pichia, Candida, Candida magnoliae, and Aureobasidium.

The yeast cell may be a Moniliella pollinis cell and the gene encoding the erythrose reductase enzyme is at least 60%, at least 65%, at least 70%, at least 75%, at least 80%, at least 85%, at least 90%, at least 95%, at least 98%, at least 99%, or 100% identical to at least one of SEQ ID NOs: 61 and 64. The cell may have a deletion of at least one allele of the gene encoding the erythrose reductase enzyme. The cell may have a deletion of both alleles of the gene encoding the erythrose reductase enzyme.

When the engineered cell is used in a fermentation process in the presence of dextrose, titer and/or yield of erythritol production is decreased relative to titer and/or yield of erythritol in an equivalent fermentation process using an equivalent cell in which there has been no disruption or deletion of a native gene encoding an erythrose reductase at least 60%, at least 65%, at least 70%, at least 75%, at least 80%, at least 85%, at least 90%, at least 95%, at least 98%, at least 99%, or 100% identical to at least one of SEQ ID NOs: 63 and 66.

In the genetically engineered cells described herein, at least one copy of the gene encoding the erythrose reductase (ER) enzyme may be replaced with a native or exogenous polynucleotide sequence. The native or exogenous polynucleotide sequence may be operably linked to a heterologous or artificial promoter. The heterologous or artificial promoter may be selected from the group consisting of pyruvate kinase 1 promoter (PYK1p; SEQ ID NO:86), 6-phosphogluconate dehydrogenase promoter (6PGDp; SEQ ID NO:130), glyceraldehyde-3-phosphate dehydrogenase promoter (TDH3p; SEQ ID NO:132), translational elongation factor 1 promoter (TEFp; SEQ ID NO:133), modified TEFp (SEQ ID NO:131), phosphoglucomutase 1 promoter (PGMlp; SEQ ID NO:134), 3-phosphoglycerate kinase promoter (PGK1p; SEQ ID NO: 135), enolase promoter (ENO1p; SEQ ID NO:136), asparagine synthetase promoter (ASNSp; SEQ ID NO:137), 50S ribosomal protein L1 promoter (RPLAp; SEQ ID NO:138), and RPL16B (SEQ ID NO:139).

Also provided herein is a genetically engineered Moniliella pollinis cell comprising a deletion or disruption a native gene encoding an erythrose reductase enzyme at least 60%, at least 65%, at least 70%, at least 75%, at least 80%, at least 85%, at least 90%, at least 95%, at least 98%, at least 99%, or 100% identical to at least one of SEQ ID NOs: 63 and 66. The native gene may be at least 60%, at least 65%, at least 70%, at least 75%, at least 80%, at least 85%, at least 90%, at least 95%, at least 98%, at least 99%, or 100% identical to at least one of SEQ ID NOs: 61 and 64.

In the Moniliella pollinis cell, the native gene may be at least 60%, at least 65%, at least 70%, at least 75%, at least 80%, at least 85%, at least 90%, at least 95%, at least 98%, at least 99%, or 100% identical to SEQ ID NO:61 and the ER enzyme is at least 60%, at least 65%, at least 70%, at least 75%, at least 80%, at least 85%, at least 90%, at least 95%, at least 98%, at least 99%, or 100% identical to SEQ ID NO:63. The native gene may be at least 80% identical to SEQ ID NO:61 and the ER enzyme may be at least 80% identical to SEQ ID NOs: 63. The native gene may be at least 90% identical to SEQ ID NO:61 and the ER enzyme may be at least 85% identical to SEQ ID NO:63. The native gene is at least 95% identical SEQ ID NO: 61 and the ER enzyme may be at least 90% identical to SEQ ID NO:63. Both alleles of the native gene at least 60%, at least 65%, at least 70%, at least 75%, at least 80%, at least 85%, at least 90%, at least 95%, at least 98%, at least 99%, or 100% identical to SEQ ID NO:61 may be deleted.

In the Moniliella pollinis cell, the native gene may be at least 60%, at least 65%, at least 70%, at least 75%, at least 80%, at least 85%, at least 90%, at least 95%, at least 98%, at least 99%, or 100% identical to at least one of SEQ ID NO:64 and the ER enzyme is at least 60%, at least 65%, at least 70%, at least 75%, at least 80%, at least 85%, at least 90%, at least 95%, at least 98%, at least 99%, or 100% identical to at least one of SEQ ID NO:66. The native gene may be at least 80% identical to SEQ ID NO:64 and the ER enzyme may be at least 80% identical to SEQ ID NO:66. The native gene may be at least 90% identical to SEQ ID NO:64 and the ER enzyme may be at least 85% identical to SEQ ID NO:66. The native gene may be at least 95% identical to SEQ ID NO:64 and the ER enzyme may be at least 90% identical to SEQ ID NO:66. Both alleles of the native gene at least 60%, at least 65%, at least 70%, at least 75%, at least 80%, at least 85%, at least 90%, at least 95%, at least 98%, at least 99%, or 100% identical to SEQ ID NO:64 may be deleted.

The disclosure also provides a fermentation method comprising contacting a substrate comprising dextrose with an engineered cell described herein, wherein fermentation of the substrate by the engineered cell produces less erythritol than an equivalent call without the deletion or disruption. The fermentation temperature may be at or between 25° C. to 45° C., 30° C. to 40° C., or 32° C. to 37° C. The volumetric oxygen uptake rate (OUR) may be between 5-80, 10-75, 15-70, 20-60, 30-50, or 40-50 mmol O₂/(L·h). Rate, titer, and/or yield of erythritol production may be decreased relative to an equivalent fermentation run with an equivalent yeast cell in which the native gene encoding the erythrose reductase enzyme has not been deleted or disrupted. The concentration of dextrose may be at least 100 g/L.

The erythritol titer may be less than 20, 18, 15, 12, 10, 5, 1, 0.5 g/L when the fermentation is run at 35° C. for 96 hours. The erythritol production rate may be less than 0.5, 0.4, 0.3, 0.25, 0.2, 0.15, or 0.1 g L⁻¹ h⁻¹. The erythritol yield may be less than 35%, 30%, 25%, 20%, 15%, 10%, 5%, or 1%. The fermentation method may produce no erythritol or the process may be substantially free of erythritol.

Also provided herein is use of the genetically engineered cells described herein in a fermentation process.

### BRIEF DESCRIPTION OF THE FIGURES

This patent or application contains at least one drawing executed in color. Copies of this patent or patent application publication with color drawings will be provided by the Office upon request and the payment of the necessary fee.

The drawings illustrate generally, by way of example, but not by way of limitation, various aspects discussed herein.

FIG. 1 shows the native pentose phosphate pathway (dotted lines and arrows) and the native glycolysis pathways (solid lines and arrows) in Moniliella pollinis.

FIG. 2 shows enzyme activity for the in vitro assay described in Example 1.

FIG. 3 shows erythritol, xylitol, and glycerol metabolite concentrations (g/L) at 96 hours of shake flask fermentation of strains as outlined in Example 3. Data labels report the concentration of xylitol.

FIG. 4 shows erythritol, xylitol, and glycerol metabolite concentrations (g/L) at 96 hours of shake flask fermentation of strains as outlined in Example 4.

FIG. 5 shows erythritol, xylitol, and glycerol metabolite concentrations (g/L) at 96 hours of shake flask fermentation of strains as outlined in Example 5.

FIG. 6 shows erythritol, xylitol, and glycerol metabolite concentrations (g/L) at 96 hours of shake flask fermentations of strains as outlined in Example 6.

FIG. 7 shows erythritol, xylitol, and glycerol metabolite concentrations (g/L) at 96 hours of shake flask fermentations of strains as outlined in Example 7. Data labels report the concentration of erythritol.

FIG. 8 shows metabolite concentrations (g/L) for fermentations outlined in Example 8. Data labels report the concentration of erythritol.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis is also referred to as wt % or % (wt) below.

This disclosure relates to various recombinant cells engineered for decreased erythritol production. In general, the recombinant cells described herein are capable of producing erythritol and are characterized by deletion or disruption of a native gene encoding an erythrose reductase enzyme. The disclosure further provides fermentation methods using the engineered yeast described herein.

In general, recombinant cells described herein are yeast cells. As used herein, “yeast” refers to eukaryotic single celled microorganisms classified as members of the fungus kingdom. Yeast are unicellular organisms which evolved from multicellular ancestors with some species retaining multicellular characteristics such as forming strings of connected budding cells known as pseudo hyphae or false hyphae. Yeast cells may also be referred to in the art as yeast-like cells, and as used herein “yeast cell” encompasses both yeast and yeast-like cells. Suitable yeast and yeast-like host cells for modification may include, but are not limited to, Saccharomyces cerevisiae, Komagataella sp., Kluyveromyces (e.g., Kluyveromyces lactis, Kluveromyces marxianus), Yarrowia lipolytica, Issatchenkia orientalis, Pichia galeiformis, Pichia sp. YB-4149 (NRRL designation), Pichia pastoris, Candida (e.g., Candida magnoliae, Candida ethanolica), Pichia deserticola, Pichia membranifadens, Pichia fermentans, Aspergillus, Trichoderma, Myceliophthora thermophila, Moniliella (e.g., Moniliella pollinis), Pfaffia, Yamadazyma, Hansenula, Pichia kudriavzevii, Trichosporonoides (e.g., Trichosporonoides megachiliensis, Trychosporonoides oedocephalis, Trychosporonoides nigrescens), Pseudozyma tsukubaensis, Trigonopsis 1 variabilis, Penicillium, and Torula. An ordinarily skilled artisan would understand the requirements for selection of a suitable yeast cell, and recombinant yeast cells of the present disclosure are not limited to those expressly recited herein. Methods for genetic engineering of yeast cells are known and described in the art and a skilled artisan would understand the methods necessary to transform and engineer a suitable yeast cell.

A suitable yeast cell may be a cell of the phylum Basidiomycota and the subphylum Ustilaginomycotina. Suitable yeast of the subphylum Ustilaginomycotina include, but are not limited to, Ustilago (e.g., U. cynodontis, U. maydis, U. sphaerogena, U. cordal, U. scitaminea, U. coicis, U. syntherismae, U. esculenta, U. neglecta, U. crus-galli, Ustilago avenae), Sporisorium (e.g., Sporisorium exsertum), Moniliella (e.g., M. pollinis, M. tomentosa, M. acetoabutans, M. fonsecae, M. madida, M. megachiliensis, M. ocedocephalis, M. nigrescens), and Pseudozyma (e.g., Pseudozyma tsukubaensis), and Trichosporonoides (e.g., Trichosporonoides megachiliensis, Trychosporonoides oedocephalis, Trychosporonoides nigrescens). Yeast of the subphylum Ustilaginomycotina have been known and described in the art as potential production organisms for valuable chemicals such as itaconate, malate, succinate, mannitol, and erythritol and other valuable biotechnological applications. See, for example, Geiser et al. (Prospecting the biodiversity of the fungal family Ustilaginacceae for the production of value-added chemicals,” Fungal Biol Biotechnol, 2014, 1:2), Feldbrugge et al., (“The biotechnological use and potential of plant pathogenic smut fungi,” Appl Microbiol Biotechnol, 2013, 97(8):3253-65), Guevarra et al., (“Accumulation of itaconic, 2-hydroxyparaconic, itatartaric, and malic acids by strains of the genus Ustilago, Agric. Biol. Chem., 1990, 54(9), 2353-2358), and Moon et al., (“Biotechnological production of erythritol and its applications,” Appl Microbiol Biotechnol, 2010, 86:1017-1025).

A suitable yeast cell will have an active pentose phosphate pathway that produces ribulose-5-phosphate. As used herein “active pentose phosphate pathway” refers to expression of one or more functional enzymes which, together, convert glucose-6-phosphate, NADP⁺ or NAD+ (NAD(P)⁺), and water to NADPH or NADH (NAD(P)H), CO2, and ribulose-5-phosphate. Continuing in a non-oxidative phase, the pathway may also produce other pentose (i.e., 5-carbon) sugars. For example, the pentose phosphate pathway may produce ribulose-5-phosphate, ribose-5-phosphate, xylulose-5-phosphate, fructose 6-phosphate, combinations thereof, and the like, depending on the enzymatic activities present. The active pentose phosphate pathway may be native to the yeast cell or it may be introduced into the yeast cell by genetic engineering.

The yeast cell may be an osmotolerant yeast cell. As used herein, “osmotolerant” refers to a yeast capable of growth and reproduction under conditions of high osmolarity, such as at least 10% (w/v), at least 20% (w/v), at least 30% (w/v), at least 40% (w/v), at least 50% (w/v), or at least 60% (w/v) glucose and/or at least 6% (w/v), at least 10% (w/v), at least 12% (w/v), at least 13% (w/v), at least 15% (w/v) sodium chloride. Species and strains of osmotolerant yeast are known and described in the art, including many species of yeast used in industrial fermentation processes. Likewise, methods for assaying yeast osmotolerance are known and described in the art. See, for example, Tiwari, S., et al., (“Nectar yeast community of tropical flowering plants and assessment of their osmotolerance and xylitol-producing potential,” Current Microbiology, 2022, 79:28).

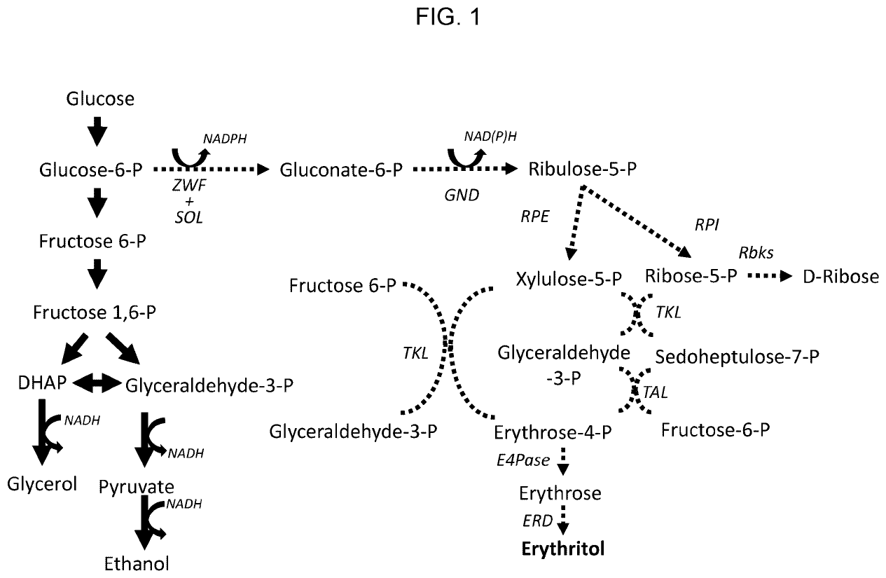

The recombinant yeast cell may be a recombinant Moniliella cell, for example, a Moniliella pollinis cell. FIG. 1 shows the predicted native pentose phosphate and glycolysis pathways in Moniliella pollinis. Moniliella has previously been used in the fermentation production of erythritol and methods for genetically modifying and fermenting Moniliella are known and described in the art. See, for example, Li et al. (“Methods for genetic transformation of filamentous fungi,” 2017, Microb Cell Fact, 16:168).

Various plasmids and methods for transformation of Moniliella are also described in the Examples below. For example, Moniliella may be transformed using a bipartite polynucleotide sequence in which, following recombination, the exogenous polynucleotide of interest is integrated at the specified locus and the selection marker is expressible within the cell. Suitable selection markers are known and used in the art. The selectable marker may include, but is not limited to, amdS (for example broken into a 3′ portion, SEQ ID NO:43, and a 5′ portion, SEQ ID NO:50), G418 resistance gene (for example broken into a 3′ portion, SEQ ID NO: 48, and a 5′ portion, SEQ ID NO:51), zeocin resistance gene (for example broken into a 3′ portion, SEQ ID NO:44, and a 5′ portion, SEQ ID NO:45), nourseothricin N-acetyl transferase (NAT) (for example broken into a 3′ portion, SEQ ID NO:47, and a 5′ portion, SEQ ID NO: 46), and invertase gene (SUC2) (for example a 3′ portion of SEQ ID NO:49 and a 5′ portion of SEQ ID NO:52).

The recombinant cells described herein may include one or more exogenous polynucleotide sequences encoding one or more polypeptides that, when expressed, modulate activity of the recombinant cell. For example, if the engineered cell is capable of producing a polyol (e.g., xylitol, ribitol, arabitol, etc.) integration of one or more exogenous polynucleotide sequences encoding one or more polypeptides may increase production of said polyol when the engineered yeast cell is used in a fermentation process to produce the polyol from glucose.

The terms “glucose” and “dextrose” are used interchangeably herein and refer to D-glucose except where expressly indicated otherwise.

As used herein, “exogenous” refers to genetic material or an expression product thereof that originates from outside of the host organism. For example, the exogenous genetic material or expression product thereof can be a modified form of genetic material native to the host organism, it can be derived from another organism, it can be a modified form of a component derived from another organism, or it can be a synthetically derived component. For example, a K. lactis invertase gene is exogenous when introduced into S. cerevisiae.

As used herein, “native” refers to genetic material or an expression product thereof that is found, apart from individual-to-individual mutations which do not affect function or expression, within the genome of wild-type cells of the host cell. For the purposes of this application, the Moniliella pollinis cell “Moniliella tomentosa var pollinis TCV364” described in U.S. Pat. No. 6,440,712, which is incorporated herein by reference in its entirety, and deposited under the Budapest Treaty at BCCM/MUCL (Belgian Coordinated Collections of Micro-organisms/Mycothèque de l'Université Catholique de Louvain by Eridania Béghin Say, Vilvoorde R&D Centre, Havenstraat 84, B-1800 Vilvoorde) on Mar. 28, 1997 under number MUCL40385, is considered the wild-type Moniliella pollinis cell.

As used herein, the terms “polypeptide” and “peptide” are used interchangeably and refer to the collective primary, secondary, tertiary, and quaternary amino acid sequences and structure necessary to give the recited macromolecule its function and properties. As used herein, “enzyme” or “biosynthetic pathway enzyme” refer to a protein that catalyzes a chemical reaction. The recitation of any particular enzyme, either independently or as part of a biosynthetic pathway is understood to include the co-factors, co-enzymes, and metals necessary for the enzyme to properly function. A summary of the amino acids and their three and one letter symbols as understood in the art is presented in Table 1. The amino acid name, three letter symbol, and one letter symbol are used interchangeably herein.

TABLE 1
Amino Acid three and one letter symbols

| | Three letter | One letter |
|---|---|---|
| Amino Acid | symbol | symbol |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Variants or sequences having substantial identity or homology with the polypeptides described herein can be utilized in the practice of the disclosed recombinant cells, compositions, and methods. Such sequences can be referred to as variants or modified sequences. That is, a polypeptide sequence can be modified yet still retain the ability to exhibit the desired activity. Generally, the variant or modified sequence may have greater than about 45%, 45%, 50%, 55%, 60%, 65%, 70%, 75%, 80%, 85%, 90%, or 95% sequence identity with the wild-type, naturally occurring polypeptide sequence, or with a variant polypeptide as described herein.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant,” “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge and/or hydrophobicity of the molecule at the site of the substitution, and/or (c) the bulk of the side chain.

As used herein, terms “polynucleotide,” “polynucleotide sequence,” and “nucleic acid sequence,” and “nucleic acid,” are used interchangeably and refer to a sequence of nucleotides or any fragment thereof. These phrases also refer to DNA or RNA of natural or synthetic origin, which may be single-stranded or double-stranded and may represent the sense or the antisense strand. The DNA polynucleotides may be a cDNA (e.g., coding DNA) or a genomic DNA sequence (e.g., including both introns and exons).

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

Those of skill in the art understand the degeneracy of the genetic code and that a variety of polynucleotides can encode the same polypeptide. In some aspects, the polynucleotides (e.g., polynucleotides encoding an erythrose reductase polypeptide) may be codon-optimized for expression in a particular cell including, without limitation, a plant cell, bacterial cell, fungal cell, or animal cell. While polypeptides encoded by polynucleotide sequences found in various species are disclosed herein any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

The recombinant cells described herein may include deletions or disruptions in one or more native genes. The phase “deletion or disruption” refers to the status of a native gene in the recombinant cell that has either a completely eliminated coding region (deletion) or a modification of the gene, its promoter, or its terminator (such as by a deletion, insertion, or mutation) so that the gene no longer produces an active expression product, produces severely reduced quantities of the expression product (e.g., at least a 75% reduction or at least a 90% reduction) or produces an expression product with severely reduced activity (e.g., at least 75% reduced or at least 90% reduced). The deletion or disruption can be achieved by genetic engineering methods, forced evolution, mutagenesis, RNA interference (RNAi), and/or selection and screening. Deletion or disruption of a native host cell gene can be coupled to the incorporation of one or more polynucleotide sequences (e.g., an exogenous or native polynucleotide sequence) into the host cell at the locus of the host cell gene to be deleted or disrupted. The polynucleotide sequence to be inserted may be designed to replace all or a portion of the host cell gene to be deleted or disrupted. The polynucleotide sequence may encode for a gene product of interest, for example, a polypeptide, an enzyme, and the like. The deletion or disruption can also be accomplished using a deletion construct that does not contain a polynucleotide sequence to be integrated. Other methods for gene disruption or deletion are known and described in the art.

The recombinant cells described herein may have a deletion or disruption in one or more native genes encoding an enzyme involved in erythritol fermentation or consumption. Deletion or disruption of one or more of these biosynthetic pathway enzymes decreases the ability of the recombinant cell to produce erythritol, thereby increasing carbon flux into the fermentation pathway for the production of other polyols (e.g., xylitol, arabitol, ribitol, etc.) or production of another fermentation product.

The recombinant cells described herein may include a deletion or disruption of a native erythrose reductase (ER) gene. The native ER gene encodes an enzyme that catalyzes the reversible conversion of erythrose or erythrose-4-phosphate to erythritol using a nicotinamide adenine dinucleotide (phosphate) (hydrogen) (NAD(P)(H)) cofactor. When the host cell contains multiple ER genes, it is preferred to delete or disrupt at least one of them. When the host cell contains multiple alleles of a given ER gene, it is preferred to delete or disrupt one allele or both alleles of the given ER gene.

As used herein, “NAD(P)H” refers to nicotinamide adenine dinucleotide (phosphate) hydrogen and is inclusive of both NADH and NADPH. As is understood in the art, inclusion of the phosphate (or P abbreviation) in parentheses indicates that the phosphate may be absent or precent and the name and abbreviation are inclusive of both. Similarly, “NAD(H)” or “NADP(H)” refers to both the reduced and oxidized forms of the cofactor.

When the recombinant cell is a Moniliella pollinis cell, the recombinant cell may comprise a deletion or disruption of an ER gene encoding an amino acid sequence at least 60%, at least 65%, at least 70%, at least 75%, at least 80%, at least 85%, at least 90%, at least 95%, at least 98%, at least 99%, or 100% identical to SEQ ID NO:66. When the recombinant cell is a Moniliella pollinis cell, the recombinant cell may comprise a deletion or disruption of an ER gene with a nucleotide sequence at least 60%, at least 65%, at least 70%, at least 75%, at least 80%, at least 85%, at least 90%, at least 95%, at least 98%, at least 99%, or 100% identical to SEQ ID NO:64.

When the recombinant cell is a Moniliella pollinis cell, the recombinant cell may comprise a deletion or disruption of an ER gene encoding an amino acid sequence at least 60%, at least 65%, at least 70%, at least 75%, at least 80%, at least 85%, at least 90%, at least 95%, at least 98%, at least 99%, or 100% identical to SEQ ID NO:63. When the recombinant cell is a Moniliella pollinis cell, the recombinant cell may comprise a deletion or disruption of an ER gene with a nucleotide sequence at least 60%, at least 65%, at least 70%, at least 75%, at least 80%, at least 85%, at least 90%, at least 95%, at least 98%, at least 99%, or 100% identical to SEQ ID NO:61.

The recombinant cells described herein may include one or more genetic modifications in which an exogenous nucleic acid is integrated into the genome of the host cell. One of skill in the art know how to select suitable loci in a yeast genome for integration of the exogenous nucleic acid. Suitable integration loci may include, but are not limited to, the PDC1, GPD1, CYB2A, CYB2B, g4240, YMR226, MDHB, ATO2, Adh9091, Adh1202, ADE2, ADH2556, GAL6, MDH1, SCW11, ER1, ER3, pyrF, TRP3, gpdIIA, and gpdIIB loci. For example, in a M. pollinis host cells, suitable interaction loci may include, but are not limited to, the ER1 locus (defined as the locus flanked by SEQ ID NO:3 and SEQ ID NO:38), the ER3 locus (defined as the locus flanked by SEQ ID NO:31 and SEQ ID NO:41), the PDC1 locus (defined as the locus flanked by SEQ ID NO:28 and SEQ ID NO:40), the pyrF locus (defined as the locus flanked by SEQ ID NO:29 and SEQ ID NO:39), the TRP3 locus (defined as the locus flanked by SEQ ID NO:32 and SEQ ID NO:35), the gpdIIA locus (defined as the locus flanked by SEQ ID NO:33 and SEQ ID NO:37); and the gpdIIB locus (defined as the locus flanked by SEQ ID NO:34 and SEQ ID NO:42). The exogenous nucleic acid may also be integrated in an intergenic region or other location in the host cell genome not specifically specified herein. Other suitable integration loci may be determined by one of skill in the art. Furthermore, one of skill in the art would recognize how to use sequences to design primers to verify correct gene integration at the chosen locus.

The recombinant cell may have one or more copies of a given exogenous nucleic acid sequence integrated in a host chromosome(s) and replicated together with the chromosome(s) into which it has been integrated. For example, the yeast cell may be transformed with nucleic acid construct including a polynucleotide sequence encoding for a polypeptide described herein and the polynucleotide sequence encoding for the polypeptide may be integrated in one or more copies in a host chromosome(s). The recombinant cell may include multiple copies (two or more) of a given polynucleotide sequence encoding a polypeptide described herein. The recombinant cell may have one, two, three, four, five, six, seven, eight, nine, ten, or more copies of a polynucleotide sequence encoding a polypeptide described herein integrated into the genome. The multiple copies of said polynucleotide sequence may all be incorporated at a single locus or may be incorporated at multiple loci.

Recombinant cells described herein may be characterized by overexpression of an ER enzyme and/or include an exogenous polynucleotide sequence encoding an ER enzyme. The ER enzyme may be native to the host cell or the ER enzyme may be an exogenous ER enzyme. For example, when the cell is Moniliella pollinis the ER enzyme may be an enzyme with a sequence at least 50%, at least 60%, at least 70%, at least 80%, at least 85%, at least 90%, at least 95%, at least 97%, or at least 99% identical to at least one of SEQ ID NOs: 63 and 66. The recombinant cell may comprise an exogenous polynucleotide sequence encoding an ER enzyme with a sequence at least 50%, at least 60%, at least 70%, at least 80%, at least 85%, at least 90%, at least 95%, at least 97%, or at least 99% identical to at least one of SEQ ID NOs: 63 and 66. The recombinant cell may include a genetic modification that increases expression of an ER enzyme at least 60%, at least 65%, at least 70%, at least 75%, at least 80%, at least 85%, at least 90%, at least 95%, at least 98%, at least 99%, or 100% identical to at least one of SEQ ID NOs: 63 and 66. The genetic modification may include, but is not limited to, insertion of additional copies of a nucleic acid encoding the native ER into the cell, insertion of a constitutive promoter upstream of the coding region of the native ER gene in the genome of the host cell, and/or modification of the existing promoter upstream of the coding region of the native ER gene in the genome of the host cell. One of skill in the art will recognize that expression of a native ER gene may be increased by a number of methods known in the art and will be able to select and apply such methods as appropriate. For example, additional copies of a native ER gene may be integrated into the genome of the host cell to increase the number of copies of the native ER gene in the engineered cell.

As used herein, “overexpression” refers to an expression level of a polypeptide that is higher than the expression level of the same polypeptide in the absence of a genetic modification or exogenous polynucleotide encoding said polypeptide in an equivalent cell.

In addition to producing erythritol, the engineered yeast cells described herein may also be capable of producing another polyol (e.g., xylitol, arabitol, ribitol, etc.) or for producing another fermentation product. Accordingly, it might be desirable to increase or decrease erythritol production in the engineered cell based on the other metabolites produced during the fermentation process and what yield of said metabolites is desired.

For example, the recombinant cell including a deletion or disruption of an ER gene may also be capable of producing xylitol and may comprise one or more exogenous polynucleotide sequences integrated into its genome. Examples of suitable recombinant yeast cells, polynucleotide sequences, and biosynthetic pathways for producing xylitol are described in at least U.S. Provisional Application No. 63/364,363, U.S. Provisional Application No. 63/364,370, U.S. Provisional Application No. 63/364,375, and U.S. Provisional Application No. 63/364,382, all filed May 9, 2022, each of which is incorporated by reference in its entirety.

In another example, the recombinant cell including a deletion or disruption of an ER gene may also be capable of producing arabitol and may comprise one or more exogenous polynucleotide sequences integrated into its genome. Examples of suitable recombinant yeast cells, polynucleotide sequences, and biosynthetic pathways for producing arabitol are described in at least U.S. Provisional Application No. 63/364,359 and U.S. Provisional Application No. 63/364,380, both filed May 9, 2022, each of which is incorporated herein by reference in its entirety.

The polynucleotides in the recombinant cells described herein may be under the control of a promoter. For example, the native or exogenous polynucleotide sequences may be operably linked to a heterologous or artificial promoter. Suitable promoters are known and described in the art. Promoters may include, but are not limited to, pyruvate decarboxylase promoter (PDC), translation elongation factor 2 promoter (TEF2), SED1, alcohol dehydrogenase 1A promoter (ADH1), hexokinase 2 promoter (HXK2), FLO5 promoter, pyruvate kinase 1 promoter (PYK1p; SEQ ID NO:4); 6-phosphogluconate dehydrogenase promoter (6PGDp; SEQ ID NO:6); glyceraldehyde-3-phosphate dehydrogenase promoter (TDH3p; SEQ ID NO:8); translational elongation factor 1 promoter (TEFp; SEQ ID NO:9); modified TEFp (SEQ ID NO:7); phosphoglucomutase 1 promoter (PGMlp; SEQ ID NO:10); 3-phosphoglycerate kinase promoter (PGK1p; SEQ ID NO:11); enolase promoter (ENO1p; SEQ ID NO:12); asparagine synthetase promoter (ASNSp; SEQ ID NO:13); 50S ribosomal protein L1 promoter (RPLAp; SEQ ID NO:14); and RPL16B (SEQ ID NO:15).

The polynucleotides in the recombinant cells described herein may be under the control of a terminator. For example, the exogenous or native polynucleotides may be operably linked to a heterologous or artificial terminator. Suitable terminators are known and described in the art. Terminators may include, but are not limited to, GAL10 terminator, PDC terminator, transaldolase terminator (TAL) 6PGD terminator (6PGDt; SEQ ID NO:16); ASNS terminator (ASNSt; SEQ ID NO:17); ENO1 terminator (ENO1t; SEQ ID NO:18); hexokinase 1 terminator (HXK1t; SEQ ID NO:19); PGK1 terminator (PGK1t; SEQ ID NO:20); PGM1 terminator (PGM1t; SEQ ID NO:21); PYK1 terminator (PYK1t; SEQ ID NO:22); RPLA terminator (RPLAt: SEQ ID NO:23); transaldolase 1 terminator (TAL1t; SEQ ID NO:24); TDH3 terminator (TDH3t; SEQ ID NO:25); translation elongation factor 2 terminator (TEF2t; SEQ ID NO: 26); triosephosphate isomerase 1 terminator (TPI1t; SEQ ID NO:27); and MpTEF1 (SEQ ID NO:60).

A promoter or terminator is “operably linked” to a given polynucleotide (e.g., a gene) if its position in the genome or expression cassette relative to said polynucleotide is such that the promoter or terminator, as the case may be, performs its transcriptional control function.

The polypeptides described herein may be provided as part of a construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. The construct may be a vector including a promoter operably linked to the polynucleotide encoding a polypeptide as described herein. As used herein, the term “vector” refers to a polynucleotide capable of transporting another polynucleotide to which it has been linked. The vector may be a plasmid, which refers to a circular double-stranded DNA loop into which additional DNA segments may be integrated.

The disclosure also provides fermentation methods using the recombinant cells described herein. The fermentation method may be used for producing another polyol (e.g., xylitol, arabitol, ribitol, etc.) or for producing another fermentation product. Accordingly, it might be desirable to increase or decrease erythritol production in the engineered cell based on the other metabolites produced during the fermentation process and what yield of said metabolites is desired.

The fermentation methods include the step of fermenting a substrate using the genetically engineered yeasts described herein. The fermentation method can include additional steps, as would be understood by a person skilled in the art. Non-limiting examples of additional process steps include maintaining the temperature of the fermentation broth within a predetermined range, adjusting the pH during fermentation, and isolating a fermentation product from the fermentation broth. The fermentation process may be a fully aerobic or a partially aerobic process.

The fermentation method can be run using a suitable fermentation substrate. The substrate of the fermentation method can include glucose, sucrose, galactose, mannose, molasses, xylose, fructose, hydrolysates of starch, lignocellulosic hydrolysates, or a combination thereof. One skilled in the art will recognize what fermentation substrate is suitable for a given fermentation organism and system.

The fermentation process can be run under various conditions. The fermentation temperature, i.e., the temperature of the fermentation broth during processing, may be ambient temperature. Alternatively, or additionally, the fermentation temperature may be maintained within a predetermined range. For example, the fermentation temperature can be maintained in the range of 25° C. to 45° C., 30° C. to 40° C., or 32° C. to 37° C., preferably about 35° C. However, a skilled artisan will recognize that the fermentation temperature is not limited to any specific range or temperature recited herein and may be modified as appropriate.

The fermentation process can be run within certain oxygen uptake rate (OUR) ranges. The volumetric OUR of the fermentation process can be in the range of 5-80, 10-75, 15-70, 20-60, 30-50, or 40-50 mmol O₂/(L·h). In some embodiments, the specific OUR can be in the range of 0.05 to 10, 0.1 to 9, 0.5 to 8, 1.0 to 7, 1.5 to 6, 2 to 5, or 2.5 to 4 mmol O₂/(g cell dry weight·h). However, the volumetric or specific OURs of the fermentation process are not limited to any specific rates or ranges recited herein.

The fermentation process can be run at various cell concentrations. In some embodiments, the cell dry weight at the end of fermentation can be 5 to 40, 8 to 30, or 10 to 20 g cell dry weight/L. Further, the pitch density or pitching rate of the fermentation process can vary. In some embodiments, the pitch density can be 0.05 to 11, 0.1 to 10, or 0.25 to 8 g cell dry weight/L.

The initial dextrose concentration of the fermentation may be at least 100, 200, 250, 300, 350, or at least 400 g/L dextrose when run as a batch fermentation. The initial dextrose concentration may be between 100 to 500, 150 to 450, 400 to 200, or 250 to 350 g/L when run as a batch fermentation. When run in a fed batch fermentation process, the dextrose concentration may be at least 100, 200, 250, 300, 350, 400, or at least 450 g/L.

The fermentation process can be associated with various characteristics, such as, but not limited to, fermentation production rate, pathway fermentation yield, final titer, and peak fermentation rate. These characteristics can be affected by the selection of the yeast and/or genetic modification of the yeast used in the fermentation process. These characteristics can be affected by adjusting the fermentation process conditions. These characteristics can be adjusted via a combination of yeast selection or modification and the selection of fermentation process conditions.

Using engineered cells in which an ER gene is overexpressed, the erythritol production rate of the process may be at least 0.2, 0.3, 0.5, 0.75, or at least 1.0 g L⁻¹ h⁻¹. The erythritol mass yield of the process may be at least 25, at least 30, at least 35, at least 40, at least 50, at least 55 percent, at least 65 percent, at least 70 percent, at least 75 percent, at least 80 percent, or at least 85 percent. The final erythritol titer of the process may be at least 5, 10, 20, 30, 50, 75, or 100 g/L.

Using engineered cells in which at least one copy of an ER gene had been deleted or disrupted, the erythritol production rate of the process may be less than 0.5, 0.4, 0.3, 0.25, 0.2, 0.15, or 0.1 g L⁻¹ h⁻¹. The erythritol mass yield of the process may be less than 35%, 30%, 25%, 20%, 15%, 10%, 5%, or 1%. The final erythritol titer of the process may be less than 20, 18, 15, 12, 10, 5, 1, 0.5 g/L.

The fermentation process can be run as a dextrose-fed batch. Further, the fermentation process can be a batch process, continuous process, or semi-continuous process, as would be understood by a person skilled in the art.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

Throughout the Examples, strain numbering and sequence identification numbers are used consistently. For example, strain 1-7 in Example 2 is the same as strain 1-7 in Examples 3, 4, 5, 7, etc.

### Example 1: Erythrose Reductase Candidate In Vitro Enzyme Assays

Polynucleotides encoding suspected erythrose reductase (ER) enzymes) (Table 2) were cloned into a vector containing a T7 promoter and terminator for cell-free protein expression using purified recombinant T7 RNA polymerase for transcription and purified recombinant ribosome for translation (New England Biolabs, PURExpress® In Vitro Protein Synthesis). Enzyme expression using the in vitro system was confirmed by gel electrophoresis (results not shown)

TABLE 2

| | | Gene Sequence | Gene Sequence | Amino Acid |
|---|---|---|---|---|
| | | (with introns) | (without introns) | Sequence |
| Source Organism | Gene Name | SEQ ID NO: | SEQ ID NO: | SEQ ID NO: |
| Moniliella pollinis | RCSR18717 | 61 | 62 | 63 |
| Moniliella pollinis | RCSR26640 | 64 | 65 | 66 |
| Moniliella pollinis | RCSR11551 | 67 | 68 | 69 |
| Moniliella pollinis | RCSR18367 (“ER1”) | 76 | 77 | 78 |
| Moniliella pollinis | RCSR08378 (“ER3”) | 79 | 80 | 81 |

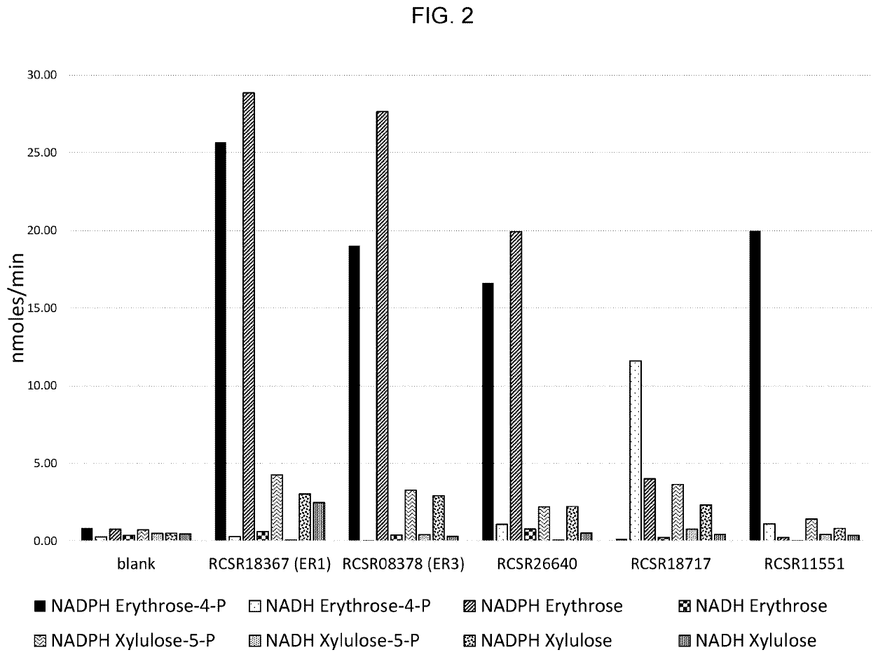

Cell-free synthesized proteins were analyzed for activity on four substrates (erythrose, erythrose-4-phosphate, xylulose, and xylulose-5-phosphase) with either NADPH or NADH cofactors. Enzyme assays were carried out in 96-well plates with 180 μL of reaction mixture as outlined in Table 3 and 20 μL of the in vitro enzyme expression product. Reaction progress over 15 minutes was monitored by the NAD(P)H concentration as measured by light absorbance at 340 nm. Results are shown in Table 4 and FIG. 2.

TABLE 3

| Component | final conc (mM) |
|---|---|
| HEPES, pH 8 | 50 |
| ZnSO4 | 0.001 |
| NADPH -OR- | 0.25 |
| NADH | |
| Erythrose-4-P -OR- | 3 |
| Erythrose -OR- | |
| Xylulose-5-P -OR- | |
| Xylulose | |
| water | to 180 uL |

TABLE 4

| | Enzyme Activity (nmoles/min) | | | | | | | |
|---|---|---|---|---|---|---|---|---|
| | NADPH cofactor | | | | NADH cofactor | | | |
| | erythrose-4- | | xylulose-5- | | erythrose-4- | | xylulose-5- | |
| ER Candidate | phosphate | erythrose | phosphate | xylulose | phosphate | erythrose | phosphate | xylulose |
| blank | 0.84 | 0.77 | 0.71 | 0.50 | 0.28 | 0.37 | 0.49 | 0.46 |
| RCSR18717 | 0.16 | 4.01 | 3.64 | 2.32 | 11.61 | 0.23 | 0.76 | 0.43 |
| RCSR26640 | 16.62 | 19.92 | 2.21 | 2.24 | 1.08 | 0.77 | 0.05 | 0.52 |
| RCSR11551 | 19.97 | 0.23 | 1.41 | 0.82 | 1.11 | 0.03 | 0.43 | 0.38 |
| RCSR18367 “ER1” | 25.68 | 28.86 | 4.26 | 3.04 | 0.30 | 0.61 | 0.07 | 2.47 |
| RCSR08378 “ER3” | 19.00 | 27.65 | 3.29 | 2.92 | 0.01 | 0.39 | 0.41 | 0.30 |

### Example 2—Genetically Modified Moniliella pollinis Strains

The strains described in this example were developed to test in vivo modulation (increase or decrease) of erythritol production through knockouts of the candidate ER genes, reduced expression of the candidate ER enzymes, and/or overexpression of candidate ER enzymes. A summary of the engineered strains is provided in Table 5.

### Strain 1-1

Strain 1-1 is the Moniliella pollinis host strain “Moniliella tomentosa var pollinis TCV364” described in U.S. Pat. No. 6,440,712, which is incorporated herein by reference in its entirety, and deposited under the Budapest Treaty at BCCM/MUCL (Belgian Coordinated Collections of Micro-organisms/Mycothèque de l'Université Catholique de Louvain by Eridania Béghin Say, Vilvoorde R&D Centre, Havenstraat 84, B-1800 Vilvoorde) on Mar. 28, 1997 under number MUCL40385.

### Strain 1-2

Strain 1-1 was transformed with SEQ ID NO:2 and SEQ ID NO:53 by first protoplasting the parent strain by adding an enzyme mixture containing 0.6M MgSO₄, 7.5 g/L driselase, and 12.5 g/L Trichoderma harzianum lysing enzyme to a mycelial pellet of the parent strain. Protoplasts were then pelleted, washed with 0.6M MgSO₄, and resuspended in STC medium (0.6M sucrose, 50 mM CaCl2, 10 mM Tris-HCl, pH 7.5). Bipartite transformations were prepared by adding 100 μg single stranded salmon sperm DNA and 1.5 to 5 μg each of the 5′ and 3′ DNA transformation fragments (3-10 μg total) to approximately 200 μL protoplast mixture (10⁸ cells/mL). 1 mL 50% PEG in STC medium was then added to the salmon sperm DNA, transformation DNA, and protoplast mixture and the resulting combination was incubated for 15 minutes at room temperature. Following incubation, recovery broth (0.4M sucrose, 1 g/L yeast extract, 1 g/L malt extract, 10 g/L glucose, pH 4.5) was added to the mixture and incubated at 27° C., 100 rpm, for 16 to 24 hours. Following the incubation, protoplasts were pelleted by centrifugation and resuspended in 1 mL PBS. The resuspended protoplasts were plated on PDA+250 mg/L geneticin (G418) selection plates and incubated at 35° C. for at least 2 days until transformants grew. Resulting transformants were streaked for single colony isolation on PDA+geneticin (G418) plates and single colonies were selected. Selected colonies were evaluated by colony PCR for integration of two copies of the L. rhamnosus XPDH sequence. A PCR verified isolate was designated strains 1-2.

SEQ ID NO:53 contains (i) a 3′ portion of the G418 selectable marker (SEQ ID NO: 48); ii) an expression cassette for the XPDH homolog from Lactobacillus rhamnosus, SEQ ID NO: 5 encoding the amino acid sequence of SEQ ID NO:1, under the control of the PYK1 promoter of SEQ ID NO:4 and the PGD terminator of SEQ ID NO:16; and (iii) a 3′ flanking DNA for targeted chromosomal integration into the ER1 locus (SEQ ID NO:38). SEQ ID NO:2 contains (i) 5′ flanking DNA for targeted chromosomal integration into the ER1 locus (SEQ ID NO: 3); (ii) an expression cassette for the XPDH homolog from Lactobacillus rhamnosus, SEQ ID NO:5 encoding the amino acid sequence of SEQ ID NO:1, under the control of the PYK1 promoter of SEQ ID NO:4 and the PGD terminator of SEQ ID NO:16; and (iii) a 5′ portion of the G418 selectable marker (SEQ ID NO:51). The xylitol-phosphate dehydrogenase (XPDH) enzyme catalyzes the conversion of xylulose-5-phosphate and NADPH or NADH to xylitol 5-phosphate and NADP⁺ or NAD⁺, and this activity is referred to as “XPDH activity”. An “XPDH gene” refers to a polynucleotide sequence that encodes an enzyme with XPDH activity.

### Strain 1-3

Strain 1-2 was transformed as outlined above with SEQ ID NO:55 and SEQ ID NO: 56. The transformation fragment of SEQ ID NO:55 contained, in order, a 5′ ER3 flanking sequence (SEQ ID NO:31), a MpPYK1 promoter (SEQ ID NO:4), a gene encoding the M. pollinis RPE2 polypeptide of SEQ ID NO:54, a MpPYK terminator (SEQ ID NO:146), and a 5′ portion of a zeocin resistance gene expression cassette (SEQ ID NO:45). The transformation fragment of SEQ ID NO:56 contained, in order, a 3′ portion of a zeocin resistance gene expression cassette (SEQ ID NO:44), an MpTEF2 terminator (SEQ ID NO:26), and a 3′ ER3 flanking sequence (SEQ ID NO:41). Transformants were selected on PDA+zeocin selection plates and incubated at 35° C. for at least 2 days until transformants grow. Resulting transformants were streaked for single colony isolation on PDA+zeocin plates and single colonies were selected. Selected colonies were evaluated by colony PCR for integration of the gene encoding the M. pollinis RPE2 polypeptide. A PCR verified isolate was designated strain 1-3. The ribulose 5-phosphate epimerase (RPE) enzyme catalyzes the conversion of ribulose-5-phosphate to xylulose-5-phosphate, and this activity is referred to as “RPE” activity. An “RPE gene” refers to a polynucleotide sequence that encodes an enzyme with RPE activity, such as the RPE2 gene from M. pollinis which encodes the RPE enzyme RPE2.

### Strain 1-4

UV mutagenesis (using a Hoefer UV Crosslinker at an energy of 360 uJ/cm³) and selection of strain 1-3 was used to generate a Moniliella pollinis strain with reduced foaming during shake flask fermentation. Strains with low-foaming phenotypes were selected based on visual evaluation of foaming in a shake flask fermentation compared to foaming on the parent 1-3 strain. The resulting low-foaming strain, containing two copies of an exogenous polynucleotide sequence encoding the XPDH of SEQ ID NO:1 integrated at the ER1 locus and one copy of a polynucleotide sequence encoding the RPE of SEQ ID NO:54 integrated at the ER3 locus, was designated 1-4.

### Strain 1-5

Strain 1-4 was transformed with the Cre recombinase plasmid of SEQ ID NO:59 using the transformation method outlined above. The resulting transformants were evaluated by colony PCR for removal of the G418 and zeocin resistance selection marker. A PCR verified isolate was designated strain 1-5.

### Strain 1-6

Strain 1-5 was grown non-selectively on YPD plates to allow for the loss of the plasmid of SEQ ID NO:59. Biomass was struck for single colonies and evaluated by PCR to confirm loss of the plasmid. A PCR verified isolate was designated strain 1-6.

### Strain 1-7

Strain 1-6 was transformed with SEQ ID NO:57 and SEQ ID NO:55 as outlined above. SEQ ID NO:57 contains a 3′ portion of a zeocin resistance gene expression cassette (SEQ ID NO:168), an MpPGK1 promoter (SEQ ID NO:135), a polynucleotide sequence encoding the X5PP enzyme of SEQ ID NO:200, an Mp6PGD terminator (SEQ ID NO:140), and a 3′ ER3 flanking sequence (SEQ ID NO:165). Transformants were selected on PDA+zeocin selection plates and incubated at 35° C. for at least 2 days until transformants grow. Resulting transformants were streaked for single colony isolation on PDA+zeocin plates and single colonies were selected. Selected colonies were evaluated by colony PCR for integration of the M. pollinis X5PP gene at the ER3 locus. A PCR verified isolate was designated strain 1-7. The X5PP enzyme catalyzes the conversion of xylitol-5-phosphate to xylitol and phosphate in the presence of a divalent metal cation, for example, Mg²⁺, Mn²⁺, or Co²⁺, referred to as “X5PP activity.” An “X5PP gene” refers to a polynucleotide that encodes an enzyme with X5PP activity.

Strains 1-8a-e

Strain 1-7 was transformed with SEQ ID NO:75 and SEQ ID NO:58 using the transformation protocol outlined in Example 4. SEQ ID NO:75 contains a 5′ gpdIIB flanking sequence (SEQ ID NO:34), an MpPGK1 promoter (SEQ ID NO:11), a copy of the RCSR18717 gene on SEQ ID NO:61, a MpTDH3 terminator (SEQ ID NO:25), and a 5′ portion of the G418 resistance gene (SEQ ID NO:51). SEQ ID NO:58 contains a 3′ fragment of the G418 resistance gene (SEQ ID NO:48), an MpTEF1 terminator (SEQ ID NO:60), and a 3′ gpdIIB flanking sequence (SEQ ID NO:42). Transformants were selected on PDA+G418 selection plates and incubated as 35° C. for at least 2 days until transformants grew. Resulting transformants were streaked for single colony isolation of PDA+G418 plates and single colonies were selected. Selected colonies were evaluated by colony PCR for integration of the M. pollinis gene encoding the ER enzyme of SEQ ID NO:63. PCR verified sister isolates were designated strains 1-8a, 1-8b, 1-8c, 1-8d, and 1-8e

Strains 1-9a-b

Strain 1-7 was transformed with SEQ ID NO:70 using the transformation protocol outlined above and positive transformants were selected using nourseothricin selection plates. SEQ ID NO:70 contains a deletion construct to remove one copy of the RSCR18717 gene. Two PCR verified sister isolates, in which one copy of the RCSR18717 was knocked out, were designated strains 1-9a and 1-9b.

Strain 1-10a-d

Strain 1-9a was transformed with SEQ ID NO:71 using the transformation protocol outlined in Example 4. SEQ ID NO:71 contains a construct to loop out the zeocin and nourseothricin resistance selection markers. Two PCR verified sister isolates, in which the zeocin and nourseothricin resistance selection markers were removed, where designated strains 1-10a and 1-10b.

Strain 1-9b was transformed with SEQ ID NO:71 using the transformation protocol outlined in Example 4. SEQ ID NO:71 contains a construct to loop out the zeocin and nourseothricin resistance selection markers. Two PCR verified sister isolates, in which the zeocin and nourseothricin resistance selection markers were removed, were designated strains 1-10c and 1-10d.

Strains 1-11a-b

Strain 1-10c was transformed with SEQ ID NO:72 using the transformation protocol outlined in Example 4 and positive transformants were selected using geneticin (G418) selection plates. SEQ ID NO:72 contains a deletion construct to remove the second copy (i.e., only remaining copy) of the RCSR18717 gene. Two PCR verified sister isolates, in which both copies of the RCSR18717 gene was knocked out, were designated strains 1-11a and 1-11b.

Strains 1-12a-b

Strain 1-7 was transformed with SEQ ID NO:73 using the transformation protocol outlined in Example 4 and positive transformants were selected using nourseothricin selection plates. SEQ ID NO:73 contained a deletion construct to remove one copy of the RCSR26640 gene. Two PCR verified sister isolates, in which one copy of the RCSR26640 gene was knocked out, were designated strains 1-12a and 1-12b.

Strains 1-13a-d

Strain 1-12a was transformed with SEQ ID NO:71 using the transformation protocol outlined in Example 4. SEQ ID NO:71 contains a construct to loop out the zeocin and nourseothricin resistance selection markers. Two PCR verified sister isolates, in which the zeocin and nourseothricin resistance selection markers were removed, were designated strains 1-13a and 1-13b.

Strain 1-12b was transformed with SEQ ID NO:71 using the transformation protocol outlined in Example 4. SEQ ID NO:71 contains a construct to loop out the zeocin and nourseothricin resistance selection markers. Two PCR verified sister isolates, in which the zeocin and nourseothricin resistance selection markers were removed, were designated strains 1-13c and 1-13d.

Strain 1-14a-c

Strain 1-13c was transformed with SEQ ID NO:73 using the transformation protocol outlined in Example 4 and positive transformants were selected on nourseothricin selection plates. Three PCR verified sister isolate, in which both copies of the RCSR26640 gene were knocked out, were designated strains 1-14a, 1-14b, and 1-14c.

Strain 1-15a-b

Strain 1-7 was transformed with SEQ ID NO:74 using the transformation protocol outlined in Example 4 and positive transformants were selected using nourseothricin selection plates. SEQ ID NO:74 contained a deletion construct to remove the single copy of the RCSR11551 gene. Two PCR verified sister isolates, in which the single of the RCSR11551 gene was knocked out, were designated strains 1-15a and 1-15b.

Strain 1-16a-e

Strain 1-1 was transformed with SEQ ID NO:75 and SEQ ID NO:58 using the transformation method as outlined above. SEQ ID NO:75 contains a 5′ gpdIIB flanking sequence (SEQ ID NO:34), an MpPGK1 promoter (SEQ ID NO:11), a copy of the RCSR18717 gene of SEQ ID NO:61, a MpTDH3 terminator (SEQ ID NO:25), and a 5′ portion of the G418 resistance gene (SEQ ID NO:51). SEQ ID NO:58 contains a 3′ fragment of the G418 resistance gene (SEQ ID NO:48), an MpTEF1 terminator (SEQ ID NO:60), and a 3′ gpdIIB flanking sequence (SEQ ID NO:42). Transformants were selected on PDA+G418 selection plates and incubated as 35° C. for at least 2 days until transformants grew. Resulting transformants were streaked for single colony isolation of PDA+G418 plates and single colonies were selected. Selected colonies were evaluated by colony PCR for integration of the M. pollinis gene encoding the ER enzyme of SEQ ID NO:63. PCR verified sister isolates were designated strains 1-16a, 1-16b, 1-16c, 1-16d, and 1-16e.

TABLE 5
Summary of Strains

| | Parent | |
|---|---|---|
| Strain | Strain | Notes |
| 1-2 | 1-1 | Strain 1-1 + 2x L. rhamnosus XPDH at ER1 |
| 1-3 | 1-2 | Strain 1-1 + 2x L. rhamnosus XPDH at ER1 + 1X M. pollinis RPE2 at |
| | | ER3 |
| 1-4 | 1-3 | Strain 1-1 + 2x L. rhamnosus XPDH at ER1 + 1X M. pollinis RPE2 at |
| | | ER3 and UV mutagenized (with low foaming phenotype relative to 1-3) |
| 1-5 | 1-4 | Strain 1-1 + 2x L. rhamnosus XPDH at ER1 + 1X M. pollinis RPE2 at |
| | | ER3, UV mutagenized, and G418 and zeocin resistance marker loop out |
| | | (with CRE recombinase plasmid) |
| 1-6 | 1-5 | Strain 1-1 + 2x L. rhamnosus XPDH at ER1 + 1X M. pollinis RPE2 at |
| | | ER3, UV mutagenized, and G418 and zeocin resistance marker loop out |
| | | (loss of CRE recombinase plasmid) |
| 1-7 | 1-6 | Strain 1-1 + 2x L. rhamnosus XPDH at ER1 + 2X M. pollinis RPE2 at |
| | | ER3, UV mutagenized, +2X M. pollinis X5PP at ER3 |
| 1-8a-e | 1-7 | 1-1 + 2x L. rhamnosus XPDH at ER1 + 2X M. pollinis RPE2 at ER3, UV |
| | | mutagenized, +2X M. pollinis X5PP at ER3, +1X RCSR18717 at |
| | | gpdIIB |
| 1-9a-b | 1-7 | 1-1 + 2x L. rhamnosus XPDH at ER1 + 2X M. pollinis RPE2 at ER3, UV |
| | | mutagenized, +2X M. pollinis X5PP at ER3-1X RCSR18717 |
| 1-10a-b | 1-9a | 1-1 + 2x L. rhamnosus XPDH at ER1 + 2X M. pollinis RPE2 at ER3, UV |
| 1-10c-d | 1-9b | mutagenized, +2X M. pollinis X5PP at ER3, −1X RCSR18717, NAT |
| | | and zeocin resistance marker loop out |
| 1-11a-b | 1-10c | 1-1 + 2x L. rhamnosus XPDH at ER1 + 2X M. pollinis RPE2 at ER3, UV |
| | | mutagenized, +2X M. pollinis X5PP at ER3-2X RCSR18717 |
| 1-12a-b | 1-7 | 1-1 + 2x L. rhamnosus XPDH at ER1 + 2X M. pollinis RPE2 at ER3, UV |
| | | mutagenized, +2X M. pollinis X5PP at ER3-1X RCSR26640 |
| 1-13a-b | 1-12a | 1-1 + 2x L. rhamnosus XPDH at ER1 + 2X M. pollinis RPE2 at ER3, UV |
| 1-13c-d | 1-12b | mutagenized, +2X M. pollinis X5PP at ER3-1X RCSR26640, NAT and |
| | | zeocin resistance marker loop out |
| 1-14a-c | 1-13c | 1-1 + 2x L. rhamnosus XPDH at ER1 + 2X M. pollinis RPE2 at ER3, UV |
| | | mutagenized, +2X M. pollinis X5PP at ER3-2X RCSR26640 |
| 1-15a-b | | 1-1 + 2x L. rhamnosus XPDH at ER1 + 2X M. pollinis RPE2 at ER3, UV |
| | | mutagenized, +2X M. pollinis X5PP at ER3-1X RCSR11551 |
| | | (RCSR11551 is haploid so this is a complete knock out) |
| 1-16a-e | 1-1 | 1-1 + 1X RCSR18717 at gpdIIB |

### Example 3: Shake Flask Fermentation Assay

Strains 1-7, 1-12a, 1-12b, 1-9a, 1-9b, 1-15a, and 1-15b were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

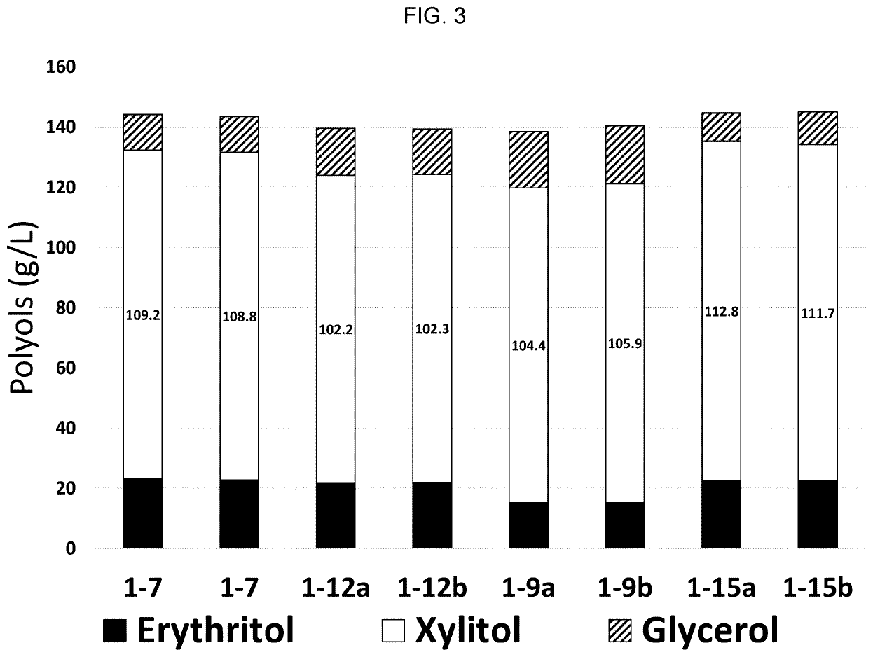

A 250 ml non-baffled flask containing 20 mL production medium (Table 6) was inoculated with 0.4 mL of the seed culture to form the production culture. The production culture was incubated at 35° C. and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Results are shown in Tables 7 and 8 and results for the 72-hour time point are also shown in FIG. 3.

Overall, strains 1-9a and 1-9b showed the greatest reduction in erythritol production (32%) relative to the parent (1-7).

TABLE 6
Production Medium

| Component | Concentration (units) |
|---|---|
| Glucose | 300 (g/L) |
| KH2PO4 | 1.27 (g/L) |
| (NH4)2HPO4 | 0.13 (g/L) |
| (NH4)2SO4 | 1.80 (g/L) |
| Urea | 2.85 (g/L) |
| Citric Acid | 200.0 (mg/L) |
| MgSO4—7H2O | 690.2 (mg/L) |
| FeSO4—7H2O | 32.35 (mg/L) |
| ZnSO4—7H2O | 4.40 (mg/L) |
| MnSO4—H2O | 0.53 (mg/L) |
| CuSO4—5H2O | 0.45 (mg/L) |
| CaCl2—2H2O | 91.60 (mg/L) |
| Na2SO4 | 154.50 (mg/L) |
| Thiamine-HCl | 10 (mg/L) |
| Choline-Cl | 100 (mg/L) |
| Antifoam CF-32 | 1.00 (g/L) |

TABLE 7

| | Time | | | | | | |
|---|---|---|---|---|---|---|---|
| | Point | Fermentation Broth Analyte (g/L) | | | | | |
| Strain | (h) | Glucose | Ribitol | Xylitol | Erythritol | Glycerol | Ethanol |
| 1-7 | 24 | 276.62 | 0.03 | 1.55 | 1.06 | 4.32 | 0.13 |
| 1-7 | 24 | 279.28 | 0.00 | 1.58 | 1.07 | 4.56 | 0.27 |
| 1-12a | 24 | 279.49 | 0.03 | 1.42 | 0.93 | 3.79 | 0.24 |
| 1-12b | 24 | 281.54 | 0.04 | 1.43 | 0.95 | 3.72 | 0.21 |
| 1-9a | 24 | 281.47 | 0.04 | 1.39 | 0.57 | 3.76 | 0.26 |
| 1-9b | 24 | 282.38 | 0.05 | 1.51 | 0.66 | 4.17 | 0.27 |
| 1-15a | 24 | 278.83 | 0.01 | 1.51 | 1.05 | 4.59 | 0.28 |
| 1-15b | 24 | 283.19 | 0.06 | 1.56 | 0.99 | 4.08 | 0.34 |
| 1-7 | 72 | 0.26 | 0.35 | 101.70 | 22.48 | 18.11 | 29.13 |
| 1-7 | 72 | 0.45 | 0.35 | 102.00 | 22.30 | 18.23 | 29.91 |
| 1-12a | 72 | 2.35 | 0.33 | 99.01 | 21.90 | 16.78 | 31.36 |
| 1-12b | 72 | 2.44 | 0.33 | 98.43 | 21.85 | 16.15 | 31.34 |
| 1-9a | 72 | 11.45 | 0.32 | 90.94 | 15.29 | 24.88 | 32.68 |
| 1-9b | 72 | 4.15 | 0.34 | 97.14 | 15.12 | 24.03 | 32.32 |
| 1-15a | 72 | 1.42 | 0.35 | 101.33 | 22.17 | 17.97 | 31.60 |
| 1-15b | 72 | 0.26 | 0.35 | 101.70 | 22.48 | 18.11 | 29.13 |
| | Time | | | | | | |
| | Point | Fermentation Broth Analyte (g/L) | | | | | |
| Strain | (h) | Glucose | Ribitol | Xylitol | Erythritol | Glycerol | Ethanol |
| 1-7 | 48 | 168.65 | 0.14 | 26.77 | 7.78 | 21.27 | 16.89 |
| 1-7 | 48 | 167.61 | 0.13 | 26.71 | 7.73 | 20.96 | 16.70 |
| 1-12a | 48 | 170.31 | 0.11 | 25.84 | 7.33 | 19.58 | 16.73 |
| 1-12b | 48 | 171.64 | 0.12 | 25.91 | 7.24 | 19.16 | 16.92 |
| 1-9a | 48 | 194.37 | 0.10 | 18.89 | 4.26 | 23.00 | 11.75 |
| 1-9b | 48 | 182.43 | 0.11 | 22.31 | 4.88 | 23.50 | 13.44 |
| 1-15a | 48 | 173.82 | 0.12 | 25.90 | 7.07 | 19.56 | 17.24 |
| 1-15b | 48 | 182.43 | 0.11 | 22.15 | 6.36 | 20.95 | 14.68 |
| 1-7 | 96 | 0.02 | 0.40 | 109.24 | 23.11 | 11.82 | 16.95 |
| 1-7 | 96 | 0.02 | 0.40 | 108.83 | 22.82 | 11.91 | 19.91 |
| 1-12a | 96 | 0.58 | 0.33 | 102.18 | 21.84 | 15.61 | 22.72 |
| 1-12b | 96 | 0.55 | 0.33 | 102.34 | 22.00 | 15.05 | 22.11 |
| 1-9a | 96 | 0.02 | 0.38 | 104.40 | 15.44 | 18.71 | 23.48 |
| 1-9b | 96 | 0.02 | 0.38 | 105.87 | 15.41 | 19.11 | 23.08 |
| 1-15a | 96 | 0.02 | 0.42 | 112.84 | 22.49 | 9.36 | 19.79 |
| 1-15b | 96 | 0.01 | 0.44 | 111.70 | 22.53 | 10.82 | 19.67 |

TABLE 8

| Strain | 1-7 | 1-7 | 1-12a | 1-12b | 1-9a | 1-9b | 1-15a | 1-15b |
|---|---|---|---|---|---|---|---|---|
| Xylitol | 36.5 | 36.4 | 34.2 | 34.3 | 34.9 | 35.4 | 37.7 | 37.4 |
| Yield (%) | | | | | | | | |

### Example 4: Shake Flask Fermentation Assay

Strains 1-7, 1-9a, 1-9b, 1-10a, 1-10b, 1-10c, 1-10d, 1-12a, 1-12b, 1-13a, 1-13b, 1-13c, and 1-13d were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

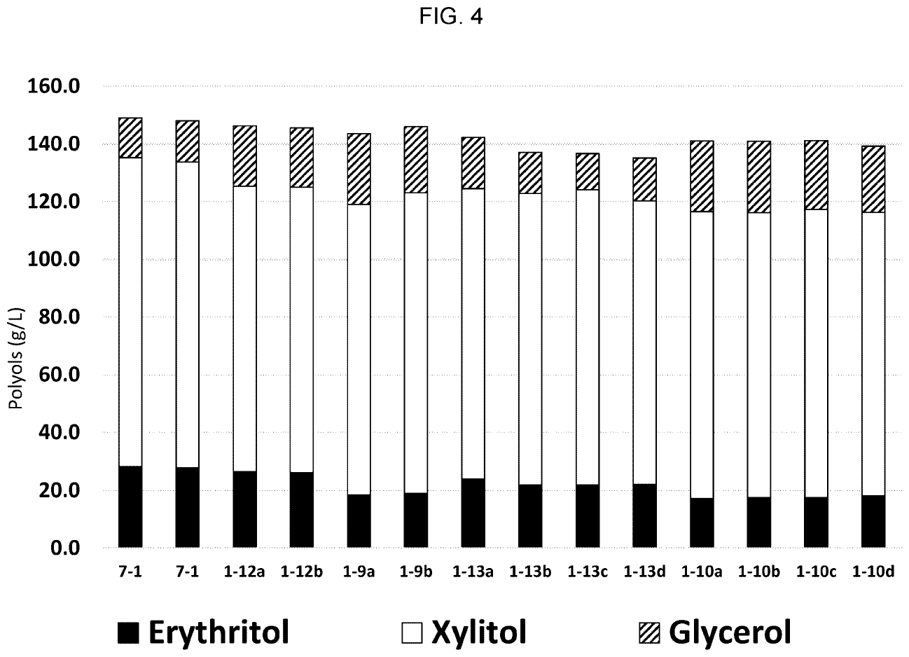

A 250 ml non-baffled flask containing 20 mL production medium (Table 6) was inoculated with 0.4 mL of the seed culture to form the production culture. The production culture was incubated at 35° C. and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Results are shown in Table 9 and results for the 96-hour time point are also shown in FIG. 4.

Overall, strains 1-10a-d showed a decrease in erythritol relative to the grandparent strain 1-7. The erythritol production in strains 1-10a-d is similar to strains 1-9a-b, and all six of these strains have a knockout of one copy of the RCSR18717 gene. Strains 1-13a-d also show a slight decrease in erythritol production relative to the grandparent strain 6-b and this decrease is greater than the decrease seen in parent strains 1-12a-b.

TABLE 9

| | Time | | | | | | |
|---|---|---|---|---|---|---|---|
| | Point | Fermentation Broth Analyte (g/L) | | | | | |
| Strain | (h) | Glucose | Ribitol | Xylitol | Erythritol | Glycerol | Ethanol |
| 1-7 | 24 | 284.49 | 0.00 | 1.60 | 1.38 | 5.94 | 0.00 |
| 1-7 | 24 | 285.71 | 0.00 | 1.55 | 1.26 | 5.80 | 0.00 |
| 1-12a | 24 | 286.64 | 0.00 | 1.75 | 1.25 | 5.39 | 0.37 |
| 1-12b | 24 | 284.64 | 0.00 | 1.58 | 1.19 | 5.19 | 0.41 |
| 1-9a | 24 | 286.73 | 0.00 | 1.51 | 0.63 | 5.08 | 0.31 |
| 1-9b | 24 | 287.92 | 0.00 | 1.61 | 0.83 | 5.70 | 0.00 |
| 1-13a | 24 | 286.22 | 0.00 | 1.37 | 1.43 | 5.97 | 0.37 |
| 1-13b | 24 | 285.35 | 0.00 | 1.60 | 1.38 | 5.01 | 0.60 |
| 1-13c | 24 | 283.53 | 0.00 | 1.55 | 1.27 | 5.00 | 0.65 |
| 1-13d | 24 | 282.01 | 0.00 | 1.48 | 1.51 | 6.32 | 0.78 |
| 1-10a | 24 | 284.74 | 0.00 | 1.33 | 1.01 | 6.66 | 0.50 |
| 1-10b | 24 | 283.92 | 0.00 | 1.43 | 1.03 | 6.77 | 0.47 |
| 1-10c | 24 | 284.15 | 0.00 | 1.37 | 1.01 | 6.39 | 0.58 |
| 1-10d | 24 | 283.60 | 0.00 | 0.64 | 1.45 | 6.98 | 0.47 |
| 1-7 | 72 | 2.36 | 0.39 | 96.69 | 26.61 | 21.94 | 30.73 |
| 1-7 | 72 | 2.96 | 0.24 | 95.40 | 25.82 | 21.55 | 31.67 |
| 1-12a | 72 | 10.79 | 0.20 | 90.53 | 24.88 | 21.25 | 31.38 |
| 1-12b | 72 | 11.16 | 0.21 | 90.96 | 24.87 | 20.80 | 31.16 |
| 1-9a | 72 | 26.78 | 0.20 | 80.01 | 17.14 | 27.68 | 32.23 |
| 1-9b | 72 | 14.37 | 0.22 | 87.90 | 18.32 | 27.00 | 32.15 |
| 1-13a | 72 | 18.57 | 0.34 | 87.63 | 22.02 | 18.55 | 34.04 |
| 1-13b | 72 | 5.59 | 0.51 | 94.75 | 20.98 | 14.88 | 38.43 |
| 1-13c | 72 | 4.87 | 0.55 | 95.52 | 21.04 | 14.91 | 37.95 |
| 1-13d | 72 | 5.79 | 0.61 | 91.55 | 21.64 | 15.15 | 36.15 |
| 1-10a | 72 | 16.38 | 0.48 | 83.22 | 17.65 | 27.41 | 33.95 |
| 1-10b | 72 | 21.86 | 0.42 | 82.14 | 16.30 | 26.83 | 34.26 |
| 1-10c | 72 | 23.93 | 0.35 | 81.69 | 16.07 | 26.05 | 33.20 |
| 1-10d | 72 | 13.24 | 0.53 | 85.95 | 17.15 | 24.10 | 35.44 |
| | Time | | | | | | |
| | Point | Fermentation Broth Analyte (g/L) | | | | | |
| Strain | (h) | Glucose | Ribitol | Xylitol | Erythritol | Glycerol | Ethanol |
| 1-7 | 48 | 157.02 | 0.12 | 29.74 | 10.52 | 22.33 | 18.09 |
| 1-7 | 48 | 162.85 | 0.10 | 28.90 | 10.27 | 22.07 | 18.29 |
| 1-12a | 48 | 168.67 | 0.09 | 25.91 | 9.10 | 22.01 | 16.74 |
| 1-12b | 48 | 167.70 | 0.11 | 25.36 | 9.09 | 21.91 | 16.48 |
| 1-9a | 48 | 190.79 | 0.07 | 19.77 | 5.48 | 25.33 | 12.07 |
| 1-9b | 48 | 179.74 | 0.07 | 23.34 | 6.39 | 25.08 | 14.47 |
| 1-13a | 48 | 162.71 | 0.13 | 26.15 | 8.56 | 19.62 | 20.20 |
| 1-13b | 48 | 155.07 | 0.19 | 29.88 | 9.45 | 16.30 | 23.33 |
| 1-13c | 48 | 155.34 | 0.16 | 29.76 | 9.04 | 17.07 | 21.92 |
| 1-13d | 48 | 144.68 | 0.18 | 30.68 | 10.11 | 17.89 | 24.10 |
| 1-10a | 48 | 168.80 | 0.16 | 22.75 | 6.51 | 26.09 | 18.39 |
| 1-10b | 48 | 168.66 | 0.13 | 22.90 | 6.63 | 25.35 | 18.20 |
| 1-10c | 48 | 165.33 | 0.16 | 23.50 | 6.91 | 25.31 | 18.39 |
| 1-10d | 48 | 151.63 | 0.14 | 26.56 | 8.41 | 23.61 | 22.48 |
| 1-7 | 96 | 0.06 | 0.34 | 107.17 | 28.11 | 13.72 | 16.54 |
| 1-7 | 96 | 0.05 | 0.36 | 105.92 | 27.76 | 14.34 | 17.60 |
| 1-12a | 96 | 2.22 | 0.33 | 98.88 | 26.42 | 21.01 | 19.94 |
| 1-12b | 96 | 2.74 | 0.28 | 98.84 | 26.13 | 20.56 | 20.39 |
| 1-9a | 96 | 0.13 | 0.35 | 100.68 | 18.37 | 24.55 | 22.00 |
| 1-9b | 96 | 0.07 | 0.40 | 104.16 | 18.92 | 22.85 | 20.21 |
| 1-13a | 96 | 0.74 | 0.59 | 100.70 | 23.82 | 17.65 | 20.14 |
| 1-13b | 96 | 0.30 | 0.62 | 101.13 | 21.76 | 14.16 | 23.92 |
| 1-13c | 96 | 0.08 | 0.63 | 102.36 | 21.77 | 12.54 | 25.00 |
| 1-13d | 96 | 0.75 | 0.65 | 98.27 | 22.00 | 14.91 | 23.87 |
| 1-10a | 96 | 0.07 | 0.62 | 99.41 | 17.17 | 24.46 | 21.15 |
| 1-10b | 96 | 0.10 | 0.61 | 98.69 | 17.50 | 24.72 | 21.86 |
| 1-10c | 96 | 0.13 | 0.63 | 99.68 | 17.55 | 23.88 | 22.90 |
| 1-10d | 96 | 0.17 | 0.61 | 98.35 | 17.98 | 22.91 | 23.37 |

### Example 5: Shake Flask Fermentation Assay

Strains 1-7, 1-13c, 1-14a, 1-14b, and 1-14c were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

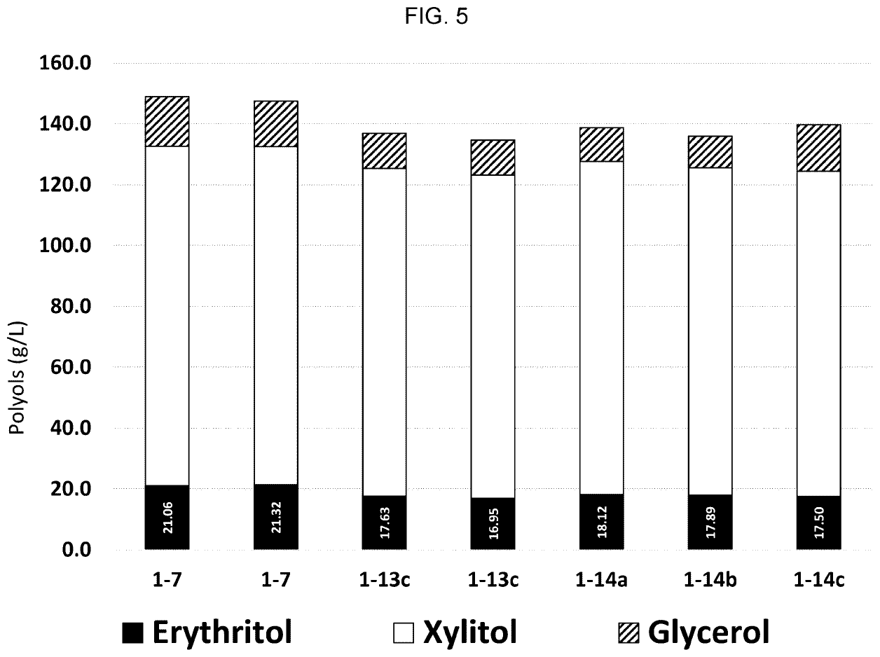

A 250 ml non-baffled flask containing 20 mL production medium (Table 6) was inoculated with 0.4 mL of the seed culture to form the production culture. The production culture was incubated at 35° C. and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Results are shown in Table 10 and results for the 96-hour time point are also shown in FIG. 5.

Overall, strains 1-13c, 1-14a, 1-14b, and 1-14c showed reduced erythritol production relative to strain 1-7. However, knockout of the second allele of RCSR26640 (i.e., strains 1-14a, 1-14b, and 1-14c) did not further decrease erythritol production beyond the single knock out strains (1-13c).

TABLE 10

| | Time | | | | | | |
|---|---|---|---|---|---|---|---|
| | Point | Fermentation Broth Analyte (g/L) | | | | | |
| Strain | (h) | Glucose | Ribitol | Xylitol | Erythritol | Glycerol | Ethanol |
| 1-7 | 24 | 281.32 | 0.06 | 2.00 | 1.78 | 7.71 | 0.30 |
| 1-7 | 24 | 276.69 | 0.12 | 2.16 | 1.78 | 7.83 | 0.54 |
| 1-13c | 24 | 274.89 | 0.09 | 2.33 | 2.03 | 7.97 | 1.10 |
| 1-13c | 24 | 280.60 | 0.14 | 1.75 | 1.61 | 6.05 | 0.67 |
| 1-14a | 24 | 280.22 | 0.11 | 1.76 | 1.68 | 6.70 | 0.64 |
| 1-14b | 24 | 275.76 | 0.14 | 2.16 | 1.84 | 7.27 | 0.91 |
| 1-14c | 24 | 275.04 | 0.11 | 2.14 | 1.91 | 7.18 | 0.99 |
| 1-7 | 72 | 4.32 | 0.28 | 94.16 | 26.60 | 23.15 | 31.19 |
| 1-7 | 72 | 4.96 | 0.27 | 92.17 | 27.42 | 23.26 | 30.66 |
| 1-13c | 72 | 1.34 | 0.60 | 96.36 | 23.02 | 15.57 | 34.06 |
| 1-13c | 72 | 4.53 | 0.53 | 92.37 | 22.95 | 15.27 | 37.31 |
| 1-14a | 72 | 3.06 | 0.49 | 95.14 | 24.08 | 13.94 | 35.16 |
| 1-14b | 72 | 2.09 | 0.56 | 95.01 | 23.97 | 14.75 | 34.70 |
| 1-14c | 72 | 2.51 | 0.50 | 95.23 | 23.68 | 13.44 | 36.24 |
| | Time | | | | | | |
| | Point | Fermentation Broth Analyte (g/L) | | | | | |
| Strain | (h) | Glucose | Ribitol | Xylitol | Erythritol | Glycerol | Ethanol |
| 1-7 | 48 | 164.97 | 0.10 | 27.56 | 10.14 | 22.88 | 17.67 |
| 1-7 | 48 | 163.13 | 0.11 | 27.72 | 10.27 | 23.25 | 17.31 |
| 1-13c | 48 | 138.80 | 0.20 | 33.07 | 10.71 | 19.22 | 24.24 |
| 1-13c | 48 | 158.76 | 0.20 | 27.93 | 9.10 | 17.71 | 22.30 |
| 1-14a | 48 | 152.29 | 0.20 | 29.95 | 9.90 | 17.70 | 23.06 |
| 1-14b | 48 | 143.77 | 0.24 | 31.32 | 10.56 | 19.16 | 23.98 |
| 1-14c | 48 | 144.19 | 0.19 | 31.46 | 10.46 | 17.68 | 24.32 |
| 1-7 | 96 | 0.06 | 0.33 | 111.56 | 21.06 | 16.27 | 16.88 |
| 1-7 | 96 | 0.08 | 0.33 | 111.17 | 21.32 | 16.55 | 16.53 |
| 1-13c | 96 | 0.03 | 0.71 | 107.73 | 17.63 | 11.72 | 20.31 |
| 1-13c | 96 | 0.06 | 0.70 | 106.23 | 16.95 | 11.55 | 24.64 |
| 1-14a | 96 | 0.05 | 0.70 | 109.51 | 18.12 | 9.36 | 19.04 |
| 1-14b | 96 | 0.08 | 0.68 | 107.67 | 17.89 | 11.11 | 22.25 |
| 1-14c | 96 | 0.09 | 0.68 | 106.91 | 17.50 | 10.35 | 23.91 |

### Example 6: Shake Flask Fermentation Assay

Strains 1-7, 1-10a, 1-10c, and 1-11a-b were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

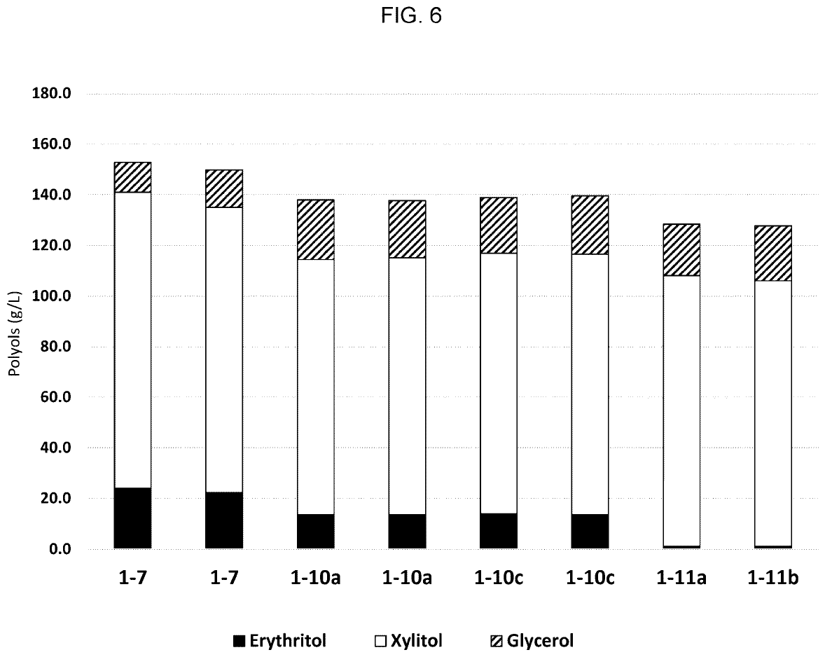

A 250 ml non-baffled flask containing 20 mL production medium (Table 6) was inoculated with 0.4 mL of the seed culture to form the production culture. The production culture was incubated at 35° C. and 250 rpm. Samples were taken from the production culture after 24, 48, 72, and 96 hours of incubation. Samples were analyzed for glucose, ribitol, xylitol, erythritol, glycerol, and ethanol by high performance liquid chromatography with refractive index detector. Results are shown in Table 11. Results for the 96-hour time point comparing strains 1-10a, 1-10c, 1-11a, and 1-11b are shown in FIG. 6

Overall, strains 1-11a and 1-11b showed significant reduction in erythritol production, with the erythritol titer at 96 hours reduced to about 1.0 g/L. The two allele knock out of the RCSR18717 gene in these strains shows significant erythritol reduction relative to both the single knock out strains (1-10a and 1-10c) and to the grandparent strain (1-7) which includes both wild-type alleles of RCSR18717.

TABLE 11

| | Time | | | | | | |
|---|---|---|---|---|---|---|---|
| | Point | Fermentation Broth Analyte (g/L) | | | | | |
| Strain | (h) | Glucose | Ribitol | Xylitol | Erythritol | Glycerol | Ethanol |
| 1-7 | 24 | 286.01 | 0.08 | 2.02 | 1.22 | 5.60 | 0.47 |
| 1-7 | 24 | 284.78 | 0.08 | 2.35 | 1.31 | 6.19 | 0.66 |
| 1-10a | 24 | 285.37 | 0.07 | 1.88 | 0.95 | 6.27 | 0.96 |
| 1-10a | 24 | 283.95 | 0.07 | 1.91 | 0.98 | 6.51 | 0.96 |
| 1-10c | 24 | 283.47 | 0.07 | 1.77 | 0.99 | 6.40 | 0.87 |
| 1-10c | 24 | 284.77 | 0.09 | 1.73 | 0.98 | 6.46 | 0.87 |
| 1-11a | 24 | 287.66 | 0.08 | 1.71 | 0.04 | 4.88 | 1.06 |
| 1-11b | 24 | 286.59 | 0.06 | 1.86 | 0.04 | 5.15 | 1.13 |
| 1-7 | 72 | 4.63 | 0.28 | 102.72 | 22.85 | 21.71 | 28.57 |
| 1-7 | 72 | 4.59 | 0.25 | 101.92 | 21.41 | 22.81 | 30.89 |
| 1-10a | 72 | 30.34 | 0.50 | 83.75 | 13.15 | 26.15 | 35.14 |
| 1-10a | 72 | 26.50 | 0.49 | 84.76 | 13.04 | 24.82 | 35.27 |
| 1-10c | 72 | 28.75 | 0.50 | 84.97 | 13.06 | 24.59 | 34.03 |
| 1-10c | 72 | 28.51 | 0.51 | 85.88 | 13.13 | 26.11 | 33.22 |
| 1-11a | 72 | 61.22 | 0.42 | 76.53 | 0.66 | 21.69 | 33.27 |
| 1-11b | 72 | 63.59 | 0.35 | 75.71 | 0.62 | 22.96 | 31.56 |
| | Time | | | | | | |
| | Point | Fermentation Broth Analyte (g/L) | | | | | |
| Strain | (h) | Glucose | Ribitol | Xylitol | Erythritol | Glycerol | Ethanol |
| 1-7 | 48 | 164.38 | 0.12 | 31.21 | 8.03 | 21.35 | 17.94 |
| 1-7 | 48 | 159.79 | 0.14 | 31.96 | 8.34 | 22.55 | 18.28 |
| 1-10a | 48 | 172.15 | 0.18 | 24.29 | 5.27 | 24.56 | 18.29 |
| 1-10a | 48 | 168.92 | 0.18 | 25.42 | 5.42 | 23.92 | 19.14 |
| 1-10c | 48 | 168.55 | 0.17 | 25.37 | 5.51 | 23.82 | 19.52 |
| 1-10c | 48 | 169.29 | 0.17 | 25.43 | 5.55 | 25.00 | 18.71 |
| 1-11a | 48 | 190.43 | 0.16 | 21.70 | 0.29 | 20.62 | 17.88 |
| 1-11b | 48 | 188.54 | 0.14 | 22.41 | 0.29 | 21.55 | 17.22 |
| 1-7 | 96 | 0.01 | 0.46 | 117.06 | 23.94 | 11.81 | 12.09 |
| 1-7 | 96 | 0.04 | 0.46 | 112.73 | 22.26 | 14.85 | 18.21 |
| 1-10a | 96 | 0.43 | 0.80 | 100.91 | 13.50 | 23.53 | 26.35 |
| 1-10a | 96 | 0.35 | 0.80 | 101.56 | 13.57 | 22.50 | 25.89 |
| 1-10c | 96 | 0.33 | 0.78 | 102.95 | 13.89 | 21.96 | 25.08 |
| 1-10c | 96 | 0.29 | 0.79 | 102.96 | 13.56 | 22.98 | 23.81 |
| 1-11a | 96 | 1.96 | 0.81 | 107.01 | 1.04 | 20.33 | 30.61 |
| 1-11b | 96 | 6.92 | 0.73 | 105.02 | 1.01 | 21.73 | 29.35 |

### Example 7: Shake Flask Fermentation Assay

Strains 1-7 and 1-8a-e were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

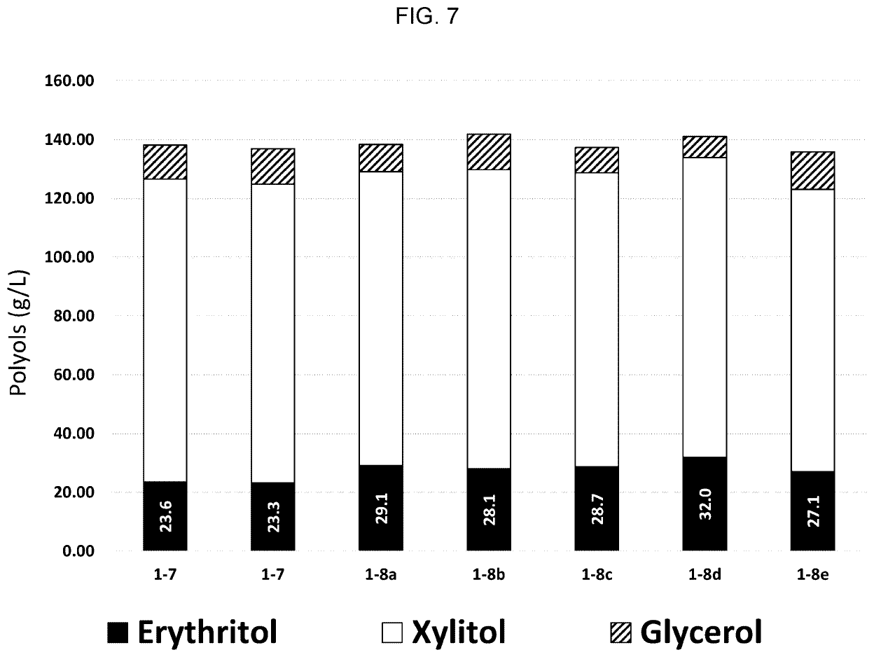

A 250 ml non-baffled flask containing 20 mL production medium (Table 6) was inoculated with 0.4 mL of the seed culture to form the production culture. The production culture was incubated at 35° C. and 250 rpm. Samples were taken from the production culture after 72 and 96 hours of incubation. Samples were analyzed for glucose, ribitol, xylitol, erythritol, glycerol, and ethanol by high performance liquid chromatography with refractive index detector. Results are shown in Table 12. Results for the 96-hour time point are shown in FIG. 7. Overall, strains 1-8a-e, which are engineered to overexpress the RCSR18717 gene, showed an increase in erythritol production relative to control strain 1-7, which has wild-type levels of RCSR18717 expression. Strains 1-8a-e showed an approximately 25% increase in erythritol titer relative to strain 1-7.

TABLE 12

| | Time | | | | | | |
|---|---|---|---|---|---|---|---|
| | Point | Fermentation Broth Analyte (g/L) | | | | | |
| Strain | (h) | Glucose | Ribitol | Xylitol | Erythritol | Glycerol | Ethanol |
| 1-7 | 72 | 2.02 | 0.25 | 91.59 | 22.56 | 18.46 | 30.47 |
| 1-7 | 72 | 2.98 | 0.34 | 91.51 | 22.05 | 18.54 | 31.40 |
| 1-8a | 72 | 0.70 | 0.31 | 93.31 | 27.79 | 14.52 | 30.92 |
| 1-8b | 72 | 4.26 | 0.32 | 91.81 | 27.32 | 15.46 | 30.62 |
| 1-8c | 72 | 0.68 | 0.32 | 93.48 | 27.71 | 13.57 | 32.05 |
| 1-8d | 72 | 0.32 | 0.34 | 93.09 | 30.28 | 15.02 | 28.44 |
| 1-8c | 72 | 0.67 | 0.30 | 90.28 | 25.86 | 18.34 | 30.23 |
| | Time | | | | | | |
| | Point | Fermentation Broth Analyte (g/L) | | | | | |
| Strain | (h) | Glucose | Ribitol | Xylitol | Erythritol | Glycerol | Ethanol |
| 1-7 | 96 | 0.07 | 0.34 | 103.06 | 23.56 | 11.57 | 16.27 |
| 1-7 | 96 | 0.02 | 0.47 | 101.55 | 23.27 | 12.04 | 19.83 |
| 1-8a | 96 | 0.03 | 0.44 | 99.97 | 29.12 | 9.31 | 19.48 |
| 1-8b | 96 | 0.05 | 0.36 | 101.72 | 28.08 | 12.11 | 20.42 |
| 1-8c | 96 | 0.04 | 0.37 | 100.07 | 28.68 | 8.55 | 19.51 |
| 1-8d | 96 | 0.04 | 0.42 | 102.00 | 31.97 | 7.05 | 16.15 |
| 1-8c | 96 | 0.04 | 0.40 | 96.03 | 27.09 | 12.74 | 18.77 |

### Example 8: Shake Flask Fermentation Assay

Strains 1-7 and 1-16a-e were run in shake flasks to assess glucose consumption as well as ribitol, xylitol, erythritol, glycerol, and ethanol production.

Strains were streaked out for biomass growth on YPD plates (bacteriological peptone 20 g/L, yeast extract 10 g/L, glucose 20 g/L, and agar 15 g/L) and incubated at 30° C. for 48-72 hours. Cells from the incubated YPD plates were scraped into 40 mL rich medium (170 g/L glucose, 10 g/L yeast extract) in a 250 mL non-baffled flask. Cells were incubated at 30° C. and 250 rpm until the optical density (OD600) reached 15-20 to form the seed culture. Optical density is measured at a wavelength of 600 nm with a 1 cm path length cuvette using a model Genesys20 spectrophotometer (Thermo Scientific). The seed culture reached an OD600 between 15-20 in about 32-50 hours.

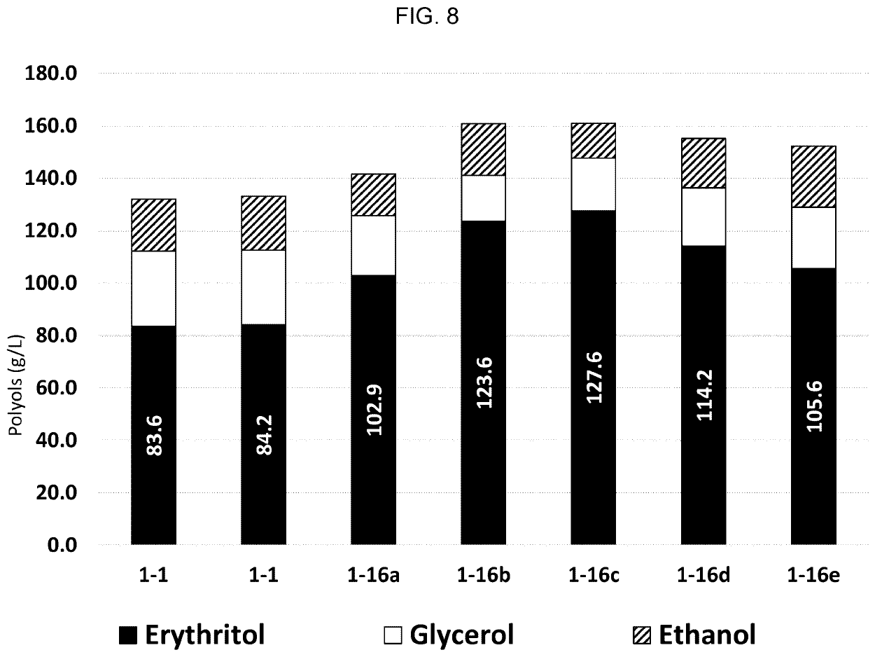

A 250 ml non-baffled flask containing 20 mL production medium (Table 6) was inoculated with 0.4 mL of the seed culture to form the production culture. The production culture was incubated at 35° C. and 250 rpm. Samples were taken from the production culture after 24, 48, 72, and 100 hours of incubation. Samples were analyzed for glucose, ribitol, xylitol, erythritol, glycerol, and ethanol by high performance liquid chromatography with refractive index detector. Results are shown in Table 13. Results for the 100-hour time point are shown in FIG. 8. Overall, strains 1-16a-e, which are engineered to overexpress the RCSR18717 gene, showed an increase in erythritol production relative to the wild-type strain 1-1, which has wild-type levels of RCSR18717 expression. Erythritol yield was also higher in strains 1-16a-e than strain 11-1 (Table 14).

TABLE 13

| | Time | | | | | | |
|---|---|---|---|---|---|---|---|
| | Point | Fermentation Broth Analyte (g/L) | | | | | |
| Strain | (h) | Glucosc | Ribitol | Xylitol | Erythritol | Glycerol | Ethanol |
| 1-1 | 24 | 279.52 | 0.06 | n.a. | 3.72 | 6.23 | 0.65 |
| 1-1 | 24 | 283.61 | 0.08 | n.a. | 3.91 | 6.56 | 0.73 |
| 1-16a | 24 | 282.07 | 0.07 | n.a. | 4.18 | 5.58 | 0.52 |
| 1-16b | 24 | 281.40 | 0.12 | n.a. | 4.65 | 5.62 | 0.62 |
| 1-16c | 24 | 283.32 | 0.13 | n.a. | 4.79 | 6.03 | 0.61 |
| 1-16d | 24 | 281.89 | 0.05 | n.a. | 4.18 | 5.76 | 0.64 |
| 1-16e | 24 | 284.87 | 0.06 | n.a. | 3.93 | 4.98 | 0.20 |
| 1-1 | 72 | 48.22 | 0.61 | 0.07 | 74.01 | 27.56 | 29.59 |
| 1-1 | 72 | 46.21 | 0.63 | 0.07 | 74.93 | 27.27 | 29.98 |
| 1-16a | 72 | 36.94 | 1.22 | 0.00 | 93.29 | 22.59 | 27.54 |
| 1-16b | 72 | 11.84 | 2.91 | 0.00 | 108.84 | 23.90 | 30.52 |
| 1-16c | 72 | 6.33 | 3.15 | 0.00 | 113.65 | 25.07 | 27.92 |
| 1-16d | 72 | 23.97 | 1.29 | 0.00 | 99.60 | 23.27 | 29.54 |
| 1-16e | 72 | 13.76 | 1.41 | 0.06 | 98.49 | 24.72 | 34.12 |
| | Time | | | | | | |
| | Point | Fermentation Broth Analyte (g/L) | | | | | |
| Strain | (h) | Glucosc | Ribitol | Xylitol | Erythritol | Glycerol | Ethanol |
| 1-1 | 48 | 157.40 | 0.21 | 0.00 | 27.95 | 22.26 | 21.35 |
| 1-1 | 48 | 157.21 | 0.19 | 0.00 | 29.15 | 22.48 | 22.17 |
| 1-16a | 48 | 157.43 | 0.41 | 0.00 | 34.98 | 19.00 | 20.21 |
| 1-16b | 48 | 153.93 | 1.16 | 0.04 | 38.26 | 19.18 | 19.84 |
| 1-16c | 48 | 151.54 | 1.30 | 0.00 | 40.72 | 20.22 | 19.62 |
| 1-16d | 48 | 157.13 | 0.45 | 0.00 | 36.19 | 19.25 | 20.49 |
| 1-16e | 48 | 150.44 | 0.55 | 0.04 | 37.52 | 20.16 | 22.00 |
| 1-1 | 100 | 29.33 | 0.81 | 0.05 | 83.56 | 28.66 | 19.88 |
| 1-1 | 100 | 27.02 | 0.78 | 0.10 | 84.16 | 28.40 | 20.62 |
| 1-16a | 100 | 25.52 | 1.30 | 0.11 | 102.91 | 22.81 | 15.88 |
| 1-16b | 100 | 0.10 | 3.21 | 0.00 | 123.56 | 17.62 | 19.74 |
| 1-16c | 100 | 0.05 | 3.72 | 0.00 | 127.55 | 20.16 | 13.29 |
| 1-16d | 100 | 5.09 | 1.54 | 0.00 | 114.16 | 22.23 | 18.81 |
| 1-16e | 100 | 1.11 | 1.47 | 0.00 | 105.63 | 23.33 | 23.37 |

TABLE 14

| Strain | Yield (%) |
|---|---|
| 1-1 | 30.9% |
| 1-1 | 30.8% |
| 1-16a | 37.5% |
| 1-16b | 41.2% |
| 1-16c | 42.5% |
| 1-16d | 38.7% |
| 1-16e | 35.3% |

What is claimed is:

1. A genetically engineered yeast cell, the engineered yeast cell comprising:
a deletion or disruption of a native gene encoding an erythrose reductase enzyme at least 60%, at least 65%, at least 70%, at least 75%, at least 80%, at least 85%, at least 90%, at least 95%, at least 98%, at least 99%, or 100% identical to at least one of SEQ ID NOs: 63 and 66.

2. The yeast cell of claim 1, wherein the yeast cell is an osmotolerant yeast cell.

3. The yeast cell of claim 1 or claim 2, wherein the yeast cell is a cell of the subphylum Ustilaginomycotina.

4. The yeast cell of any preceding claim, wherein the yeast cell is selected from the group consisting of Trichosporonoides megachiliensis, Trychosporonoides oedocephalis, Trychosporonoides nigrescens, Pseudozyma tsukubaensis, Trigonopsis variabilis, Moniliella, Ustilaginomycetes, Trichosporon, Yarrowia lipolytica, Saccharomyces cerevisiae, Penicillium, Torula, Pichia, Candida, Candida magnoliae, and Aureobasidium.

5. The yeast cell of any preceding claim, wherein the yeast cell is a Moniliella pollinis cell and the gene encoding the erythrose reductase enzyme is at least 60%, at least 65%, at least 70%, at least 75%, at least 80%, at least 85%, at least 90%, at least 95%, at least 98%, at least 99%, or 100% identical to at least one of SEQ ID NOs: 61 and 64.

6. The yeast cell of claim 5, wherein the cell has a deletion of at least one allele of the gene encoding the erythrose reductase enzyme.

7. The yeast of claim 5 or 6, wherein the cell has a deletion of both alleles of the gene encoding the erythrose reductase enzyme.

8. The yeast cell of any preceding claim, wherein, when the engineered cell is used in a fermentation process in the presence of dextrose, titer and/or yield of erythritol production is decreased relative to titer and/or yield of erythritol in an equivalent fermentation process using an equivalent cell in which there has been no disruption or deletion of a native gene encoding an erythrose reductase at least 60%, at least 65%, at least 70%, at least 75%, at least 80%, at least 85%, at least 90%, at least 95%, at least 98%, at least 99%, or 100% identical to at least one of SEQ ID NOs: 63 and 66.

9. The yeast cell of any preceding claim, wherein at least one copy of the gene encoding the erythrose reductase enzyme has been replaced with a native or exogenous polynucleotide sequence.

10. The yeast cell of claim 9, wherein the native or exogenous polynucleotide sequence is operably linked to a heterologous or artificial promoter.

11. The yeast cell of claim 9 or 10, wherein the promoter is a constitutive promoter.

12. The yeast cell of any one of claims 9-11, wherein the heterologous or artificial promoter is selected from the group consisting of pyruvate kinase 1 promoter (PYK1p; SEQ ID NO:4), 6-phosphogluconate dehydrogenase promoter (6PGDp; SEQ ID NO:6), glyceraldehyde-3-phosphate dehydrogenase promoter (TDH3p; SEQ ID NO:8), translational elongation factor 1 promoter (TEFp; SEQ ID NO:9), modified TEFp (SEQ ID NO:7), phosphoglucomutase 1 promoter (PGM1p; SEQ ID NO:10), 3-phosphoglycerate kinase promoter (PGK1p; SEQ ID NO: 11), enolase promoter (ENO1p; SEQ ID NO:12), asparagine synthetase promoter (ASNSp; SEQ ID NO:13), 50S ribosomal protein L1 promoter (RPLAp; SEQ ID NO:14), and RPL16B (SEQ ID NO:15).

13. A genetically engineered Moniliella pollinis cell comprising a deletion or disruption a native gene encoding an erythrose reductase enzyme at least 60%, at least 65%, at least 70%, at least 75%, at least 80%, at least 85%, at least 90%, at least 95%, at least 98%, at least 99%, or 100% identical to at least one of SEQ ID NOs: 63 and 66.

14. The engineered M. pollinis cell of claim 13, wherein the native gene is at least 60%, at least 65%, at least 70%, at least 75%, at least 80%, at least 85%, at least 90%, at least 95%, at least 98%, at least 99%, or 100% identical to at least one of SEQ ID NOs: 61 and 64.

15. The M. pollinis cell of claim 13 or 14, wherein the native gene is at least 60%, at least 65%, at least 70%, at least 75%, at least 80%, at least 85%, at least 90%, at least 95%, at least 98%, at least 99%, or 100% identical to SEQ ID NO:61 and the ER enzyme is at least 60%, at least 65%, at least 70%, at least 75%, at least 80%, at least 85%, at least 90%, at least 95%, at least 98%, at least 99%, or 100% identical to SEQ ID NO:63.

16. The M. pollinis cell of claim 15, wherein the native gene is at least 80% identical to SEQ ID NO: 61 and the ER enzyme is at least 80% identical to SEQ ID NOs: 63.

17. The M. pollinis cell of claim 15, wherein the native gene is at least 90% identical to SEQ ID NOs61 and the ER enzyme is at least 85% identical to SEQ ID NOs: 63.

18. The M. pollinis cell of claim 15, wherein the native gene is at least 95% identical SEQ ID NO: 61 and the ER enzyme is at least 90% identical to SEQ ID NOs: 63.

19. The M. pollinis cell of claim 13 or 14, wherein the native gene is at least 60%, at least 65%, at least 70%, at least 75%, at least 80%, at least 85%, at least 90%, at least 95%, at least 98%, at least 99%, or 100% identical to at least one of SEQ ID NOs: 64 and the ER enzyme is at least 60%, at least 65%, at least 70%, at least 75%, at least 80%, at least 85%, at least 90%, at least 95%, at least 98%, at least 99%, or 100% identical to at least one of SEQ ID NOs: 66.

20. The M. pollinis cell of claim 19, wherein the native gene is at least 80% identical to SEQ ID NO: 64 and the ER enzyme is at least 80% identical to SEQ ID NO:66.

21. The M. pollinis cell of claim 19, wherein the native gene is at least 90% identical to SEQ ID NO: 64 and the ER enzyme is at least 85% identical to SEQ ID NO:66.

22. The M. pollinis cell of claim 19, wherein the native gene is at least 95% identical to SEQ ID NO: 64 and the ER enzyme is at least 90% identical to SEQ ID NO:66.

23. The M. pollinis cell of any one of claims 13-22, wherein both alleles of the native gene are deleted.

24. The M. pollinis cell of any one of claims 13-18 and 23, wherein both alleles of the native gene at least 60%, at least 65%, at least 70%, at least 75%, at least 80%, at least 85%, at least 90%, at least 95%, at least 98%, at least 99%, or 100% identical to SEQ ID NO:61 are deleted.

25. The M. pollinis cell of any one of claims 13, 14, and 19-23, wherein both alleles of the native gene at least 60%, at least 65%, at least 70%, at least 75%, at least 80%, at least 85%, at least 90%, at least 95%, at least 98%, at least 99%, or 100% identical to SEQ ID NO:64 are deleted

26. A method of fermentation comprising
contacting a substrate comprising dextrose with the engineered yeast cell of any preceding claim, wherein fermentation of the substrate by the engineered cell produces less erythritol than fermentation using an equivalent yeast cell that does not have a deletion or disruption of said ER encoding gene.

27. The method of claim 26, wherein the fermentation temperature is at or between 25° C. to 45° C., 30° C. to 40° C., or 32° C. to 37° C. and the volumetric oxygen uptake rate (OUR) is between 5-80, 10-75, 15-70, 20-60, 30-50, or 40-50 mmol O₂/(L·h).

28. The method of claim 26 or 27, wherein rate, titer, and/or yield of erythritol production is decreased relative to an equivalent fermentation run with an equivalent yeast cell in which the native gene encoding the erythrose reductase enzyme has not been deleted or disrupted.

29. The method of any one of claims 26-28, wherein the concentration of dextrose is at least 100 g/L.

30. The method of any one of claims 26-29, wherein the erythritol titer is less than 20, 18, 15, 12, 10, 5, 1, 0.5 g/L when the fermentation is run at 35° C. for 96 hours; wherein the erythritol production rate is less than 0.5, 0.4, 0.3, 0.25, 0.2, 0.15, or 0.1 g L⁻¹ h⁻¹; and/or wherein the erythritol yield is less than 35%, 30%, 25%, 20%, 15%, 10%, 5%, or 1%.

31. The method of any one of claims 26-30, wherein the fermentation process does not produce erythritol.

32. Use of the engineered yeast of any one of claims 1-25 in a fermentation process.


SEQUENCE LISTING (81 sequences)

SEQ ID NO: 1 - protein, 349 aa, Lactobacillus rhamnosus
MKASMLEDLNKFSVKEIDIPSPKKDEVVVKVMAAGTCGSDSHKMISGWKYGYPAVMGHEFSGIVTQLGENVSNVSVGQHVAVAPFIPCFKCHYCQIGLFQMCENYSMLGQQKFGGFEQYVSVPARNVLDIGKMSFEEGALIEPMAVAAHAVMGIKPELGDTVAVFGLGTVGDLVVRLLISSGATNVIGIDIDDQKLEKGLDEGCTHVINSAKESLEEKIMEYTDGLGVDISMECAGSKITEEQTLLVTKRRGKVGFVGIAYSDVLLHQKAFENIFRHELTVTGFWNSYSAPFPGREWTNSIQLVNRGRIKIKDLITHRFELEDMQKAFNMITTRSESFNKVMFFPNGIN

SEQ ID NO: 2 - DNA, 4576 bp, synthetic construct
  source 1..4576: mol_type = other DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcggtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaagcttccatgctggaggacttgaacaaattcagcgtcaaagagatcgacatcccgtcccccaaaaaggatgaggtcgtcgtgaaggtgatggcggcaggcacgtgtggatccgattcgcataaaatgattagcggatggaaatatggctatcccgcggtgatgggacacgaattttctggaattgtgacacaactgggagaaaacgtgtccaacgtcagcgtcggtcaacacgtcgccgtggctcccttcattccctgcttcaagtgtcattattgtcaaatcggactcttccagatgtgtgaaaattattccatgctcggtcagcaaaaatttggaggtttcgagcaatacgtttccgtcccggcacgtaacgttctggacatcggcaagatgtcgttcgaggaaggtgcccttattgaaccgatggcagttgcggcccacgctgttatgggaattaaaccggagctgggtgatacagttgcggtcttcggcctcggtacagtcggagatcttgttgtccgtcttcttatttcctcgggagcgacaaacgtcattggcattgacattgacgaccagaagcttgaaaagggactggatgagggttgcacacacgtgatcaactctgccaaagagtctcttgaagaaaagatcatggaatacaccgacggtctgggtgtcgatatttccatggaatgcgccggcagcaaaatcactgaggagcaaacactgctcgttacgaaacgccgtggaaaggttggatttgtcggtattgcttatagcgacgttctgctccatcagaaagcatttgagaacatcttccgccacgagcttacggtgactggattttggaattcctattccgcaccatttcctggacgcgagtggaccaactcgatccaattggttaatcgtggtcgtatcaagatcaaagatctcattactcatcgatttgagcttgaagacatgcaaaaagcattcaatatgatcaccactcgttcggaatcgtttaataaggtcatgtttttccccaacggcatcaattaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 3 - DNA, 500 bp, Moniliella pollinis
  source 1..500: mol_type = genomic DNA
tgcagagccttgcaaatgatgaaagaccaatggaattgatcattgattattgatgatgcatgatgaaagagatagaaacagatgcaagatgggatggcgcgatgatcaaatgataatgaacgttcaacccttcaaaattgaaactctcaggctgagcggctgagcaagctctgagaatgtgggtcttcgaagactttcgcttgagcatggtgcatgtgcaaagagatggaatgtggaattgttgtaggtaggaacacagcccggtagagcggcgcagtggctcagcgaagctgggagaacgactgaaccggtcttgagattcatcatctcagcgtgcgtcagcgcgtcagcttcagcgtttgagaatggcacggactgacctgtcgtacatcccaaggaggcccgtgcgctatactcgccttgtggcctaatctgcgatcaacccatcctcaacgaaagcgcaagtagcgactaagcacactatctttcgggtcggtgcg

SEQ ID NO: 4 - DNA, 1002 bp, Moniliella pollinis
  source 1..1002: mol_type = other DNA
tttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaa

SEQ ID NO: 5 - DNA, 1050 bp, Lactobacillus rhamnosus
  source 1..1050: mol_type = other DNA
atgaaagcttccatgctggaggacttgaacaaattcagcgtcaaagagatcgacatcccgtcccccaaaaaggatgaggtcgtcgtgaaggtgatggcggcaggcacgtgtggatccgattcgcataaaatgattagcggatggaaatatggctatcccgcggtgatgggacacgaattttctggaattgtgacacaactgggagaaaacgtgtccaacgtcagcgtcggtcaacacgtcgccgtggctcccttcattccctgcttcaagtgtcattattgtcaaatcggactcttccagatgtgtgaaaattattccatgctcggtcagcaaaaatttggaggtttcgagcaatacgtttccgtcccggcacgtaacgttctggacatcggcaagatgtcgttcgaggaaggtgcccttattgaaccgatggcagttgcggcccacgctgttatgggaattaaaccggagctgggtgatacagttgcggtcttcggcctcggtacagtcggagatcttgttgtccgtcttcttatttcctcgggagcgacaaacgtcattggcattgacattgacgaccagaagcttgaaaagggactggatgagggttgcacacacgtgatcaactctgccaaagagtctcttgaagaaaagatcatggaatacaccgacggtctgggtgtcgatatttccatggaatgcgccggcagcaaaatcactgaggagcaaacactgctcgttacgaaacgccgtggaaaggttggatttgtcggtattgcttatagcgacgttctgctccatcagaaagcatttgagaacatcttccgccacgagcttacggtgactggattttggaattcctattccgcaccatttcctggacgcgagtggaccaactcgatccaattggttaatcgtggtcgtatcaagatcaaagatctcattactcatcgatttgagcttgaagacatgcaaaaagcattcaatatgatcaccactcgttcggaatcgtttaataaggtcatgtttttccccaacggcatcaattaa

SEQ ID NO: 6 - DNA, 1002 bp, Moniliella pollinis
  source 1..1002: mol_type = other DNA
aacacgtagaacacagggatcgccatttgtccgaaatgcaatctttgctctcgaagcttgaagcttgaatagcttgaagcatgagtgcatgaggggctgctatgatcttccaaccaaacttgcttgggcattgcactttggctggatagctatcatttagctgacccccaaagagactgccaatgatatgatatttgtgtaggtgcacgagtggatgagtggcgcgactacctgtcacaagatagatgctcgatgcacttcatgtgaagtggctagtggatgtccggagactggagtacagtaaagagtttggagttgggaacagcgcgaatgaaagaggctcagagagaggtaggtttgatgtgacgtcaagcgggctagtcagttagcaagggagtatggaatgcagcgcgcaacgcgcagcgcggagcccagtgcgcctgcgctcaggtactcatactcagtactcagtactcaggcgccatgccggagcttacatgcaatcaattaaccctgacgtctggcgcggcctgacgcctgaggcctgaggcctgacgcggccaagctccaagcgcaaccccaggctggcttgcgctggcgggcgcgagacgcagcagggggcaagtgtcgcagagtcgccgatcggatctgtggaacagcacaggaggcccgtgcggtcggctgcgcagtgcggtggagaagctggaagctggaagctggaaggtggaacctggaacttggaacttggaaaggaagaagagcgggaaggcgatgcacgatgcacgatgcacgatggggttggattgcgtacgagtagccgcacagcgcacagcgcacaacactcaccccatgcccccacgccccagcccgtgctcgggccatgagctgctggcaaccccccgaggcagccggcctttaactcaagtagcattcgccgccggtcggttcttcttagcctcccaccaccaccacttgcccgtgcttggacgctaattacaataa

SEQ ID NO: 7 - DNA, 679 bp, synthetic construct
  source 1..679: mol_type = other DNA
agctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcacc

SEQ ID NO: 8 - DNA, 1002 bp, Moniliella pollinis
  source 1..1002: mol_type = other DNA
catcgttccagcttgccttgggtgctagccagcgcaaaagtccgacgaacagacgaattgaccgtagtcgatgacagtcgatgacagtcgatgacagtcgatgacagccgacgaccgacgaacaacaaccaaccgcacgcagcatgaggccgagaggctcaggctgagccgctaaggcgctgaagcttgaagctcggtggtccagtcagatctcatgatctcacaccgccgcgctgctgcggtcgtatggttcaatttgctgtgcctagcctagcctgggtacgtcggtaactagccactagccactggccattatttggctactgggcacactaggaggcgcactagtccatcgcaagcggtaacttacctaccggccactacctgatgatttccctcttgcctggcggcctggcggcctggcggcgcactctttgcgctcggacccgcaaattgcaaaccaacaaacgactagtgtccggtacgtgtgcccaagtaacctgggcagacatagaaccacattgatcgtagggaaccgcaatggaccagtacgctattctcagtacattctgtacacttttgcctagattggaatctggtgaggatcaagatcaaatttcaaaccaactccacggcactggttcccgacacttggactgtagtactgaggtactgaggtactgaggtactgaggctcaactgagtctcaactgaccgtgaccgcgcttgtgtggtggcgtggattctagtgttggatgtgcgcagtggtgtgtgcggtggtgcgttcagattcgtcggtagaacttgactattcacgtcttgtgctgcgctgacgcttggaacctggaatgttaaaccttgggtcctgagggtctccaccccccaagtcgactgggttctatataaagtcgggtaggcaattggctagggagaatctctcttttctctttccccagaacacctgttcactcacttctcacctctttacttacctacttcaca

SEQ ID NO: 9 - DNA, 1000 bp, Moniliella pollinis
  source 1..1000: mol_type = other DNA
cctcgttcttgattgctttcaaaagtaatttaaggagagaacctggtcgtttgttgctctactgaatgatcttacctataccagcatacgacaccttgctgcccctccccttcaccaccaccatcaccactttcctctttttctttgggtttttctagtcgttgcatttatggttgattcgctctttgagaaccgtatggctgttattccttcccaactcttttgtctgttttatcgttgcatccaaacgtttgggctcttcgtacctcttttttctctgtttgccttttatttttggtttttctatccgtttgatggttgtgactcttgttttcctttgggaaggtttgagctcaacttctcttcggtccttgttggagtttgacttccttttctttttcagctgtggccgatcattacgatcaataatttgtgaatatgatatcgactacatcctgaatcttttagcgaaccatagcagtttagcgtcatagatatgcatgcatgatgtacatcttacacgcaccacggatgatgctcagcgctcagccacgcagccgctcagccgctcgctcggtacgctcggtcatgatgtgcatgatgcaagcctaatgattgacgggtacttgatctgtgtcccaattcgatcgagatcgatctcactatagtttgtcgaacttcgatgcacatcgagggtgcggaaaaaagctgacggcagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaaccctgccggccctatataactagtgcttcatcttcttgtcattcttgtctctttaccaacacaccggtactctttcacc

SEQ ID NO: 10 - DNA, 1007 bp, Moniliella pollinis
  source 1..1007: mol_type = other DNA
cttgacgggttcaatattcaatactcagtgttcaagcagtggtaggttgctccgtggatggaacgacgggtttaatttcttccgccatgcaccatggtccatgatcggttcagatggacagttggtatgagaaagtggtaacagataaatagaatttcgtacaagttggcaagagcctggccgatggtccatggctcgatggctcgatggctcgatggctcgatggccacacaccatgtcatctactttctgtgcttctatgcggacgagtcggaacagctgtcaaactgcgtttggggtatttgagcggaatttgaaaccttgttgaaactacattgtctcctcgttacttctctggcatctgtttgatcgaacggcgtcatgcaatagacacgcgatgaggcccaacgacggtgacgttgaacgcctggcccacctgctacaacaccgtagaacgtgttgaagttgaacgtcggacgttggacgttgagttgacgcgtgatgctggaaccgtggatgtagcagcgacagacaagccctctaagcaatctagtcgattttcaaaccaactaaacgagccttagttcggacaaccctagtccagtgcgggggcagcgtggggtcagttgccagcgaaggtcaccccagctagctgggttggctagcttgactggctcagaagtatcgatgctggagcttcaggccagtacgaaaatacactgtcgtagtgctccttccggagttctgggcttctgagcccggcggttgtgcatcggtgcacagtgcacggaccggcgggtgggacaacaaactgtctgggcgtgaaatcgtgcgagtgttgatgcaaaccggacgtacccagcacgcagttcccagtactcagacatactcagtacctcagtggttgagcctcagcatcgcctccgtatttctacaaagctgttgagtcgccttttgactttttactaccaccatcccctcttcttctaaaccccttacattccaaa

SEQ ID NO: 11 - DNA, 1000 bp, Moniliella pollinis
  source 1..1000: mol_type = other DNA
tcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaaca

SEQ ID NO: 12 - DNA, 997 bp, Moniliella pollinis
  source 1..997: mol_type = other DNA
tcggaccccaagctctctagcttcaagctttcaagctagcttccagccagggtcatggaaccgtaggagctgggaaacgtgacgttgtcgctatgcctatcccatataagaattaacgtcgaatcatgaggcgcatgggaggctgcattggtggtatgatacataacgttatgattctatatgagcgatggagcgcggccgtctgccgctagtgtggaagttcgatacgcccagtccatttcgttcagatgacgtttggtccagaagtacggtacataccgagcgcaaacgtcgaacttatggccttggtcgctcgtggtccggcgtcgtcgacactttcatcaacggacgaaaggtagagccgaacgccagcggccaatggcgatggcgatagtagaatgaatagtcgtcagatctaggtcttaggctacaggccaccctgcctgaggcctgggggtcttacagcgagtctaaaaaacgaaaagccaacgtgccaacttgggcctgcccatgcaccctgccagagagccccaagctggtctgatgcaagcggagcaaaatgttggaagaatagtttcaacgatggtttggttccagtacgcacagtcgagagatgaggtactgcgcaacgagttgttggcgacggaaccgaagatggattgaactatttgatctacaggtgatggcgtggagtcgggcgtggggccgctgtgtgatatgtagaagtggcgctattcgagggctgcgcccgtctggatggatgcagaatgaagcttgcaggccgcagctggcaggatgggcgcagaacccggtgaaacatagttaaagcgcggggccagtgtcatatttcgcttgagccattctactcgttggcagcccaggcgccgctaatttacgccaactcttggtactcgagagctcgggcaatgtggtatataagcttgaggttccctacagcaaagcctggcttcttcaccacaaccgcactctagcaaac

SEQ ID NO: 13 - DNA, 1002 bp, Moniliella pollinis
  source 1..1002: mol_type = other DNA
gagcgtagagagctccatcgatggcctggttacaagttgcctgaagaccaccaatacggcccgcacgccaacggcaggccaagaaaggataattgacaaccgtgaatctgaaagcttcggcgctcaggagctcatgtgttttgttgtagatataaaggtttctgaataccagttgggtattgacacggggagttggactgaatctctcagatctcccagatctcaagtactgagtcccaagcgtgccataattaactctaggttgcgcgggacacgccacctgagatcagggtgtacgcgcatactaatccattccaccaccaaagcaagactgagggtaccgcctgaggttctggctattccaacagtcacaagtcaccgcgattgccgtcaaattccaaagcgtagtaggcgctcgacggagggatgatggccagtacccaatttcgagcactcagttagatctgttcgtgtttagttgctcctgcagatctatcttaaatgtatactgtgccaggtaggatctctcggcacctatagatgatataaaagatatgatgatatgccaagtgccaagccgtcaaactgacaaaatgaaagaaacagcaggcaagctaggcaagcgtctatcgggggattaaacggatgagtcagcagcacaggagcagtcagtcgacaattccggctttttcccgcccgcaaggccccagaccccagaccccagccgccaccatgtccgtatccgccggggttagcatgaccacggcagatttctccgctcagcttgctcggacgactatatataaagaatcctttcctcgcagtccgtccgagcccttcctccaccccatcggttaccaccaacgacatcacttggtcttgacgccgcgctgcgttttcccgcttccgcgtctccctagtgatttagctggtgttttcctctacccgccactctactgtttcatcgcttgttgccaagttttctcagttcattctcatcaa

SEQ ID NO: 14 - DNA, 1002 bp, Moniliella pollinis
  source 1..1002: mol_type = other DNA
tggacgtggacaagcaccctgaagtcgctcagggcttccagatcaaggctttgcctaccgtctacggcttgtatgagggcaagcctgttcgtcagtgtaagttttaaaatatattgttcttgtctgttcgggttttacatcagtggtgtactgacacggctttttgttttattgctgcagtccaaggtgtacttcctgaggatatcctgcgccaatttgtccaagatcttcccagcttggccgcccaagatgcccagaacccgcctgctgaaagtagcgaggctgccaactaagtatagtccatcctatgagctgtcctaccccaaacaactttttcacgcccaacgtccaaacgtcacttcttattgcggcaaagaacagctgcttccccactcattcattacccctttctgatccacgaggtgtacccatggcctttgtacattccatgaaacaggtatccttttccccaccattacatctgaacttacaaagactttgcttcctcttgtacacagctcgtcgtccatcgtccatcatcgatgacgatcatcctgcattcggtatcatgcaccatcatgcagcgatggcaacgagggccatgcaccatgctgcatatactatgtgtatagccaatagccaatcttcaatctccaacttcctacagacacagcctgtggggaatcgcttaatcgctgtccatgcgtccatgctccgcgttcctcgcgctcgtcgcggcctcgcgacgattaagtcacagtcacgcccacacagcgcgcgagcgcacgagccaccatcatcacgtgatgagacaaagagtccacttccgtaaaagcaatcttaataatacatgtaaccctagcaatgtagccctaataatttgtttggcctttattttattgcgcccccgcagcttttgcaccccggagagtaaaagtattccggttgctcgtcagagcatccctttctttctccatcagcaaagcgtgcgaccgccaagaa

SEQ ID NO: 15 - DNA, 921 bp, synthetic construct
  source 1..921: mol_type = other DNA
tttggatgaggcattcaatgtctccatgtttgaagaccagagaaagtgtaggtaacacaatagccatggacaattagcgaatccgaatatgtgtactaacattgtgcctccaactgacagctggtcgaataatgattgaaatcatcaaacatatcagagatatatcccagaaccatttaacaactgttattgtatgtatgcattactgttttacattgtgtctttccaaatactaacattgccattatccaatccatacatatatatatatatagattacacaacgcaaacctcgacagtcgattggtttacatcgtgtaagcagccttttacggaatatgatggacaaccagatcctcatagatggagacaatgtgcagatagattccatttatgatgacattgatgtgtttttaaatgcaaaataatctcctatataagtgagttccgagtctccccatgcttatcttatcgtgttttttctgtaaattaggtcaaagtattcaaacaattccctcaatctttcccattttttgctgagcccccatatcatcaaattaacgtatcaaatttcctaattagtagccccgtatgttaaattagacatgtgtgactgccatcccggacagcctacccaatgacacccgcgcaccgcacatattgtgtttagtgcgcgccgtctgctgaagcgactccctgtttgggaggaaccgagggcgggttgcccgatcccttgcccctcgctcctcctcctgggctcccccttgcagagggacaccgaggggatccctcgtgtgagagcttggaggtggatggtggtcaattttctcatttgattgaaagatttgttatattgaaaattcagtttgtggaagttgtgattaaaaggttttactgtttgttctgtagacacattcaata

SEQ ID NO: 16 - DNA, 500 bp, Moniliella pollinis
  source 1..500: mol_type = other DNA
acggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcct

SEQ ID NO: 17 - DNA, 500 bp, Moniliella pollinis
  source 1..500: mol_type = other DNA
gtactaaccaagtctattgtaaagctggtgtctgctgctgcttcagcaagtgccattgctaccatcgcaagcaaattgcaaaaaaattcgttcaatggtctaatttaagtaaaatactcataatttataaaatgccgcttgtccttaccttttgtctttctccttattatagtagctgtagttatcgtagtcctcaggacttcgtcaacgctcgttgatgatcatatatacttcacctcattcattcataaagcattcattaggattgtatgatccggcagtgcatcatgcatcatgcatcatgcatcaggacatcatgcatcatgccgtatcggtcgatcattgcatttgattggataatacgatcgtgtttaggcattggcagtcgaaacgaggataaagaagaggtcagtaccagtgcacagcacgcgctcgcgctctatgcgcactttgacgcttgaaaaaaacttttcagccaatcggatatagacagcggaccc

SEQ ID NO: 18 - DNA, 493 bp, Moniliella pollinis
  source 1..493: mol_type = other DNA
atggtctgagccgcccacgattccttccttcatgagcgtatgacgacccttttacttagcatgtgcctctaaagaatacgttttgcatacccacatattcgtaaccctgtcctctatagcttctacaggtcatagtcactggctgccagtataaacgtgtcaatatctaatcctcataagatgcctcgctctgtcgtccacaataaaattcagtaacgcaaggactgcgcgactgagtgattgccgatgatatgagaaaactaatttcaagccatttgaacttcaataaatgcttccgctaacatcacggatcatcgatcatcaaccagcctccattaataggcctgccaccactcgcgcaatcatgaatctgatcatacctggctagcgattctcataacaccagaattacctgcccattaaagaaaccctattctgcctgtaacccggcaccagccggtccgaattcacgacagactaaggctctcgcgca

SEQ ID NO: 19 - DNA, 500 bp, Moniliella pollinis
  source 1..500: mol_type = other DNA
atcagttggaatcatcaaccttttttttttgtatcgatttcttcctaccctcacctcccttttcccccttaactcctcgctttgaacctccttacccgagtgcctacctttacctttttctttttctctctcccctgcctaatattctaggcttgctactatactgctctctctaacacgatttttgcgcttgtaagcatttttctctttttaaatctaggttttatcatttgttgcatttatccaagtttttttttttttttgtttgtagtggttttactgtttttgtgttttgtagcttgactaggttcttattctattgtagatgtatatgatcggatgcattcccgatctaaaaggaaaatgttctcgactgatatttatttttaccttattgtcttcctaaatggtttgactcttgccttcttccttgggacgctttgcaacgttttgtacgtacgtaagggttcatcgtttggataagcgacacagttgtcaag

SEQ ID NO: 20 - DNA, 324 bp, Moniliella pollinis
  source 1..324: mol_type = other DNA
attcaccctggagtaactgcgcttcatcgtttctttgttacgacaataataccacgctgcatgctttgaattgagtatatattttgatgactggttcaaatagatctttcctccatagccttcgagtactcgtgatctcagcattatcagatttgtgtccaggcccccgaggacgatgcggttttcctggagtcactagttgggaagatttgtactggttgggtgtaggtagagagaaccattgattttggtgtagctgttcgttttataaggccgagattacgtaatgcacatgctgccctagcaaatcgcgtcgcgtcacgc

SEQ ID NO: 21 - DNA, 496 bp, Moniliella pollinis
  source 1..496: mol_type = other DNA
atgtgcttagattaacatacacttgacttccttacgcgttgatatatacttggatctccataacttctattgtatagtttaatcaatggactccatgttgacatcccattatgcgatctatagtagctggaaatagcaaaattgaagttcggcagacgactgcaccaggaggggtagattctgtcccataactccctaccacaggccgtgctctggttcgatttaccttagcctgatgcgctcaatgtaacgcgtaattctgagtggggttcggtcgtccaaggtttcagtatgcgctgtgcagatagtactgtatcgtgaaactcgaccgttgcacatgcactttatcaagtgtcgtaggcccagagtcgtaggccaagagtcgtaggccaagatggaaggcctgaagaaaaagaaagctttgatcgttctgatctatgcaatggattcatgagctaagtattagctatacaaatgattcgagcatagcagaagg

SEQ ID NO: 22 - DNA, 500 bp, Moniliella pollinis
  source 1..500: mol_type = other DNA
gttgctccctttgctgttctatgatccaaaagatgcttagtgctccactctctactcccgactaccttttacgcattctgacatgactagtagtgcaatgttacccctggtgttttctcgcaatcactttctgtctgctatagcttttgatcgtcagtagttttttggtatagctaaagtagacttttgtacctggcgttcaatgtaattttactccttttttgtgcgatgcaacagatggtgtaaagatcgtcgttaaagttttggcttgatgcttgattcggtgacggttgcttgggtgatccttatatcgtctcctggtgttctcataagcccaggctcttcttagctatgtacaacaggtccaccactgactatgttatgtagaggcatgcatttaacttacggaaagatttaatggagaagaacgtacctagtcagatgcttgacatgaaaacgtattcctgcaattcacaaatcagtatcctgcctgtccactc

SEQ ID NO: 23 - DNA, 500 bp, Moniliella pollinis
  source 1..500: mol_type = other DNA
gcggttcgcacttttatcccaggcatgaaaaatgacgcacggctgtcgcacgctacacccagtagtcgccttttcaggcagtctatgaggggagcttttcattgcccaaaattactagcctgcaattcatgatatcaggttcactttctcccgatgtctatagatttctcatagcgctttgaaaagacaatttagctactcagtttataagcaaatgatgtggaggtatttattaggtacaagatacgataacgtttacacgcggggaagcaagtggcaaatcgtcgacaacaccgcatggtggtggtggaatcgtactccatcaaagcttgcttgtcactttgggaggcaccaggttgaggccatcgcccgaaggtggttgtggccagggtttcgatatttcatgaatcaacgtgttgatggtattggattgggtaagaggtcgatccggctgttggtcagagtgctcatcacattcgtcgttgtcaagcccgagttgc

SEQ ID NO: 24 - DNA, 500 bp, Moniliella pollinis
  source 1..500: mol_type = other DNA
gtggcgttgattcgtatcttattattgatctgtcctgttccccacgttgccaagtgattgttcatgctatcctctctccattctgtttgtgttgcacagtcggtagttgtacgtagtagctcgtcacaaaatatgaagtgagccttgcaatgtaggcattcacaagctgctaggcaggctatgatctaagctaagtggcaaaaattctcttgattcaatgccaaactatactattgttcctcaggcaaaagcagctatataagttgcattcaacgcaaccgccatacaagcaaattagagagaaacgggatataaggtgctttatccctgaaggagtagccagcgtctacttggttgctacacaatacagcaaacagtgtacttaatgagccattggctgaactggagctcgcagaggttcgagaagggcgggtcataccagctgccctccatggaagctggggtgtcgatgatgaaaagtgtcggaacgtacgctagta

SEQ ID NO: 25 - DNA, 500 bp, Moniliella pollinis
  source 1..500: mol_type = other DNA
acgaagggtttcccatctgtgtgacctgatccctttttacgtttttttgttttgttttttcttttgttcatgatcaacttgggagtgattcctcttggtgatgtcgaaaattgaactgttataccaatattctgttgcctttgtttcctgtttgtagtttactactttttacagtagtcgttgttcctgccttgcttgcgtttgtcagggtttccctttagcttagtaccaatatctagccttaattttgcttattctttctcaccgcccatgattgatttcaatcatgattaatgatttatgcatgaaaactcaaaatactcaattgtcttgtacaaggcaatcacaagctggtcagccattctcagcgcctataagagctttcagccaggccggccccaaaacgatctaccctgtggacctggctaggctaaactttggaaagtcatgcaattatgaacgtttgaagctcaagcgctcctaccctctcctggagcagt

SEQ ID NO: 26 - DNA, 500 bp, Moniliella pollinis
  source 1..500: mol_type = other DNA
tggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgata

SEQ ID NO: 27 - DNA, 500 bp, Moniliella pollinis
  source 1..500: mol_type = other DNA
gtttcttggctgttgacgaaatcttatgaaatgcacgggtctaatatttcctgccaattgtgtacccatctcgtttgtgaatgtcatagctagctctgtagtttctaaacgaccgtcttaggccggggtttgctgattggggattaaggataggatacttggccagtttgggccgatcaatatcaatctgctaggcgttccttaggtcaacttgaattatgatatatgaggtatgaggaaagtgggctaaatctgtggattaggatcagaaaaaacgtggcagaccctcgatctaggcctacgtcagcaatttcagcaatttcagcgccttgccccttagataggtttcagccgctcagcgcgcgcaatttcagcatggatgagatcaaaccaaggggcagcagttggcgaccctgtagttccaatcgcatgcttagtgcataacgcgcacatcaacgtgtcagattctcagagatctcaacaagcccactagaccctag

SEQ ID NO: 28 - DNA, 504 bp, Moniliella pollinis
  source 1..504: mol_type = genomic DNA
aatcgcctgtgtagcgaacggggccagctgggtaaggccagcggccggtagctaggactatgaggctaccaggctgctgggtaactaggcagctaggcacctaggcaactgagcaactgagcaactgggcaactgagcggtataggtttggcttgggcacagttgatattgatatcaaccagtcgctcgttcggttcaccagaacccagtgtgtggtagcgagcgtagaatagccaaaaggttttgagctgaggcgctgaggcgccacgggaggacatcaccgcctgagcacacaggatttcaaactgtcagacgggcgtggtgccagtgttggaagtgggcgaagcacctcgcatctaagacatgtaattaaactctcgacagccatcgtccattgacaccaacgtgcaccaacacacgcgagagctgcttcgttgtgcctgtcgacgacattgcctcgttccccagccaagtcgccccaccgctacaccaagcgcactcgca

SEQ ID NO: 29 - DNA, 499 bp, Moniliella pollinis
  source 1..499: mol_type = genomic DNA
catccgaaattggtggtttgacaaatgcaccttcttctccaggggctcgtgcaaacgcagtagccgagaagaggcgtaccggagaggaagacccaacaaccactgatcggtttgaagttggatgagaaagttgtgccaatcgcggagccgtagcaacgctacgagcgctacctacaacgactccacgcgcaatcggtacgcgcaaaggaacggatgagcgaccgagcaggttgcgcgaagccatgacgattgctctacccccaacgagctggatcaagttgggcagaacataatttatctcgcttccccgcgccacaagtagctccaccattcttctcaaaatagcccgattccgctttcgcggggcaaaacttccgcctataccgatccgattgaaacgccaaaaaaattaagttgtagtacgcctgttggtcgagcccgactggagcaccatttgagacactaggagataatttattcttttccttcttggccccca

SEQ ID NO: 30 - skipped

SEQ ID NO: 31 - DNA, 453 bp, Moniliella pollinis
  source 1..453: mol_type = genomic DNA
gcgaggagtagttactaggtagcggtatagcaattccgcccataatgcataaccgttctccctaccaaaacaaaacaatggctacgtggccggcgtgcgtggccgacatggtgcgggtgcggggggcggggtacgagtgtggggagttcactagttcgctggctagctgactagctgaccggcgtgtggttgtggcagtacgcagtgggaaagattaaatatgaaatattgaatatcaaaatgacgtttggggtggcacgtgcgcgcgccaacaacaacaagagcagccaccaagccaaagcaccaagcaccaaccccgcacaccattttgttacacccgcggcagaaatggcgtcagccagcagtcactattactcaatattcaataggggttccacttgactcacttcctcatgtatatatatatccatctaatcttcccaatacataacc

SEQ ID NO: 32 - DNA, 500 bp, Moniliella pollinis
  source 1..500: mol_type = genomic DNA
gcggcctatgcagacgcttggagcttgggcacattggatattggatattggctggctgccgttcgagatcccgttgctgtcgccaccgccaccggtcacgtggtcacttggtcacgtggaccacctgcactgcgcttagctacaagagtgcatgatgcatgatgtcatgcatgatatgcatgatacaagcatgaatattgatttcaatattgaacatttagtattgatatctcgatatgcgtaacacgtgacacgcaccatatcaatcagccgcaccggcactaaatgtttcaatttcaacatcccacacgctcgaatgctccctcgagctaatcggatttcggagctccgacatggccggcctcgagcggagtttgaaactccgttccgcccgaccccgctggtcgccgcgatgaaatcccgagtcaagtcatagcaagccaagtggctggctgactcaaaagctaataaaagtcgtagcaagtaatactccgcacgct

SEQ ID NO: 33 - DNA, 500 bp, Moniliella pollinis
  source 1..500: mol_type = genomic DNA
tcgcgtcttcaaaccgtggcgcgcgacacgtgccgcgaccaaaatttgttggtgtatgggaatagggttttctaaaattctgttgaggatgtgagtatcactggcatggcaccagtgtactgctcatcgataatcataaacgtatcaacccttcggctctgcgatctgtatctgctactggagggcacagcaagccttctgcttggttgcgctctgcgctctctgtggtgctttgccgctcgcggccaaggtccgcagcgggcattcaccaccatacgggttaagccaccagacgagtgctgctcgccgggccagctcggcgcctgtcacgcccccacattggcttgatgctcggctccttctttcatcttggaaccggcgggcaatggcgcttatcaccactcctgggccgggccccgcgtatactctcataactagtgcacgctttcaaataaatacctcgcctcctggctggccgaccgacgtttacccatccgccc

SEQ ID NO: 34 - DNA, 499 bp, Moniliella pollinis
  source 1..499: mol_type = genomic DNA
tggcatgtaggcctgtaggggctatgcctatatgctcatgcactcatgtcgcgatgttgcgatatcgcgatggaccaaccaagcaagcgtgcagcgtgggcgcttgggccaagggtgctaagctaagtgtaagcactgaagtgctgaagtgctgaagtgctgaacgtggaacgtcaggatgacacatcctctcccttcggcttcgtccaagaaaggttggtcagccatctgagatctgagatctgaaatgaatataatgatgaattgagtcgatgagatctgactatcttgggagattgcaccaatgcgccacggtgaccagatggtcttggtcttagctggtatagtggcacctgatctgccttgacagatctcatgttcatacgttcatgcattcaatcttatcgtctagccgtctgcgatctgcccaactagacgacgacaaatgtaattgatccttgtaaccctgttgctggtcgcagcagcgcgatacgatgcc

SEQ ID NO: 35 - DNA, 500 bp, Moniliella pollinis
  source 1..500: mol_type = genomic DNA
aaatcttcatccagcgtagcaagcatatcgcgcatgtccgagttcctcgagtgtgcataccgaacccaaaaagccatggatgtcattgacctgctgtattcatcaatcgagatatctttactccggagcgtacagtggcagatattatgtaggagatgatgggacaggatgcagagatacataatccatttccagttaatagggccgctagatacttgatattagcctggcagcgagcgagcgagggtagagtagaatagagtccagtatagatcccaccccatggtgcgggaagtggatgacgtaatagcgcgatagctccccgctcccttgtcacctaggccacgggaccctacgtgattgcatcgtgcgtgtcgtcgcctgtcgacgcctggtgcaggcatacgctgcgtgcttcgacgatcaacaacaccaccaaccacgactccagttccggccagccagtaccatggcgtcgttctttgggtttagtgcaccgg

SEQ ID NO: 36 - skipped

SEQ ID NO: 37 - DNA, 500 bp, Moniliella pollinis
  source 1..500: mol_type = genomic DNA
atcctgtgctcgtctcttacattcatcttcctgcccataaaactttcaagctatttcctccaacgtactcaacaacctatttttttgggccccgctcaaaacgcaccttattgcgtctaagtgtattctaaccacgcgacctgaattgcaaagcctgttacccccatcgcaccttcgtcttaagatagttgtttaacgagccttggccagttttagccttgaccatgactacagatcataatggttagatgcttcttgatcgataatgatatattgcatgtcgacgattgaagatttattggatgtcgatgacgactgtcgagctcttattcttagcaacaacttaaatattcactatcgaatgcacgcgacatcatgcatcgtgctcatgaatatcactggatcactctcgatcgcagcccactaggtcagcataacacgcttgaagctgacttgaagctttggaacaaatggcagcaacagtggcggtcacggtgacc

SEQ ID NO: 38 - DNA, 486 bp, Moniliella pollinis
  source 1..486: mol_type = genomic DNA
attgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 39 - DNA, 501 bp, Moniliella pollinis
  source 1..501: mol_type = genomic DNA
cctgaacgtcctgaactctgtcttattacataccattaatttccctgccttccctctctacgtcatcggaattttatctcttcctgagcagcaaagatttggtccttgtccgggatacaacggagagtcatgattgggtttatggaagagaggcgatagcacttgcattttgaccattcggggattaagcgaacccccaagtggcacaaagagctcgtatgagggtcagtaagtatagagggacatgcaaaggagagaaaaggaaacacaatattgacaaatagagacaagtacaggtgatcgcaatagcatgatgtaggggtatgcgcaagtcacaatcgttcagggcgggcataacatcagcgtacccgagcacaatggttaatgattaatgagcagaggaaaaaataatgattatgccggggatggcaggactaactggcgcagctcttcagcttcggcaacgcgtgcacatagtggtgtagcaccacgagattgagc

SEQ ID NO: 40 - DNA, 493 bp, Moniliella pollinis
  source 1..493: mol_type = genomic DNA
cctttgcctcacgcctctcgtttgcacgcttcttcacggcttatcaccgcttattatatccatggaatcccctccaaagttttcttcaacatagcttcatatcttctaccataacatctatcccaccaagtagcacatgaattcaacgtagcttgagaacttttcctcgtcagctgattgcgttctttaggcagtttgcttctaccccaattgcttctaccccaattgcttctgcttatttcaccagtccgctcggagatctctgacaactctgagatctgatgatcaacctcttgtgcaacttgtaaaatttgatcgatgagacgattagggacttgaagctttaagttagacgcgatgggcggtaatctatatgcttagactttgttgctatgtttgtcctataagatctgatgcatgatgtatcattacccagacgggctatcgcacaaggtgcagatgcatcttatcatgacagtgccacactgttggc

SEQ ID NO: 41 - DNA, 567 bp, Moniliella pollinis
  source 1..567: mol_type = genomic DNA
gcttgctggtcacattattgtcagtacctaccatgaagaattcaatattattttacattgtcaaccattacatggatcccatgttgtgcatagcctctatgtcatcctttggtcttttatgtctgtagcgtgctagtagtattcaagttgtagcactgctgtgaaacagtacgttcaagtagtgtgcagcagtacgttcagttagtggactggggcagcctcgccttcagcaaaggttaggtacatgagccagttgctcactcttgctttctacgaatcccataggctactttgccccctaaccgtgacgaccccctgtgtttgagaagtttgagtctttttacgttaccgtgaccaagcgagcagagtagaaactcttgaagttgtgtatgtaagaatacaaattgagaacacaaggttacctacctttatagttggtaaacagcaagcaataccagagattttcatcgcaacgtgactctatcattcactatattaagttgcacacggatgttgtacaacactacacgtaaaagagagaaaaatactaatgtcgatcgctcaggt

SEQ ID NO: 42 - DNA, 504 bp, Moniliella pollinis
  source 1..504: mol_type = genomic DNA
ttgctgctacgacctcattgatcgatgatctccgacactttgagtagctttccatcctctctttactgtactttgtattcttgctactttgtgttctttcttaatggtcccgaaccggtgattttgctcatgatctttttttttttttttcgtcaactgcaagaagatgaaaaatctactgttcgatagcacctcataaccgttccagagtcccgacaaccacaaggatgtttcttagataggtgtcagcaagatccacaaaaaagtgtaggtattagatggtttcaagcaaggtacaatattctaattcaatactcatttgaataatcaatattcaacagcttctagcctacaagcttcaatgttctccgtcggttaactcagtatacttagtactcagttcaagtcagtgttgttaaaataaaatcacgtgatccaaaaacttgaaacttttcatttttctcctcagatgttggtgattcgtcccatagcggttaccacaac

SEQ ID NO: 43 - DNA, 1679 bp, synthetic construct
  source 1..1679: mol_type = other DNA
aagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttctgtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgtcgctgtcagacacgatcaattcagatactacggttacgcctccgttattagcttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 44 - DNA, 876 bp, synthetic construct
  source 1..876: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 45 - DNA, 1087 bp, synthetic construct
  source 1..1087: mol_type = other DNA
gaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtg

SEQ ID NO: 46 - DNA, 1257 bp, synthetic construct
  source 1..1257: mol_type = other DNA
gaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggaacgactttagatgataccgcctaccgttataggacatcagtaccgggcgatgcagaagcaattgaagcccttgatggatcattcaccacagacacagtttttcgcgtgacagctactggtgacggatttactctccgtgaagtgcctgtagatccgccattgacgaaagtatttccagatgatgagagtgatgacgagagcgatgatggcgaagatggtgatcctgactcgcgcactttcgtcgcatacggtgatgatggtgacctggccggatttgttgttgtgtcctactcggggtggaaccgtagattgaccgtcgaagatatcgaagtcgcgccagaacatcgtggtcatggcgtgggacgagcactgatgggtctagctacggaattcgctcgagagcgtggggcgggtcacttatggcttgaagttaccaatgtaaatgctcctgctattcacgcttatcgccgaatgggtttcaccttgtgtggcctcgacacggcattgtat

SEQ ID NO: 47 - DNA, 1049 bp, synthetic construct
  source 1..1049: mol_type = other DNA
attgaagcccttgatggatcattcaccacagacacagtttttcgcgtgacagctactggtgacggatttactctccgtgaagtgcctgtagatccgccattgacgaaagtatttccagatgatgagagtgatgacgagagcgatgatggcgaagatggtgatcctgactcgcgcactttcgtcgcatacggtgatgatggtgacctggccggatttgttgttgtgtcctactcggggtggaaccgtagattgaccgtcgaagatatcgaagtcgcgccagaacatcgtggtcatggcgtgggacgagcactgatgggtctagctacggaattcgctcgagagcgtggggcgggtcacttatggcttgaagttaccaatgtaaatgctcctgctattcacgcttatcgccgaatgggtttcaccttgtgtggcctcgacacggcattgtatgacggtactgcttctgacggggagcaagcgctatacatgtctatgccctgcccctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 48 - DNA, 1199 bp, synthetic construct
  source 1..1199: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 49 - DNA, 1621 bp, synthetic construct
  source 1..1621: mol_type = other DNA
tattctggtacgagccatcccaaaaatggatcatgacagctgccaagagccaggactataagattgagatctacagcagcgatgacctcaagtcctggaagctcgaaagcgcctttgcgaacgaaggctttcttggctatcagtatgagtgccccggcctcatcgaggtgcctaccgagcaagatcctagcaagagctactgggtgatgttcatctctatcaatccgggagcacccgcgggcggctccttcaaccagtattttgtgggttcgttcaacggaacgcactttgaagcttttgataatcagtcaagagtcgtcgactttggcaaagactactacgccctccagaccttctttaacacggatccaacgtacggttcggcactgggcatcgcgtgggcgtcgaactgggagtatagcgcctttgtccccactaacccttggcgctcgtcgatgtcgttagtgcgtaagttctcgctgaacaccgagtaccaggcaaatccggagacagaactaatcaaccttaaggcagagcctatcttgaacatctctaacgccggaccctggtctaggttcgccaccaacaccacgctcactaaggccaatagttacaatgtcgatctaagcaattcaacgggcacactggaattcgagctcgtttacgcagtcaacaccacccagacgatctcgaaatccgtctttgcagatttgtcgctgtggttcaagggccttgaggacccggaagagtacctccgcatgggttttgaagtctcagcgtcatcgttcttcctcgaccgcgggaattctaaggtaaagttcgtgaaggaaaacccctactttaccaatcgtatgtctgtgaacaatcagccattcaaatcggagaacgacctgtcctactacaaggtttacggccttctcgaccaaaacatcctcgagttgtactttaacgacggtgatgttgtgtcgacaaacacttatttcatgacaacgggaaacgctctcggatccgtcaatatgactacgggtgtcgacaatctgttttacattgacaaattccaggtgcgagaagtcaagtaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttat

SEQ ID NO: 50 - DNA, 1903 bp, synthetic construct
  source 1..1903: mol_type = other DNA
ataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataagcgtccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttaagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttctgtcatgacttgatttcccacatctatgctgctgatggttctgccgacgtcatgagagacatttctgcctctggtgagccagccatccctaac

SEQ ID NO: 51 - DNA, 1475 bp, synthetic construct
  source 1..1475: mol_type = other DNA
ataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 52 - DNA, 1982 bp, synthetic construct
  source 1..1982: mol_type = other DNA
ataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgttgcttcaagctttcctttttctgctcgctggtttcgccgctaagattagcgctagcatgacgaacgaaacctctgaccggccgttggtccacttcacccccaacaagggttggatgaacgacccaaacggcctctggtatgatgagaaagacgctaagtggcacctgtacttccagtataaccctaacgatacagtttggggtacgcccttgttttggggccatgcgacctccgacgacctcaccaactgggaagatcagcctatcgcgattgctccgaagcgtaacgattcaggtgcctttagtggcagtatggtcgtagactacaacaacaccagcggtttcttcaacgacactatcgatccacgacagcgctgtgttgccatatggacctacaacacgccggagtccgaggaacaatacattagttactcgctggacgggggatacacctttacagagtaccagaaaaatcccgtcctcgccgcaaactcgactcaattccgcgatcctaaggtattctggtacgagccatcccaaaaatggatcatgacagctgccaagagccaggactataagattgagatctacagcagcgatgacctcaagtcctggaagctcgaaagcgcctttgcgaacgaaggctttcttggctatcagtatgagtgccccggcctcatcgaggtgcctaccgagcaagatcctagcaagagctactgggtgatgttcatctctatcaatccgggagcacccgcgggcggctccttcaaccagtattttgtgggttcgttcaacggaacgcactttgaagcttttgataatcagtcaagagtcgtcgactttggcaaagactactacgccctccagaccttctttaacacggatccaacgtacggttcggcactgggcatcgcgtgggcgtcgaactgggagtatagcgcctttgtccccactaacccttggcgctcgtcgatgtcgttagtgcgtaagttctcgctgaacaccgagtaccaggcaaatccggagacagaactaatcaaccttaaggcagagcctatcttgaacatctctaacgccggaccctggtctaggttcgccaccaacaccacgctcactaaggccaatagttacaatgtcgatctaagcaattcaacgggcacactggaattcgagctcgtttacgcagtcaacaccacccagacgatctcgaaatccgtctttgcagatttgtcgctgtggttcaagggccttgaggacc

SEQ ID NO: 53 - DNA, 4286 bp, synthetic construct
  source 1..4286: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactctggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgaaagcttccatgctggaggacttgaacaaattcagcgtcaaagagatcgacatcccgtcccccaaaaaggatgaggtcgtcgtgaaggtgatggcggcaggcacgtgtggatccgattcgcataaaatgattagcggatggaaatatggctatcccgcggtgatgggacacgaattttctggaattgtgacacaactgggagaaaacgtgtccaacgtcagcgtcggtcaacacgtcgccgtggctcccttcattccctgcttcaagtgtcattattgtcaaatcggactcttccagatgtgtgaaaattattccatgctcggtcagcaaaaatttggaggtttcgagcaatacgtttccgtcccggcacgtaacgttctggacatcggcaagatgtcgttcgaggaaggtgcccttattgaaccgatggcagttgcggcccacgctgttatgggaattaaaccggagctgggtgatacagttgcggtcttcggcctcggtacagtcggagatcttgttgtccgtcttcttatttcctcgggagcgacaaacgtcattggcattgacattgacgaccagaagcttgaaaagggactggatgagggttgcacacacgtgatcaactctgccaaagagtctcttgaagaaaagatcatggaatacaccgacggtctgggtgtcgatatttccatggaatgcgccggcagcaaaatcactgaggagcaaacactgctcgttacgaaacgccgtggaaaggttggatttgtcggtattgcttatagcgacgttctgctccatcagaaagcatttgagaacatcttccgccacgagcttacggtgactggattttggaattcctattccgcaccatttcctggacgcgagtggaccaactcgatccaattggttaatcgtggtcgtatcaagatcaaagatctcattactcatcgatttgagcttgaagacatgcaaaaagcattcaatatgatcaccactcgttcggaatcgtttaataaggtcatgtttttccccaacggcatcaattaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcagattgttgcatcgtctcatcagatcgtccttcattgtagtgataattggtgcaaccttgtattagactgtatagcagaatccctcgctataaaaagtattatgcatacgcagttccaagcaggaattagtatggtagtgggacttggctgaagggctgacgacatggcgagaaaaacgtatcaattcggaacacactgtgcaatgtcgatcacgttttcggtggttcacggtaggcttcctagtccatgggaaagtggtaacatctatcccttgagcgcttgagcttaatgttaccttgtgcaaaagtcatttgaagcttcacttgaagcttgaagctttgaagttttgaagcttgaagcgcaaacagcagcggtacatgtatactggcgtaacctgcctacagtggtgcatcagtacgtcaagcgggtagcccgaaatctcattaaggtcatttgccctctaagcttagtacctagtgatctgttc

SEQ ID NO: 54 - protein, 231 aa, Moniliella pollinis
MPVCLIAPSILASDMGALTAECKRMLDCGADWLHMDVMDGHFVPNIVLGAPILASISKSLPDAELDCHMMVSAPEKWIKDIAAAGGKSYTFHLEATNDPLDTVRQIKASGMRAAVAINPGTPSAEISDELASQLDMILVMTVWPGAGGQKFIGECMPKVTELRRRFPDLNIEVDGGVGPKTIEKCAKAGANVIVAGTAIFHAERPRDVIHFLRTQVNEAQAQLKEEWAKSG

SEQ ID NO: 55 - DNA, 4064 bp, synthetic construct
  source 1..4064: mol_type = other DNA
gcgaggagtagttactaggtagcggtatagcaattccgcccataatgcataaccgttctccctaccaaaacaaaacaatggctacgtggccggcgtgcgtggccgacatggtgcgggtgcggggggcggggtacgagtgtggggagttcactagttcgctggctagctgactagctgaccggcgtgtggttgtggcagtacgcagtgggaaagattaaatatgaaatattgaatatcaaaatgacgtttggggtggcacgtgcgcgcgccaacaacaacaagagcagccaccaagccaaagcaccaagcaccaaccccgcacaccattttgttacacccgcggcagaaatggcgtcagccagcagtcactattactcaatattcaataggggttccacttgactcacttcctcatgtatatatatatccatctaatcttcccaatacataaccgtcatttagctacgatggtgtgcgtgtaactgatcagtacacccgctgtatcaaaactctatcttcaatccgctaagaatcgtcaccatcagtacatcagtacatcagtgggctaagatctaatggtgtcgttcgacggccgcgggcactagccacgagccaagagcggtacagttgctgcgaaatggtgggcagcctgacaggttcagtttgaataggatgaaaacgccatgaagaacagagaagctggcaattgacgatagtggggcgagcgcacgctgcttgctggtccgagcgtaggacgaacgacaaaccatgggcggggccaccaggccccagggagggcgtcactggttggatgtcgtctgttaggctagagctcaggcgctcagggcctctggcccaagcctcggacgcaagcctctgacgagcgagcgcgcgctggggcgggtctgcgcaggaacggcaggactgccgagttggttgaatattgaatggttggatgattggtttgctgggtttggttttgacacgcacggcacaggacaagtgagtttggaggttggatttgcgcaggtagaaagggcgctcggcctcagggcctcagcgtcagcagacgagctcgccagtccaggtcagtacccagtgttcgttacttcccaccgtccagagtttgctgctgcctctcagcttgattcgtgtcggagaagtagaaatggtcacagcgtaccacacactgtccccatgcctgcgtcctgctccacgagtgtttagccacaaaaatttatggacaaccttactgagcctgtgctggggggtgctttagcaccagcactggcgccatggcgtcaaggttaattccactgtcattcgacaaccttcaactgatgggggcaactgggcacaaaaccttggatatataaccaactccaactcccacggttttttaccttttgctcttctccccaggactcactacttctactcctctattcatcttcaccaaaatgccagtctgtctgatcgctccttccattttggcctcggacatgggagcactcacagccgagtgtaagcgaatgcttgactgtggtgctgattggctgcacatgggtacgttgccgtttcttgtgcgtacagaggtattgattgtttttatttatttattgacattgttatactacttctattctggcttgggatgcttgtgcagacgtcatggacgggtaagttctagtttgagcggaaagggtggctggggagaagggcacgagaataccgcctgcaaattgagtactgactgagtttggattctttttttgtttgccagccattttgtcccgaacattgttcttggggcccctatccttgcttcaatctctaaatctttacccgatgccgaactggattgccatatgatggtatctgctcctgaaaaggtacgtgtattaggtcccaaccaaaacttttgctgatggattcctgacatgcctctctctctccccccctccctctgcatacaccagtggatcaaagatatcgctgctgcaggcggcaagtcttacacttttcatctggaagcaacaagtacgatgacttttccttcctttcgagactcaagcagtgaactaaccatttgattggcacagatgaccccttggacactgtacgacagatcaaagcatccggtatgcgcgcagctgttgctattaacccaggaaccccctcagccgaaatttctgatgaactcgctagccaattggacatgatcctcgtaatgactgtctggccgggagctggtggacaaaagtttattggggaatgtatgcctaaagtaaccgaactacgacgtcgattccccgatttgaatattgaagtcgatggaggagttggacctaaaacgattgagaaatgtgccaaagctggtgccaatgttattgtggctggtactgctatcttccatgctgaacgtccaagggacgtaattcatttcctacgaacgcaagtgaatgaagctcaggctcaactgaaagaggaatgggccaagagcggttgataagttgctccctttgctgttctatgatccaaaagatgcttagtgctccactctctactcccgactaccttttacgcattctgacatgactagtagtgcaatgttacccctggtgttttctcgcaatcactttctgtctgctatagcttttgatcgtcagtagttttttggtatagctaaagtagacttttgtacctggcgttcaatgtaattttactccttttttgtgcgatgcaacagatggtgtaaagatcgtcgttaaagttttggcttgatgcttgattcggtgacggttgcttgggtgatccttatatcgtctcctggtgttctcataagcccaggctcttcttagctatgtacaacaggtccaccactgactatgttatgtagaggcatgcatttaacttacggaaagatttaatggagaagaacgtacctagtcagatgcttgacatgaaaacgtattcctgcaattcacaaatcagtatcctgcctgtccactcgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatggcaaagttgacgagcgccgtgccggtgctaaccgcgagagatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctggg

SEQ ID NO: 56 - DNA, 1375 bp, synthetic construct
  source 1..1375: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttataactttgagttcagtttgggaactatttaaacctgcttgctggtcacattattgtcagtacctaccatgaagaattcaatattattttacattgtcaaccattacatggatcccatgttgtgcatagcctctatgtcatcctttggtctcttatgtctgtagcgtgctagtagtattcaagttgtagcactgctgtgaaacagtacgttcaagtagtgtgcagcagtacgttcagttagtggactggggcagcctcgccttcagcaaaggttaggtacatgagccagttgctcactcttgctttctacgaatcccataggctactttgccccctaaccgtgacgaccccctgtgtttgagaagtttgagtctttttacgttaccgtgaccaagcgagcagagtagaaactcttgaagttgtgtatgtaagaatacaaattgagaacacaaggttacctacctttatagttggtaaacagcaagcaataccagagatttt

SEQ ID NO: 57 - DNA, 3754 bp, synthetic construct
  source 1..3754: mol_type = other DNA
agatgtagccggcgctgtagagttttggacggataggttgggtttcagtcgagacttcgttgaagatgactttgcaggcgtcgtgcgtgatgatgtgaccctgtttatctcggccgttcaagatcaagttgtccctgacaatacacttgcatgggtctgggtacgcggattagatgaattgtacgcggaatggtctgaagttgtatccactaactttcgtgacgcttcaggtcctgctatgactgagattggtgaacaaccctgggggcgtgagttcgcactccgagatccagctggaaattgtgttcatttcgtagctgaggaacaggattaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttatgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatggttaccgagatcgaagtggatgcggtgctgttcgacatggacggcaccctgattgactctacccctgctgtcaaccagacttgggccgagtacatcgaacgatacaacctcgactttgactacgtcatgtctcattgtcacggatgtcgaacggtagagagcctcaagaatatcgttggattgccggatgaccagttggaacaaggcgtagtcgagtttgaagggcgtattagtgaactggctgaagaggcggcacgtaccaatggtcctggtcaaattgtggctttgcccggcgcaaaagaactgcttgcccaactgaacaagggtagcgaacaacctggtcgtcgtcctggatgggccatcgttacttcggcaaccaaggcttacgcttacgtgggcttcagagcgtcgggcgtgaatggtacagaattccctcgtgcttttgtcacgggtgacaatgtcagcaagggcaaacctgatcctgaaccatacctcttgggagcaaagacgttacaacctcctgctgatcctaaacgttgtctcgtaatcgaagatgctcctccaggtattcgatcaggtgttgctgcaggtgccaaaacccttggattgaccactacccacgcggaaaacctcatgtggcaagcgggtgctaactgggtcgtaccagacttatcgagtgtttcggcacgttgggaaggtgaaggtgcggacgctcgtctttttattcgcattgaaaactcaggccagcgtccagagtaacacggatcaacaaaaatgtgcgccagctttgttgtgtgcccttttcctctagcggatcctatccacatttcgtgaatgaaccgattaagctctgacgagatttccttgttttctgtagatacctcttgtagaggtgtcaaagttatgttgtgatcgagagagttacctgcaaggtattatgcaagcggcttgatgaggacactcaactacgtgtgagtaggagtgcgttgctcagcagttgacgcgtgagccgcttgagcagcttgataaaggtcctaatcgtcttccagctgttggcagacgacaatgtgctgtagtagtaggataggcccgtaaaagtgtagaagcaataggttgagactgactgtaatgggtacttgcgttcaaagactgaaagtccaaaggattgctcaaaggcccctgaggtctgagcctctgagcgcgctcttcgtcaaaattcgccggggccatcggtcatcctctcgagggcagcatcttcctgcttgaagactgagtccgtctcaggcttgctggtcacattattgtcagtacctaccatgaagaattcaatattattttacattgtcaaccattacatggatcccatgttgtgcatagcctctatgtcatcctttggtcttttatgtctgtagcgtgctagtagtattcaagttgtagcactgctgtgaaacagtacgttcaagtagtgtgcagcagtacgttcagttagtggactggggcagcctcgccttcagcaaaggttaggtacatgagccagttgctcactcttgctttctacgaatcccataggctactttgccccctaaccgtgacgaccccctgtgtttgagaagtttgagtctttttacgttaccgtgaccaagcgagcagagtagaaactcttgaagttgtgtatgtaagaatacaaattgagaacacaaggttacctacctttatagttggtaaacagcaagcaataccagagattttcatcgcaacgtgactctatcattcactatattaagttgcacacggatgttgtacaacactacacgtaaaagagagaaaaatactaatgtcgatcgctcaggt

SEQ ID NO: 58 - DNA, 1729 bp, synthetic construct
  source 1..1729: mol_type = other DNA
cttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttataagcttgaagactgagtccgtctcagttgctgctacgacctcattgatcgatgatctccgacactttgagtagctttccatcctctctttactgtactttgtattcttgctactttgtgttctttcttaatggtcccgaaccggtgattttgctcatgatctttttttttttttttcgtcaactgcaagaagatgaaaaatctactgttcgatagcacctcataaccgttccagagtcccgacaaccacaaggatgtttcttagataggtgtcagcaagatccacaaaaaagtgtaggtattagatggtttcaagcaaggtacaatattctaattcaatactcatttgaataatcaatattcaacagcttctagcctacaagcttcaatgttctccgtcggttaactcagtatacttagtactcagttcaagtcagtgttgttaaaataaaatcacgtgatccaaaaacttgaaacttttcatttttctcctcagatgttggtgattcgtcccatagcggttaccacaac

SEQ ID NO: 59 - DNA, 8167 bp, synthetic construct
  source 1..8167: mol_type = other DNA
accgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtcctgcaggtatcagatctcagatctcagagagatgtcagagcagacctcgcccttacaaagacacttgtacaaaagttttcaggcatacggattccttcccttcctcgcagaaactctctatcgcccctttgcacttgccgccgtttggtattggacaattgattgctagcaccccttgcaatgatgttatacaaatttatctactgaattgcttgagagtcagtaacaccacggcttccgcgcgagattccatcaccaggcggtggtcacacagatgagttcatcaacgggattctgtcaagtatctcgcatcaacaaatttagctaacaagtctgaacacgttctattcaaattcagaagccacaagaagataactttagtaggaaaagtagattgcaagcacactccgaggcttgctctcgggcgaagaagggatattttgggctcagagcgccatcagaacgacggacgctccaacaaaggggaagcaacagtggtgaacaccattaggggcagggcatagacatgtatagcgcttgctccccgtcagaagcagtaccgtcatacaatgccgtgtcgaggccacacaaggtgaaacccattcggcgataagcgtgaatagcaggagcatttacattggtaacttcaagccataagtgacccgccccacgctctcgagcgaattccgtagctagacccatcagtgctcgtcccacgccatgaccacgatgttctggcgcgacttcgatatcttcgacggtcaatctacggttccaccccgagtaggacacaacaacaaatccggccaggtcaccatcatcaccgtatgcgacgaaagtgcgcgagtcaggatcaccatcttcgccatcatcgctctcgtcatcactctcatcatctggaaatactttcgtcaatggcggatctacaggcacttcacggagagtaaatccgtcaccagtagctgtcacgcgaaaaactgtgtctgtggtgaatgatccatcaagggcttcaattgcttctgcatcgcccggtactgatgtcctataacggtaggcggtatcatctaaagtcgttcccatggtgaaagagtaccggtgtgttggtaaagagacaagaatgacaagaagatgaagcactagttatatagggccggcagagttgctgcatgctgcggcagtctaaccgaagaaaaaatttgcacatgacgacccaggcactggcccacagtcacttcaactcccctcgtaatgcattttctgactggccttatgcctcgtgtttctgttttctttctcagcttgcgttgaatcttgggaacaaccctgccaatctaactaaattaaccaattcacccacttccgcttccgtcagcttttttccgcaccctcgatgtgcatcgaagttcgacaaactatagtgagatcgatctcgatcgtaatcggtaaagttgtgtagtatattggctattacaagaagcatccctggggaagggtctgcaggtgtcatggcctagtacagagggcaatgattaatatggatagtgacgcatgatgcatgatgcatgatgcatgatggatgcatagggcaaagcgaaccaatgccatctgcgtcatctgcatcgatgatcgatgtatctactacgtactatggcaaacaggaaacattggatacatggcaagatgacaatggtaacgtgcaatcttgtgcgaattagtggtcatagaggcaaggtcaaggctaacatgcaaggtctgagctcctgcaggactagtaacggccgccagtgtggcgcgccattgtcattcgtgattctagtactgtgggtggctgcgaaactccttcactcaatgaattacaatcatgattcgtgattcgcaccgatcccaccagcctctcgagaaaagcggacttgaacattcatgattcacgaccctgtgattcatgaatgccatccgatccagaacggacttgctactcacgactcagactgaatttttaaatgccctcatgatttgattcatgatcagcaatgccctcatgatttgattcatgattcgctgatcacagcggtgaagaacgtaacccattcacgattcagaaggcaagtactgcttcttgctcggctgctttgttgctgccgaggcgggctagcttgcacaaagatcgaggatcggtaatggcgcgccaggcggccgctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaatgtccaacttgctcaccgtccaccagaacctcccggccctgccagtggacgcgaccagcgatgaagttcgaaaaaatcttatggatatgttccgcgatcgacaagccttttccgaacatacctggaagatgctgttgagcgtgtgccgatcgtgggccgcttggtgcaaactgaacaaccgcaagtggtttcccgccgagccggaagatgttcgtgattacctgctctacctgcaagcacgtggtttggcggtgaagactatccaacagcacctgggtcaacttaatatgcttcaccgccgatctggactcccacgtccctctgactccaacgctgtgtcccttgttatgcgtcgtattcgcaaagaaaacgtggacgctggcgaacgagcgaaacaggctttggcatttgaacgaactgacttcgatcaggtccgctccttgatggaaaactctgaccgttgccaagacatccgtaacctcgcgttcctgggaatcgcctacaataccctcctccgtatcgctgaaattgctcgcattcgagtgaaggacatttctcgaacagacggcggccgtatgcttattcatatcggtcgtactaaaacactcgtcagcactgctggagtcgagaaagcgctctctcttggagttactaaattggtggaacgatggatttctgtttctggagttgcggatgatcctaataactacttgttttgtcgtgttcgtaagaacggcgtcgccgcgccctccgccacatcccagctctccacacgtgcactcgagggaatcttcgaggcgactcaccgtctgatttacggcgctaaagacgatagcggtcaacgataccttgcctggtctggtcactccgcacgagtgggcgcagcccgcgacatggcgcgtgcaggagtgtccattccagaaatcatgcaagcgggcggatggacgaacgtcaacatcgtgatgaattatattcgtaacttggactcggaaaccggagcaatggtccgacttctcgaggatggcgattaagttgctccctttgctgttctatgatccaaaagatgcttagtgctccactctctactcccgactaccttttacgcattctgacatgactagtagtgcaatgttacccctggtgttttctcgcaatcactttctgtctgctatagcttttgatcgtcagtagttttttggtatagctaaagtagacttttgtacctggcgttcaatgtaattttactccttttttgtgcgatgcaacagatggtgtaaagatcgtcgttaaagttttggcttgatgcttgattcggtgacggttgcttgggtgatccttatatcgtctcctggtgttctcataagcccaggctcttcttagctatgtacaacaggtccaccactgactatgttatgtagaggcatgcatttaacttacggaaagatttaatggagaagaacgtacctagtcagatgcttgacatgaaaacgtattcctgcaattcacaaatcagtatcctgcctgtccactctctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaa

SEQ ID NO: 60 - DNA, 500 bp, Moniliella pollinis
  source 1..500: mol_type = other DNA
tatcagatctcagatctcagagagatgtcagagcagacctcgcccttacaaagacacttgtacaaaagttttcaggcatacggattccttcccttcctcgcagaaactctctatcgcccctttgcacttgccgccgtttggtattggacaattgattgctagcaccccttgcaatgatgttatacaaatttatctactgaattgcttgagagtcagtaacaccacggcttccgcgcgagattccatcaccaggcggtggtcacacagatgagttcatcaacgggattctgtcaagtatctcgcatcaacaaatttagctaacaagtctgaacacgttctattcaaattcagaagccacaagaagataactttagtaggaaaagtagattgcaagcacactccgaggcttgctctcgggcgaagaagggatattttgggctcagagcgccatcagaacgacggacgctccaacaaaggggaagcaacagtggtgaacacca

SEQ ID NO: 61 - DNA, 1106 bp, Moniliella pollinis
  source 1..1106: mol_type = genomic DNA
atgaccgacacaattgacgttgctgctttccaagcacacaaggctttccaaggcgaccaggagtcgtaccgtaagggctcgaaccgtcctggtttgcttgatggtgagcgtcgtcgctcatcggtctctgatgccaagatcaagctcaactcgggtcactacattcccgtcgtcgcgcttggtgtgtacaaggcacctaacgattcgactaccgaggatgccgtcaagtgggcttttgaggctggataccgtcatgttgattcgggtatgttacggttctctcccttgacctcttgatctctttgatctggtagctgaccattcatgaattgcaatctccagctgctcgatacgccaatgaggaagctgttggccgtgcattgaatgactttgtcaagactaccggtgtcccccgcgaggaaatctttgttacgtctaagctttgggacgcagaccacgaacgtgctgacgctgcgattgatgaatccttgcaaaagctcggtttgacgtacatggacttgtacctgttgcattcgcccggtgctgaccgtgacactcggttgaaggcttggaaagctatggaagatgctgtgtacgctggtaagatcaagtccattggtgtttccaactttgatgtagaagagttggatgacttgttggccaactgccgtattaagcctgctgtcaatcaaattgaatcgcatccattcttccaacatgaagatttgcgcaaagctaccattgaacgtggtattcacgtacaagcatattcgcctatggcacaaggtgctgcattggaacgtcctgagattgttgctattgccaagaagcacggtaagagtactgcacaaattttgctcaagtggggtatcaacattggtaacattgtgttacctaagagtttgactcgatcgcgtattgaagccaatgcagacttgtttgactttgaattggatcaagatgatatggatgtactgaacaatttggatgaaggtatgaagatgggcaaattgggtcctgctggtactagtgctcctggatcgcgtcgtacatctagtaactcgggtggcttgtctggtggtaaattacaaatgagttccaagtaa

SEQ ID NO: 62 - DNA, 1026 bp, Moniliella pollinis
  source 1..1026: mol_type = other DNA
atgaccgacacaattgacgttgctgctttccaagcacacaaggctttccaaggcgaccaggagtcgtaccgtaagggctcgaaccgtcctggtttgcttgatggtgagcgtcgtcgctcatcggtctctgatgccaagatcaagctcaactcgggtcactacattcccgtcgtcgcgcttggtgtgtacaaggcacctaacgattcgactaccgaggatgccgtcaagtgggcttttgaggctggataccgtcatgttgattcggctgctcgatacgccaatgaggaagctgttggccgtgcattgaatgactttgtcaagactaccggtgtcccccgcgaggaaatctttgttacgtctaagctttgggacgcagaccacgaacgtgctgacgctgcgattgatgaatccttgcaaaagctcggtttgacgtacatggacttgtacctgttgcattcgcccggtgctgaccgtgacactcggttgaaggcttggaaagctatggaagatgctgtgtacgctggtaagatcaagtccattggtgtttccaactttgatgtagaagagttggatgacttgttggccaactgccgtattaagcctgctgtcaatcaaattgaatcgcatccattcttccaacatgaagatttgcgcaaagctaccattgaacgtggtattcacgtacaagcatattcgcctatggcacaaggtgctgcattggaacgtcctgagattgttgctattgccaagaagcacggtaagagtactgcacaaattttgctcaagtggggtatcaacattggtaacattgtgttacctaagagtttgactcgatcgcgtattgaagccaatgcagacttgtttgactttgaattggatcaagatgatatggatgtactgaacaatttggatgaaggtatgaagatgggcaaattgggtcctgctggtactagtgctcctggatcgcgtcgtacatctagtaactcgggtggcttgtctggtggtaaattacaaatgagttccaag

SEQ ID NO: 63 - protein, 342 aa, Moniliella pollinis
MTDTIDVAAFQAHKAFQGDQESYRKGSNRPGLLDGERRRSSVSDAKIKLNSGHYIPVVALGVYKAPNDSTTEDAVKWAFEAGYRHVDSAARYANEEAVGRALNDFVKTTGVPREEIFVTSKLWDADHERADAAIDESLQKLGLTYMDLYLLHSPGADRDTRLKAWKAMEDAVYAGKIKSIGVSNFDVEELDDLLANCRIKPAVNQIESHPFFQHEDLRKATIERGIHVQAYSPMAQGAALERPEIVAIAKKHGKSTAQILLKWGINIGNIVLPKSLTRSRIEANADLFDFELDQDDMDVLNNLDEGMKMGKLGPAGTSAPGSRRTSSNSGGLSGGKLQMSSK

SEQ ID NO: 64 - DNA, 1079 bp, Moniliella pollinis
  source 1..1079: mol_type = genomic DNA
atggcttcttatcacgagtacgtccattcctatttctgatcgagcctttgtcagtttggaaaatctccagaattcctaaccttggttgtttcccatttgctcttgctgccttcagacccgttaaagaatttaccttggctgacggtaacaagattccttccattggattgggaacttggcaatcagctcctggtgaagtcgctgctgctgttgctcatgcaatcaaatctggttaccgtcacattgatgccgcttgggtctatggtaacgaacatgaagttggtcaaggaattaaagattctggtgttccccgtgatcaaatctttgtaacgtccaaatgttttgaaattcatcattctaatcctgaagctgcgttaaaagatacgttaaccaatttaggtttggagtatttggatttgtatttgatgcattggcccttagcgtacgaaccagttcctacgagcgatggatcgttacctaaagaagttaagaaggtggatggcaaacctgttttggataagattcaaaccgaagatgttgtaccatgctggcaaaaaatgcaacaattggttaagcagggattaaccaaaagcattggaatttccaactttaacattcggaagactcgtcacttgttacaacatgctgaaattaaacctgttgtgaaccaaatcgaagtcaactttggtaatcctcaaccggatttgattcaatggttacaacgtcatcagattgtggtcgaagcgtattccccacttggttccaccggagccaaatacaatgattctcctgtatttggagaagtagctagcaaacggaatgtgagccctgctaccgtaaccatctcttggttattacaacgtggtttggtcgttttacccaagagtgttactccttctcgtattgagagtaactttaactttattacgttgaccaaagatgaatttgaagcgattgaaaaagctgcttgttctcatcctggttctcgcgtttgtgaccaaagtgaattggccagctacgatatctttgaagataagcaccctgaatacaatgatcaagctcaagcgaaattggattaa

SEQ ID NO: 65 - DNA, 978 bp, Moniliella pollinis
  source 1..978: mol_type = other DNA
atggcttcttatcacgaacccgttaaagaatttaccttggctgacggtaacaagattccttccattggattgggaacttggcaatcagctcctggtgaagtcgctgctgctgttgctcatgcaatcaaatctggttaccgtcacattgatgccgcttgggtctatggtaacgaacatgaagttggtcaaggaattaaagattctggtgttccccgtgatcaaatctttgtaacgtccaaatgttttgaaattcatcattctaatcctgaagctgcgttaaaagatacgttaaccaatttaggtttggagtatttggatttgtatttgatgcattggcccttagcgtacgaaccagttcctacgagcgatggatcgttacctaaagaagttaagaaggtggatggcaaacctgttttggataagattcaaaccgaagatgttgtaccatgctggcaaaaaatgcaacaattggttaagcagggattaaccaaaagcattggaatttccaactttaacattcggaagactcgtcacttgttacaacatgctgaaattaaacctgttgtgaaccaaatcgaagtcaactttggtaatcctcaaccggatttgattcaatggttacaacgtcatcagattgtggtcgaagcgtattccccacttggttccaccggagccaaatacaatgattctcctgtatttggagaagtagctagcaaacggaatgtgagccctgctaccgtaaccatctcttggttattacaacgtggtttggtcgttttacccaagagtgttactccttctcgtattgagagtaactttaactttattacgttgaccaaagatgaatttgaagcgattgaaaaagctgcttgttctcatcctggttctcgcgtttgtgaccaaagtgaattggccagctacgatatctttgaagataagcaccctgaatacaatgatcaagctcaagcgaaattggat

SEQ ID NO: 66 - protein, 326 aa, Moniliella pollinis
MASYHEPVKEFTLADGNKIPSIGLGTWQSAPGEVAAAVAHAIKSGYRHIDAAWVYGNEHEVGQGIKDSGVPRDQIFVTSKCFEIHHSNPEAALKDTLTNLGLEYLDLYLMHWPLAYEPVPTSDGSLPKEVKKVDGKPVLDKIQTEDVVPCWQKMQQLVKQGLTKSIGISNFNIRKTRHLLQHAEIKPVVNQIEVNFGNPQPDLIQWLQRHQIVVEAYSPLGSTGAKYNDSPVFGEVASKRNVSPATVTISWLLQRGLVVLPKSVTPSRIESNFNFITLTKDEFEAIEKAACSHPGSRVCDQSELASYDIFEDKHPEYNDQAQAKLD

SEQ ID NO: 67 - DNA, 1122 bp, Moniliella pollinis
  source 1..1122: mol_type = genomic DNA
atggcagctgataccattgacgttgctgctttccaagcgcaaaaagaatttcaaaatcaacaaggatcttatcgtaaagcagaaaatcaccctggattgctcgatggtgaacgtcgtcgttcatccgtaagtgaggccaaagtgaagctcaactcgggtcattacattcccatggtagcactcggtgtgtacaaggcaccaaatgactcatctactgaggaaacgatcaaatgggcgtttgaatctgggtaccgtcatgttgattcaggtacgtttcctaatcgctttcctttgatctttttcatctaacaaggaagaagacttgaaagtactgactttgaaattatcctgttgacagctgcaaggtacgctaatgaagaagctgtcggtcgtgctgtaaatgattttgtcaagactgccggcgtaccacgggaagaaatcttcgttacgaccaaattatgggatgaagatcatcatcgggccactgaagcaattgatgaatccttcaataagcttaatacgggatacattgatttgtatttacttcattcccctggtcctgatcgagatatgcgtcttcaagcttggaaagctatggaagatgctgtatacgcaggaaaaatcaaatcaattggtgtttccaactttgatgtggatcaattggatgatcttttggctcatgcacgaattaaacctgctgtgaaccaaattgaatctcatcctttcttccaacatgaagatttacgtcgagctactgctgaacgtggaattcatgtagaggcatactctcctatggcgaaaggagctgcattggaacgtcctgaaattgtcaacattgctaagaaacatggtaagagtactgcacaagtattactcaagtggggtatgcaagttggtaatatcattttgcctaaaagtttgaacaaatctcgaatcccatccaatgccgatctgttcgactttgaattggatcaagatgatatggatgtattgaacaatctggatgaaggtatgaaaacgggtaaattgggtcctttgggtactagtgctccaggatcacgtcgtacatcgtccaacagtggtggagccagtggtgctaaactccaaatgagttctacgtaa

SEQ ID NO: 68 - DNA, 1029 bp, Moniliella pollinis
  source 1..1029: mol_type = other DNA
atggcagctgataccattgacgttgctgctttccaagcgcaaaaagaatttcaaaatcaacaaggatcttatcgtaaagcagaaaatcaccctggattgctcgatggtgaacgtcgtcgttcatccgtaagtgaggccaaagtgaagctcaactcgggtcattacattcccatggtagcactcggtgtgtacaaggcaccaaatgactcatctactgaggaaacgatcaaatgggcgtttgaatctgggtaccgtcatgttgattcagctgcaaggtacgctaatgaagaagctgtcggtcgtgctgtaaatgattttgtcaagactgccggcgtaccacgggaagaaatcttcgttacgaccaaattatgggatgaagatcatcatcgggccactgaagcaattgatgaatccttcaataagcttaatacgggatacattgatttgtatttacttcattcccctggtcctgatcgagatatgcgtcttcaagcttggaaagctatggaagatgctgtatacgcaggaaaaatcaaatcaattggtgtttccaactttgatgtggatcaattggatgatcttttggctcatgcacgaattaaacctgctgtgaaccaaattgaatctcatcctttcttccaacatgaagatttacgtcgagctactgctgaacgtggaattcatgtagaggcatactctcctatggcgaaaggagctgcattggaacgtcctgaaattgtcaacattgctaagaaacatggtaagagtactgcacaagtattactcaagtggggtatgcaagttggtaatatcattttgcctaaaagtttgaacaaatctcgaatcccatccaatgccgatctgttcgactttgaattggatcaagatgatatggatgtattgaacaatctggatgaaggtatgaaaacgggtaaattgggtcctttgggtactagtgctccaggatcacgtcgtacatcgtccaacagtggtggagccagtggtgctaaactccaaatgagttctacg

SEQ ID NO: 69 - protein, 343 aa, Moniliella pollinis
MAADTIDVAAFQAQKEFQNQQGSYRKAENHPGLLDGERRRSSVSEAKVKLNSGHYIPMVALGVYKAPNDSSTEETIKWAFESGYRHVDSAARYANEEAVGRAVNDFVKTAGVPREEIFVTTKLWDEDHHRATEAIDESFNKLNTGYIDLYLLHSPGPDRDMRLQAWKAMEDAVYAGKIKSIGVSNFDVDQLDDLLAHARIKPAVNQIESHPFFQHEDLRRATAERGIHVEAYSPMAKGAALERPEIVNIAKKHGKSTAQVLLKWGMQVGNIILPKSLNKSRIPSNADLFDFELDQDDMDVLNNLDEGMKTGKLGPLGTSAPGSRRTSSNSGGASGAKLQMSST

SEQ ID NO: 70 - DNA, 3881 bp, synthetic construct
  source 1..3881: mol_type = other DNA
gcctcctgctcgatacgtcgatacgtcgatgctggatgagtacatgggtacatgggtacatgggtactggatggtcgatggtcttctgtctggctcggcactgcctgactgggcaacgacgacgacgacgacgacgacgacgacgaccactggagccgtgctgcgatcgtcctaatgatcggggtggcaaggcacaccgtgcctgtgcctgacccacctgtgcacgacgtcatgggtcgcctgcctactagggtgtccgtcgcaggtcgcttagatgggaagcgggacgtgggacccctcgaccctcgattagctgcgcggccaccggccaccggccacttgcgtcaatgcgtcaatgcgtcaatgcgtcaacacggacctggtgcgttgacgcgctgacgtctaggcgcccaccggccaccacccaccagtacgcaatccagacccaggcagctccaagcttggggggttgggcagtacgccgtacggcgcggtcacagatctgagatctgcgacgctggcacggtgtccgcatcgcacgcgtcctaacacactgactgtatactgtatcctgtacccagcacccgatatccaatgcccagtactcagtgtacccaatgtactcactcagtgtacttactccgtactggctgcctgcggcctggcagacgggcgcctcagctcaaaccagcgcgatgcccagcccaacccattccattccatcccagtccagtctccagtccagtcgagccctgccctgccctgccctgccctgccctgcccggcacaccccaatcatctagcttagcgcccgcccacatatgccaacccccgagcgaggctcaatgttcgattctcgatgcgtgacgatgcttcgttacgctcggtcctccgcagccaggggttatataagtacgctcggtccgcttggtccgacatggtttgctcctaccaccatccgatccgtctactagctaccatcgtattctacccttctactgcactactgacagtcaaacaccttataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggaacgactttagatgataccgcctaccgttataggacatcagtaccgggcgatgcagaagcaattgaagcccttgatggatcattcaccacagacacagtttttcgcgtgacagctactggtgacggatttactctccgtgaagtgcctgtagatccgccattgacgaaagtatttccagatgatgagagtgatgacgagagcgatgatggcgaagatggtgatcctgactcgcgcactttcgtcgcatacggtgatgatggtgacctggccggatttgttgttgtgtcctactcggggtggaaccgtagattgaccgtcgaagatatcgaagtcgcgccagaacatcgtggtcatggcgtgggacgagcactgatgggtctagctacggaattcgctcgagagcgtggggcgggtcacttatggcttgaagttaccaatgtaaatgctcctgctattcacgcttatcgccgaatgggtttcaccttgtgtggcctcgacacggcattgtatgacggtactgcttctgacggggagcaagcgctatacatgtctatgccctgcccctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttataactttgagttcagtttgggaactatttaaacctgctttaaatgaggaaaaaggaattcatcctatgttcttctattccataccgttccactcaaaacttgaacaaggttgttgttccaagttgtttttttaccgaattctcctctccctctaccttttgatgtcttttctgatcaactagtaccactttctcggtttcttcctcttggttgcacaaccactctctgtgtaccgactttgaaaagacttgtatcattgcatgcgcttgaaggctatcaattatcattcatagtgggtttatagttttgtccctgccatccacaatgtaccatagaccatcgctgccatggctgcgcgttgcgcgctgccacccagaccacctttttccagtccgatacaaaatcatgttagtcaattgagcacgggccacccaagggtgacggtcacccctaaggctccgggagtagtaaggcactataccagtttcttttacctccatgcaatttcaaccatggtgcatcgcatctagaatctaggattctaggaatctaggaatctaggaatctaggatctggttaaccccaaaccatattaagacaggctcaatgggcagttagggttagcgtgcaaggagcgccgaggaaccggtcaggagatggggagagcaagctgcaaggtgagatgcccgaaatttgaaatttgaaatgtaaaaccccccgattggctatcgggctcggcgctggaagctggaattggttaggcacgacataagtctttaggcactaggctaggtaggcactagccgtcctcctatcctcgttgctgggcgctggaagctggaggcttcagaaattagtacgatagatactgactcagtactgtgcgcacccatgcgctcccagatcccagatcccagctggtagcagcttgggagcttgggagcttggcatgttggcaagccacatcatgcatccatcatgcaatcatatcttttcaccttgacaacttacattggttgtac

SEQ ID NO: 71 - DNA, 8404 bp, synthetic construct
  source 1..8404: mol_type = other DNA
accgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtcctgcaggtatcagatctcagatctcagagagatgtcagagcagacctcgcccttacaaagacacttgtacaaaagttttcaggcatacggattccttcccttcctcgcagaaactctctatcgcccctttgcacttgccgccgtttggtattggacaattgattgctagcaccccttgcaatgatgttatacaaatttatctactgaattgcttgagagtcagtaacaccacggcttccgcgcgagattccatcaccaggcggtggtcacacagatgagttcatcaacgggattctgtcaagtatctcgcatcaacaaatttagctaacaagtctgaacacgttctattcaaattcagaagccacaagaagataactttagtaggaaaagtagattgcaagcacactccgaggcttgctctcgggcgaagaagggatattttgggctcagagcgccatcagaacgacggacgctccaacaaaggggaagcaacagtggtgaacaccattagaaaaattcatccagcatcaaatggaactggagcttgttcatgtctgggttatcgatcccatatttctggaacagacgcttctggaggctaggggagaactcgcccagacaattccagaggatcgcaagatcttggtagcgatcggcgatgccaacccggccgacgtcaatgcagccaatgagcttgccctcgtcaaagatgaggttgtcaagcgagaaatcaccatgcgtcacgacagagtctggggaaaagggcaggagcttatgcatctctttccacacttgttccaccggccagccgttgcgctcgtcatcgaaatcactagcgtcgactagaccattgttcatgcgtgattgtgcctgggcaagcctaaaaacacggtcgctgttgaaagggcagttgcagaccggaatagagtgaaggcggcgcaaaaacacagccaaggcatccacgatgttctcgcccgagtcagggtattcctcgaggacctgaaaagcggtcttaccggggatggccgtagtcaacagccacgcatcgtcaggtgtacggatgaagtgcttgatggtggggagtggcatgaactcagttagccaattcagacggaccatttcgtcggttacgtcgtttgcaacgctacccttgccgtgtttaagaaagagctcgggggcgtccggttttccatagagtctgtagatcgtcgcacccgactgtccgacattatctcgagcccatttgtatccgtacaggtctgcgtccatgttggagttgagtcggggtcgcgatacgtgtgtcttttccttacccatggtgaaagagtaccggtgtgttggtaaagagacaagaatgacaagaagatgaagcactagttatatagggccggcagagttgctgcatgctgcggcagtctaaccgaagaaaaaatttgcacatgacgacccaggcactggcccacagtcacttcaactcccctcgtaatgcattttctgactggccttatgcctcgtgtttctgttttctttctcagcttgcgttgaatcttgggaacaaccctgccaatctaactaaattaaccaattcacccacttccgcttccgtcagcttttttccgcaccctcgatgtgcatcgaagttcgacaaactatagtgagatcgatctcgatcgtaatcggtaaagttgtgtagtatattggctattacaagaagcatccctggggaagggtctgcaggtgtcatggcctagtacagagggcaatgattaatatggatagtgacgcatgatgcatgatgcatgatgcatgatggatgcatagggcaaagcgaaccaatgccatctgcgtcatctgcatcgatgatcgatgtatctactacgtactatggcaaacaggaaacattggatacatggcaagatgacaatggtaacgtgcaatcttgtgcgaattagtggtcatagaggcaaggtcaaggctaacatgcaaggtctgagctcctgcaggactagtaacggccgccagtgtggcgcgccattgtcattcgtgattctagtactgtgggtggctgcgaaactccttcactcaatgaattacaatcatgattcgtgattcgcaccgatcccaccagcctctcgagaaaagcggacttgaacattcatgattcacgaccctgtgattcatgaatgccatccgatccagaacggacttgctactcacgactcagactgaatttttaaatgccctcatgatttgattcatgatcagcaatgccctcatgatttgattcatgattcgctgatcacagcggtgaagaacgtaacccattcacgattcagaaggcaagtactgcttcttgctcggctgctttgttgctgccgaggcgggctagcttgcacaaagatcgaggatcggtaatggcgcgccaggcggccgctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaatgtccaacttgctcaccgtccaccagaacctcccggccctgccagtggacgcgaccagcgatgaagttcgaaaaaatcttatggatatgttccgcgatcgacaagccttttccgaacatacctggaagatgctgttgagcgtgtgccgatcgtgggccgcttggtgcaaactgaacaaccgcaagtggtttcccgccgagccggaagatgttcgtgattacctgctctacctgcaagcacgtggtttggcggtgaagactatccaacagcacctgggtcaacttaatatgcttcaccgccgatctggactcccacgtccctctgactccaacgctgtgtcccttgttatgcgtcgtattcgcaaagaaaacgtggacgctggcgaacgagcgaaacaggctttggcatttgaacgaactgacttcgatcaggtccgctccttgatggaaaactctgaccgttgccaagacatccgtaacctcgcgttcctgggaatcgcctacaataccctcctccgtatcgctgaaattgctcgcattcgagtgaaggacatttctcgaacagacggcggccgtatgcttattcatatcggtcgtactaaaacactcgtcagcactgctggagtcgagaaagcgctctctcttggagttactaaattggtggaacgatggatttctgtttctggagttgcggatgatcctaataactacttgttttgtcgtgttcgtaagaacggcgtcgccgcgccctccgccacatcccagctctccacacgtgcactcgagggaatcttcgaggcgactcaccgtctgatttacggcgctaaagacgatagcggtcaacgataccttgcctggtctggtcactccgcacgagtgggcgcagcccgcgacatggcgcgtgcaggagtgtccattccagaaatcatgcaagcgggcggatggacgaacgtcaacatcgtgatgaattatattcgtaacttggactcggaaaccggagcaatggtccgacttctcgaggatggcgattaagttgctccctttgctgttctatgatccaaaagatgcttagtgctccactctctactcccgactaccttttacgcattctgacatgactagtagtgcaatgttacccctggtgttttctcgcaatcactttctgtctgctatagcttttgatcgtcagtagttttttggtatagctaaagtagacttttgtacctggcgttcaatgtaattttactccttttttgtgcgatgcaacagatggtgtaaagatcgtcgttaaagttttggcttgatgcttgattcggtgacggttgcttgggtgatccttatatcgtctcctggtgttctcataagcccaggctcttcttagctatgtacaacaggtccaccactgactatgttatgtagaggcatgcatttaacttacggaaagatttaatggagaagaacgtacctagtcagatgcttgacatgaaaacgtattcctgcaattcacaaatcagtatcctgcctgtccactctctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaa

SEQ ID NO: 72 - DNA, 4143 bp, synthetic construct
  source 1..4143: mol_type = other DNA
gcctcctgctcgatacgtcgatacgtcgatgctggatgagtacatgggtacatgggtacgggtactggatggtcgatggtcttctgtctggctcggcactgcctgactgggcaacgacgacgacgacgacgacgacgacgacgaccactggagccgtgctgcgatcgtcctaatgatcggggtggcaaggcacaccgtgcctgtgcctgacccacctgtgcacgacgtcatgggtcgcccgcctactagggtgtccgtcgcaggtcgcttagatgggacgcgggacgtgggacccctcgaccctcgattagctgcgcggccaccggccaccggccacttgcgtcaatgcgtcaatgcgtcaatgcgtcaacacggacctggtgcgttgacgcgctgacgtctaggcgcccaccggccaccacccaccagtacgcaatccagacccaggcagctccaagcttggggggttgggcagtacgccgtacggcgcggtcacagatctgagatctgcgacgctggcacggtgtccgcatcgcacgcgtcctaacacactgagtgtatactgtatcctgtacccagcacccgatatccaatgcccagtactcagtgtacccaatgtactcactcagtgtacttcctccgtactggctgcctgcggcctggcagacgggcgcctcagctcaaaccagcgcgatgcccagcccaacccattccattccatcccagtccagtctccagtccagtcgagccctgccctgccctgccttgccctgccctgccctgccctgcccggcacaccccaatcatttagcttagcgcccgcccacatatgccaacccccgagcgaggctcaatgttcgattctcgatgcgtgacgatgcttcgttacgctcggtcctccgcagccaggggttatataagtacgctcggtccgcttggtccaacatggtttgctcctaccaccatccgatccgtctactagctaccatcgtattctacccttctactgcactactgacagtcaaacaccttataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcgataacccagacatgaacaagctccagttccatttgatgctggatgaatttttctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttataactttgagttcagtttgggaactatttaaacctgctttaaatgaggaaaaaggaattcatcctatgttcttctattccataccgttccactcaaaacttgaacaaggttgttgttccaagttgtttttttaccgaattctcctctccctctaccctttgatgtcttttctgatcaactagtaccactttctcggtttcttcctcttggttgcacaaccactctctgtgtaccgactttgaaaagacttgtatcattgcatgcgcttgaacgctatcaattatcattcatagtgggtttatagttttgtccttgccatccacaatgtaccatagaccatcgctgccatggctgcgcgttgcgcgctgccacccagaccaccttgttccaggtccgatacaaaatcatgttagtcaattgagcacgggccacccaagggtgacggtcacccctaaggctccgggagtagtaaggcactataccagtctcttttacctccatgcgattcctaccatggtgcatcgcatctagaatctaggattctaggaatctagaaatctaggaatctaggatctggttaaccccaaaccatgtttattagacaggctcaatgggcaggtagggttagcgtgcaaggcagcgccgaggaaccggtcaggagatggggagagcaagtgcaaggtgagatgcccgaaatttgaaatttgaaatgtaaaaccccccgattggctatcgggctcggcgctggcagggctggaattggttaggcacgacataagtctgtaggcactaggctaggtaggcactagccgtcctcctatcctcgttgctgggcgctgggcgctggaagcttcagagcttcagattagtatgatagatactgcctcagtactgtgcgcacccatgcgctcccatgcatcccagctcctagctggtagcagcttgggagcttgggagcttggcatgttggcaagccacatcatatgcatctcatcatgcaatcatatcttttccccttgacaacttacattggttgtac

SEQ ID NO: 73 - DNA, 3119 bp, synthetic construct
  source 1..3119: mol_type = other DNA
cgtcacggagagtcaccttgcgcgagtagccacctacgtcatatgcgtcatatgcgtcatatgatatttatgtttttcaatacgcttcgagcgttcgactgtttgtccgttcgactgttcgactgctcgactgttcgactgttcgactgctcgactgttcgactgttcgactgttcgaccagcttagctaactaacccattctgattcaagagcttgagcgaacttgagcgagcttgaacccttgaaccaattgcccggttgttgcctgcccctggcccccgtccccagtgccccgtccccgtcccccgatcgactggcccccatgcggcttacgtctttgcagcgggctgccggcagccggcagtggacaaccgacagcttggcgcgcttgatgcatgatgcatgatgcttgacaggcagacagctggagtgctggagtgcactgaagctcggaagctgattcgctgagtcgctgagtcgctgagttgctgaatcgctgaacctaagagtgctacgatcttctactgaataattgaatttgaagattctatataaaccgactgttcatagatctctcctttgtcccggactacctctacatctacatccttctactgcactactgacagtcaaacaccttataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggaacgactttagatgataccgcctaccgttataggacatcagtaccgggcgatgcagaagcaattgaagcccttgatggatcattcaccacagacacagtttttcgcgtgacagctactggtgacggatttactctccgtgaagtgcctgtagatccgccattgacgaaagtatttccagatgatgagagtgatgacgagagcgatgatggcgaagatggtgatcctgactcgcgcactttcgtcgcatacggtgatgatggtgacctggccggatttgttgttgtgtcctactcggggtggaaccgtagattgaccgtcgaagatatcgaagtcgcgccagaacatcgtggtcatggcgtgggacgagcactgatgggtctagctacggaattcgctcgagagcgtggggcgggtcacttatggcttgaagttaccaatgtaaatgctcctgctattcacgcttatcgccgaatgggtttcaccttgtgtggcctcgacacggcattgtatgacggtactgcttctgacggggagcaagcgctatacatgtctatgccctgcccctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttataactttgagttcagtttgggaactatttaaacctgcttcctcctttattgaaccactcttgtggctaattaatccaatgccttgccgccctcaaatacacattgtttttaaatgttaattgaatattcaaatgcaagttgtatactctacatttttgcattcccaaccttttttttatcaacttttgtcttagctgctttacaatagttccatagcgctgaattccctttgggatacatgcacgtcagaaggattgttggcactcggaaactggtcgcagctaaatgccgaattgcctgttgctctggtgcaagtaaacaaacacaaggtcaagaaactggaataacagaatgtttccttcttgctttgattgctttgatcaggcacatctacagtatcttatggggcaagaaggctagagtaggccaaaacaagataccatcatggatggggtagtacttccatccctggtcaaccgatgagactacgaagaagacatactgttgaataattacgatgactaacgttttttggacaaggggaaagaacgtagcgtagctgcttcagaatgaatctaacggtagatcaggatctgaatgccctggttgaaacgcttgagaacctgagagcctaagatccatg

SEQ ID NO: 74 - DNA, 3906 bp, synthetic construct
  source 1..3906: mol_type = other DNA
ccgcatcagcacctcagcctctcagtctctttcggtcaggtcccaaattttcagcatcatgccgagagcgaaactagcgaagcgtaaagtgaggcgttgagacgttgagactttgggatctcatggcatcgtggatctcatgcatcatcatgcatcaggatcaggaatctcgttcgatctcatgaccatgtaagcggggcatagcggtccagtaacgtcatacagcctgcgattcccgccggcttaactcgccgctatgctgcgctcagccgctactgagacactgagggcgctgaggcgctgaggcggcatgagatctatggcatagcggcctaacatcgagaccttgaagcgttgaaatttgaaacgtgaagcgcgtgaagctagaagctggaaacttgactgccctcgcggcgacaaagccagaaataccgtggggtaaactgagcaattcaagcctcctaacacggagaccagctccacctgctcagcgctcagcgcgctcagcgccagcagggttagtgcacgacccagttttgtcggctacatcatgcataccattcattcgtctgcatcgatcatagatcatagatcatctacttgcatgcatgatactatgatgtacatcatgcatcatggtatcatggtattatggtacatgtgcacctcaacgcgcaatcacagtcgagcggagcttgaatcagaccagcgaaggaagctgatcgaaacggtgcccccaattactgagaccgatctgcttgaccctgcgcatcctattgctgcgcgctgacctcatgccccctcatgtcgggctggatggataccacacccgctggggaagtcttcgtcattggcagttgattgaaaaccttcgggggatgcccatgcgcaccgtgctatataaatggctgttgttctcatctcatcaaggtcccagaacaacttcttcatctgttgttactaccaccactcccaaccagcctgaagaaccaacatcatccttctactgcactactgacagtcaaacaccttataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatccatcatgcatcatgcatcatgcatcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggaacgactttagatgataccgcctaccgttataggacatcagtaccgggcgatgcagaagcaattgaagcccttgatggatcattcaccacagacacagtttttcgcgtgacagctactggtgacggatttactctccgtgaagtgcctgtagatccgccattgacgaaagtatttccagatgatgagagtgatgacgagagcgatgatggcgaagatggtgatcctgactcgcgcactttcgtcgcatacggtgatgatggtgacctggccggatttgttgttgtgtcctactcggggtggaaccgtagattgaccgtcgaagatatcgaagtcgcgccagaacatcgtggtcatggcgtgggacgagcactgatgggtctagctacggaattcgctcgagagcgtggggcgggtcacttatggcttgaagttaccaatgtaaatgctcctgctattcacgcttatcgccgaatgggtttcaccttgtgtggcctcgacacggcattgtatgacggtactgcttctgacggggagcaagcgctatacatgtctatgccctgcccctaatggtgttcaccactgttgcttcccctttgttggagcgtccgtcgttctgatggcgctctgagcccaaaatatcccttcttcgcccgagagcaagcctcggagtgtgcttgcaatctacttttcctactaaagttatcttcttgtggcttctgaatttgaatagaacgtgttcagacttgttagctaaatttgttgatgcgagatacttgacagaatcccgttgatgaactcatctgtgtgaccaccgcctggtgatggaatctcgcgcggaagccgtggtgttactgactctcaagcaattcagtagataaatttgtataacatcattgcaaggggtgctagcaatcaattgtccaataccaaacggcggcaagtgcaaaggggcgatagagagtttctgcgaggaagggaaggaatccgtatgcctgaaaacttttgtacaagtgtctttgtaagggcgaggtctgctctgacatctctctgagatctgagatctgatacctgcaggataacttcgtataatgtatgctatacgaagttataactttgagttcagtttgggaactatttaaacctgcttactatgatgcatgatgtactctacctgtgggtttaccgttttatcttaccttgtcaatcattaatcattgatcatccctgcaatttatttggaacgataccacctaccgtaatctcattggtgcaccctccccactaattttcttttccccactggtttacatcctactctgcaaacaaaccgctgatgtttagttcctcccggcagtcttattaattgttgtcgaagctctctttattagtaatacattttcctgatgcaccttgatcctagcttcaagcttcaagcttcaagatcttcatttgcatcccatagatcctgctgctaagcattccggaacgtaagagtaaattgtattttatggtcatgtttgagttagtcgaatttggtgtgaaccgagctcggcattctcaacgactcagcaactcagcacactcagcatactcagcaactcagtacaggctcagtacttagcttcaagtgcctcagcaaggaacccgagtcagtttggtcattggacctaagccacgggttaattccaagaaatgtagtaaatagggcaatgtcactggcaacttgtcaagtggagcacattcttcccctgataagctcctcgaatatccatcctgcatcagcacatcagtgcatcagcgcatcggcttaacgtgaccacccttacgataaacagctagaccatttcgaccatcttgatcatttcgaccattcggacatggagtcgaccatcgaagtcgtgagtcattaagccctgttgcctccggcgtcgggagcacgataccagaactgtagtgaggcgatgcaaatcaacaggtcataaacaactccactcgtcttctgccacttggtctgccttgtgtccttgtgtcaatcgggaataatgtggtaaggacaatattaggggctttgcacaattcaaagaatgcaatgccgttcggcacgatgtgctaactacaaggtacaacgtacacaacgtacaagtagc

SEQ ID NO: 75 - DNA, 4629 bp, synthetic construct
  source 1..4629: mol_type = other DNA
tggcatgtaggcctgtaggggctatgcctatatgctcatgcactcatgtcgcgatgttgcgatatcgcgatggaccaaccaagcaagcgtgcagcgtgggcgcttgggccaagggtgctaagctaagtgtaagcactgaagtgctgaagtgctgaagtgctgaacgtggaacgtcaggatgacacatcctctcccttcggcttcgtccaagaaaggttggtcagccatctgagatctgagatctgaaatgaatataatgatgaattgagtcgatgagatctgactatcttgggagattgcaccaatgcgccacggtgaccagatggtcttggtcttagctggtatagtggcacctgatctgccttgacagatctcatgttcatacgttcatgcattcaatcttatcgtctagccgtctgcgatctgcccaactagacgacgacaaatgtaattgatccttgtaaccctgttgctggtcgcagcagcgcgatacgatgccgtcacagtatcggcatctctgtctcgacgacagtgagtatagtcaactatgctatctagtagccaaagtaagtctacattccgggctttgcaggggttagctatttcattagacaatacttacaaccttcgaagggcttccccagaattcaattctatattgattcaatatccacccatcggacccgctctccgtaccccctgttcgcttcgccactacaaccatgtcaagacctgagaatcgtagcaaattgccccactacggcccgagtgctgtcggctggttaaagtagaatgacttgggtagccagatcgagacgcctgacattgatacgcagacagcgcagtcagtttcaaactgacaactgacgcgtactggattgacaggactccttggccggtgccattcgaatcgcttgtagacctcttgccagcatcttgccagagaagcttgaaggctggatcgctcagcctgagctcacacttagtagctccactagtcactcaggtgctcagcctctcctcagcgcctcaagctcaagcctcagggcctcagctgcatcgagctctggtgccagtgccagtgccagtgccagtgccagtgccagtgccagtgccagtgaagagagtggtatgagccaagctatacccaaaagcaaatgatggttattgccagcttccatgcacccctggggcgaaggacaacgaacaacagacagagtttgaattattcactggcctagtactaccaatttaaatttggtggttcagcgcaggaacggcgcgagtgagcgtaagggcaaactccgcatggggcgtgacgtcagtgtccctctggctgaggcactccagcaggctctggtcaacgcatcaacgtgtgtgtgtgtctaccaagcacattgtgcgctcgctgtccgtgcgccgaaatcgcaccagtcagaacgtctataaaacccctcttgcttggagggttaccgggccgtcttttcccaacgctccggctcactcactactctacacaacaaatgaccgacacaattgacgttgctgctttccaagcacacaaggctttccaaggcgaccaggagtcgtaccgtaagggctcgaaccgtcctggtttgcttgatggtgagcgtcgtcgctcatcggtttctgatgccaagatcaagctcaactcgggtcactacattcccgtcgtcgcgcttggtgtgtacaaggcacctaacgattcgactaccgaggatgccgtcaagtgggcttttgaggctggataccgtcatgttgattcgggtatgttacggttctctcccttgacctcttgatctctttgatctggtagctgaccattcatgaattgcaatctccagctgctcgatacgccaatgaggaagctgttggccgtgcattgaatgactttgtcaagactaccggtgtcccccgcgaggaaatctttgttacgtctaagctttgggacgcagaccacgaacgtgctgacgctgcgattgatgaatccttgcaaaagctcggtttgacgtacatggacttgtacctgttgcattcgcccggtgctgaccgtgacactcggttgaaggcttggaaagctatggaagatgctgtgtacgctggtaagatcaagtccattggtgtttccaactttgatgtagaagagttggatgacttgttggccaactgccgtattaagcctgctgtcaatcaaattgaatcgcatccattcttccaacatgaagatttgcgcaaagctaccattgaacgtggtattcacgtacaagcatattcgcctatggcacaaggtgctgcattggaacgtcctgagattgttgctattgccaagaagcacggtaagagtactgcacaaattttgctcaagtggggtatcaacattggtaacattgtgttacctaagagtttgactcgatcgcgtattgaagccaatgcagacttgtttgactttgaattggatcaagatgatatggatgtactgaacaatttggatgaaggtatgaagatgggcaaattgggtcctgctggtactagtgctcctggatcgcgtcgtacatctagtaactcgggtggcttgtctggtggtaaattacaaatgagttccaagtaacacgaagggtttcccatctgtgtgacctgatccctttttacgtttttttgttttgttttttcttttgttcatgatcaacttgggagtgattcctcttggtgatgtcgaaaattgaactgttataccaatattctgttgcctttgtttcctgtttgtagtttactactttttacagtagtcgttgttcctgccttgcttgcgtttgtcagggtttccctttagcttagtaccaatatctagccttaattttgcttattctttctcaccgcccatgattgatttcaatcatgattaatgatttatgcatgaaaactcaaaatactcaattgtcttgtacaaggcaatcacaagctggtcagccattctcagcgcctataagagctttcagccaggccggccccaaaacgatctaccctgtggacctggctaggctaaactttggaaagtcatgcaattatgaacgtttgaagctcaagcgctcctaccctctcctggagcagtgcttgaagactgagtccgtctcagataacttcgtataatgtatgctatacgaagttatcctgcaggagctcagaccttgcatgttagccttgaccttgcctctatgaccactaattcgcacaagattgcacgttaccattgtcatcttgccatgtatccaatgtttcctgtttgccatagtacgtagtagatacatcgatcatcgatgcagatgacgcagatggcattggttcgctttgccctatgcatgcatcatgcatcatgcatcatgcgtcatgcgtcactatccatattaatcattgccctctgtactaggccatgacacctgcagacccttccccagggatgcttcttgtaatagccaatatactacacaactttaccgattacgatcgagatcgatctcactatagtttgtcgaacttcgatgcacatcgagggtgcggaaaaaagctgacggaagcggaagtgggtgaattggttaatttagttagattggcagggttgttcccaagattcaacgcaagctgagaaagaaaacagaaacacgaggcataaggccagtcagaaaatgcattacgaggggagttgaagtgactgtgggccagtgcctgggtcgtcatgtgcaaattttttcttcggttagactgccgcagcatgcagcaactctgccggccctatataactagtgcttcatcttcttgtcattcttgtctctttaccaacacaccggtactctttcaccatgggtaaggaaaagacacacgtatcgcgaccccgactcaactccaacatggacgcagacctgtacggatacaaatgggctcgagataatgtcggacagtcgggtgcgacgatctacagactctatggaaaaccggacgcccccgagctctttcttaaacacggcaagggtagcgttgcaaacgacgtaaccgacgaaatggtccgtctgaattggctaactgagttcatgccactccccaccatcaagcacttcatccgtacacctgacgatgcgtggctgttgactacggccatccccggtaagaccgcttttcaggtcctcgaggaataccctgactcgggcgagaacatcgtggatgccttggctgtgtttttgcgccgccttcactctattccggtctgcaactgccctttcaacagcgaccgtgtttttaggcttgcccaggcacaatcacgcatgaacaatggtctagtcgacgctagtgatttcgatgacgagcgcaacggctggccggtggaacaagtgtggaaagagatgcataagctcctgcccttttccccagactctgtcgtgacgcatggtgatttctcgcttgacaacctcatctttgacgagggcaagctcattggctgcattgacgtcggccgggttggcatcgccgatcgctaccaagatcttgcgatcctctggaattgtctgggcgagttctcccctagcctccagaagcgtctgttccagaaatatgggatcg

SEQ ID NO: 76 - DNA, 1054 bp, Moniliella pollinis
  source 1..1054: mol_type = genomic DNA
atgtcctacaacaagaacatccctctcaacgacggtaactccatccctgcccttggctacggtacctggcaagcagaacctggtcaggtcggtgaaggtgtcaagctcgctgtcaaagctggctaccgtcacttggacttggccaaagtttaccagaaccaaaccgagattggccaagctctcaaggaactgtttgatgagggtgttgtcaagcgtgaggaccttttcatcacttccaagctttggaacaaccgccacgctcctgagcacgttgagcctgcgctcgacgagactcttaaggagctgggtctatcctatctggacctgtacttgattcacgtacgttttggccccttttgtgaattactatgaacatcagaagactaacttgattgtcgtgttgcagtggcctgttgctttcaagttcactactcccgatgaattgctccctgctgaccctaccaacaaggatcttgcctacgttgacgattcggtaaaattgtccgacacctggaaggcggtcgtcgccctgaagaagacgggtaagaccaagtcggttggtgtttcgaacttcagcactcgtctggtcgacttggttgaggaagcttcgggcgaacgtcctgcggtaaaccagatcgaagctcaccccttgttgcaacaagacgagttggttgctcaccataagagcaagaacattgtcatcactgcttacagtcccttgggcaacaatgtcgctggtaaaccacctctgactgagaaccccggtattgtggatgctgctaagcgtttgaaccatactcctgctgctgtgctcattgcttggggtattcaacgcgggtacagcgtcttggtcaagtcagttacaccctcacggatcaagagtaactttgaacagatcactctgtctgatgaggaattccaacgggttaccaacctcatcaaggagtacggtgagagccgtaacaacgttcctttcaattacaagccttcgtggtccattgacgtctttggtacccaggacgaggctaaggctacccacaagattaacgcttaa

SEQ ID NO: 77 - DNA, 984 bp, Moniliella pollinis
  source 1..984: mol_type = other DNA
atgtcctacaacaagaacatccctctcaacgacggtaactccatccctgcccttggctacggtacctggcaagcagaacctggtcaggtcggtgaaggtgtcaagctcgctgtcaaagctggctaccgtcacttggacttggccaaagtttaccagaaccaaaccgagattggccaagctctcaaggaactgtttgatgagggtgttgtcaagcgtgaggaccttttcatcacttccaagctttggaacaaccgccacgctcctgagcacgttgagcctgcgctcgacgagactcttaaggagctgggtctatcctatctggacctgtacttgattcactggcctgttgctttcaagttcactactcccgatgaattgctccctgctgaccctaccaacaaggatcttgcctacgttgacgattcggtaaaattgtccgacacctggaaggcggtcgtcgccctgaagaagacgggtaagaccaagtcggttggtgtttcgaacttcagcactcgtctggtcgacttggttgaggaagcttcgggcgaacgtcctgcggtaaaccagatcgaagctcaccccttgttgcaacaagacgagttggttgctcaccataagagcaagaacattgtcatcactgcttacagtcccttgggcaacaatgtcgctggtaaaccacctctgactgagaaccccggtattgtggatgctgctaagcgtttgaaccatactcctgctgctgtgctcattgcttggggtattcaacgcgggtacagcgtcttggtcaagtcagttacaccctcacggatcaagagtaactttgaacagatcactctgtctgatgaggaattccaacgggttaccaacctcatcaaggagtacggtgagagccgtaacaacgttcctttcaattacaagccttcgtggtccattgacgtctttggtacccaggacgaggctaaggctacccacaagattaacgct

SEQ ID NO: 78 - protein, 328 aa, Moniliella pollinis
MSYNKNIPLNDGNSIPALGYGTWQAEPGQVGEGVKLAVKAGYRHLDLAKVYQNQTEIGQALKELFDEGVVKREDLFITSKLWNNRHAPEHVEPALDETLKELGLSYLDLYLIHWPVAFKFTTPDELLPADPTNKDLAYVDDSVKLSDTWKAVVALKKTGKTKSVGVSNFSTRLVDLVEEASGERPAVNQIEAHPLLQQDELVAHHKSKNIVITAYSPLGNNVAGKPPLTENPGIVDAAKRLNHTPAAVLIAWGIQRGYSVLVKSVTPSRIKSNFEQITLSDEEFQRVTNLIKEYGESRNNVPFNYKPSWSIDVFGTQDEAKATHKINA

SEQ ID NO: 79 - DNA, 1093 bp, Moniliella pollinis
  source 1..1093: mol_type = genomic DNA
atgtcttacaaacagtacatccccctgaacgacggtaacaaaatccctgcccttgggtttggtacttggcaagctgaacctggtcaagtgggtgcaagtgtcaagaacgctgtcaaggctgggtaccgtcatttggatttggccaaagtgtaccaaaaccaatcggaaattggagtagcacttaaggaactgtttgatcaaggtattgtgaaacgggaagatttgtttattacgtccaaagtatggaataaccgtcatgctcctgaacatgttgagcctgcattggacgaaacattgaaagaacttggattgtcctacttggatttgtacttgattcatgtacgttttgatgtttccttcctatttttttttcttcttgccttacgaaagtcacgaaaggaaggagggggaactgactttcgatcacgattgtacatagtggcccgttgcgttcaagtttactacgcctcaggaactattccctactgagccggataacaaggaattggtcgcgattgatgattcaatcaagttggtagacacttggaaggcagttgtagcactcaaaaaaacgggtaagaccaaatccgttggtgtgtcgaacttcactacggatttggtagacttggttgaaaaagcgtcgggggaacgaccggcggtcaatcagattgaagcacacccattgttacaacaggatgaattggttgctcatcacaagagtaaaaacattgtgattactgcgtacagtcctttgggaaacaatgtgagtgggaaaccacctctgactcaaaaccctgggattgaagcaactgcaaaacggttaaatcatactcctgctgcggtcttgcttgcgtgggggattcaacgtggatacagtgtcttggtcaagagtgttacaccttctcgcattgagagcaattatgatcagattaccctttctcctgaagaattccagaaggttactgatctgatcaaggaatatggcgaaagtcgcaacaatattccgttgaattataagccttcatggccgatcagtgtgtttggtacggcggatgaagctaaggctactcataagattaacaccaacctttga

SEQ ID NO: 80 - DNA, 990 bp, Moniliella pollinis
  source 1..990: mol_type = other DNA
atgtcttacaaacagtacatccccctgaacgacggtaacaaaatccctgcccttgggtttggtacttggcaagctgaacctggtcaagtgggtgcaagtgtcaagaacgctgtcaaggctgggtaccgtcatttggatttggccaaagtgtaccaaaaccaatcggaaattggagtagcacttaaggaactgtttgatcaaggtattgtgaaacgggaagatttgtttattacgtccaaagtatggaataaccgtcatgctcctgaacatgttgagcctgcattggacgaaacattgaaagaacttggattgtcctacttggatttgtacttgattcattggcccgttgcgttcaagtttactacgcctcaggaactattccctactgagccggataacaaggaattggtcgcgattgatgattcaatcaagttggtagacacttggaaggcagttgtagcactcaaaaaaacgggtaagaccaaatccgttggtgtgtcgaacttcactacggatttggtagacttggttgaaaaagcgtcgggggaacgaccggcggtcaatcagattgaagcacacccattgttacaacaggatgaattggttgctcatcacaagagtaaaaacattgtgattactgcgtacagtcctttgggaaacaatgtgagtgggaaaccacctctgactcaaaaccctgggattgaagcaactgcaaaacggttaaatcatactcctgctgcggtcttgcttgcgtgggggattcaacgtggatacagtgtcttggtcaagagtgttacaccttctcgcattgagagcaattatgatcagattaccctttctcctgaagaattccagaaggttactgatctgatcaaggaatatggcgaaagtcgcaacaatattccgttgaattataagccttcatggccgatcagtgtgtttggtacggcggatgaagctaaggctactcataagattaacaccaacctt

SEQ ID NO: 81 - protein, 330 aa, Moniliella pollinis
MSYKQYIPLNDGNKIPALGFGTWQAEPGQVGASVKNAVKAGYRHLDLAKVYQNQSEIGVALKELFDQGIVKREDLFITSKVWNNRHAPEHVEPALDETLKELGLSYLDLYLIHWPVAFKFTTPQELFPTEPDNKELVAIDDSIKLVDTWKAVVALKKTGKTKSVGVSNFTTDLVDLVEKASGERPAVNQIEAHPLLQQDELVAHHKSKNIVITAYSPLGNNVSGKPPLTQNPGIEATAKRLNHTPAAVLLAWGIQRGYSVLVKSVTPSRIESNYDQITLSPEEFQKVTDLIKEYGESRNNIPLNYKPSWPISVFGTADEAKATHKINTNL